SEQUENCE LISTING (85 sequences)

SEQ ID NO: 1 - DNA, 1102 bp, Homo sapiens
gcgccgcaaggcactgagcaggcgaaagagcgcgctcggacctccttcccggcggcagctaccgagagtgcggagcgaccagcgtgcgctcggaggaaccagagaaactcagcaccccgcgggactgtccgtcgcaaaatccaacatgaaaatcctcgtggccttggcagtcttttttcttgtctccactcagctgtttgcagaagaaataggagccaatgatgatctgaattactggtccgactggtacgacagcgaccagatcaaggaggaactgccggagccctttgagcatcttctgcagagaatcgcccggagacccaagcctcagcagttctttggattaatgggcaaacgggatgctgattcctcaattgaaaaacaagtggccctgttaaaggctctttatggacatggccagatctctcacaaaagacataaaacagattcctttgttggactaatgggcaaaagagctttaaattctgtggcttatgaaaggagtgcaatgcagaattatgaaagaagacgttaataaactacctaacattatttattcagcttcatttgtgtcaatgggcaatgacaggtaaattaagacatgcactatgaggaataattatttatttaataacaattgtttggggttgaaaattcaaaaagtgtttatttttcatattgtgccaatatgtattgtaaacatgtgttttaattccaatatgatgactcccttaaaatagaaataagtggttatttctcaacaaagcacagtgttaaatgaaattgtaaaacctgtcaatgatacagtccctaaagaaaaaaaatcattgctttgaagcagttgtgtcagctactgcggaaaaggaaggaaactcctgacagtcttgtgcttttcctatttgttttcatggtgaaaatgtactgagattttggtattacactgtatttgtatctctgaagcatgtttcatgttttgtgactatatagagatgtttttaaaagtttcaatgtgattctaatgtcttcatttcattgtatgatgtgttgtgatagctaacattttaaataaaagaaaaaatatcttgaaaaaaaaaaaaaaaaa

SEQ ID NO: 2 - DNA, 2768 bp, Homo sapiens
ccgaggcgctccctgggatcacatggtacctgctccagtgccgcgtgcggcccgggaaccctgggctgctggcgcctgcgcagagccctctgtcccagggaaaggctcgggcaaaaggcggctgagattggcagagtgaaatattactgccgagggaacgtagcagggcacacgtctcgcctctttgcgactcggtgccccgtttctccccatcacctacttacttcctggttgcaacctctcttcctctgggacttttgcaccgggagctccagattcgctaccccgcagcgctgcggagccggcaggcagaggcaccccgtacactgcagagacccgaccctccttgctaccttctagccagaactactgcaggctgattccccctacacactctctctgctcttcccatgcaaagcagaactccgttgcctcaacgtccaacccttctgcagggctgcagtccggccaccccaagaccttgctgcagggtgcttcggatcctgatcgtgagtcgcggggtccactccccgcccttagccagtgcccagggggcaacagcggcgatcgcaacctctagtttgagtcaaggtccagtttgaatgaccgctctcagctggtgaagacatgacgaccctggactccaacaacaacacaggtggcgtcatcacctacattggctccagtggctcctccccaagccgcaccagccctgaatccctctatagtgacaactccaatggcagcttccagtccctgacccaaggctgtcccacctacttcccaccatcccccactggctccctcacccaagacccggctcgctcctttgggagcattccacccagcctgagtgatgacggctccccttcttcctcatcttcctcgtcgtcatcctcctcctccttctataatgggagcccccctgggagtctacaagtggccatggaggacagcagccgagtgtcccccagcaagagcaccagcaacatcaccaagctgaatggcatggtgttactgtgtaaagtgtgtggggacgttgcctcgggcttccactacggtgtgcacgcctgcgagggctgcaagggctttttccgtcggagcatccagcagaacatccagtacaaaaggtgtctgaagaatgagaattgctccatcgtccgcatcaatcgcaaccgctgccagcaatgtcgcttcaagaagtgtctctctgtgggcatgtctcgagacgctgtgcgttttgggcgcatccccaaacgagagaagcagcggatgcttgctgagatgcagagtgccatgaacctggccaacaaccagttgagcagccagtgcccgctggagacttcacccacccagcaccccaccccaggccccatgggcccctcgccaccccctgctccggtcccctcacccctggtgggcttctcccagtttccacaacagctgacgcctcccagatccccaagccctgagcccacagtggaggatgtgatatcccaggtggcccgggcccatcgagagatcttcacctacgcccatgacaagctgggcagctcacctggcaacttcaatgccaaccatgcatcaggtagccctccagccaccaccccacatcgctgggaaaatcagggctgcccacctgcccccaatgacaacaacaccttggctgcccagcgtcataacgaggccctaaatggtctgcgccaggctccctcctcctaccctcccacctggcctcctggccctgcacaccacagctgccaccagtccaacagcaacgggcaccgtctatgccccacccacgtgtatgcagccccagaaggcaaggcacctgccaacagtccccggcagggcaactcaaagaatgttctgctggcatgtcctatgaacatgtacccgcatggacgcagtgggcgaacggtgcaggagatctgggaggatttctccatgagcttcacgcccgctgtgcgggaggtggtagagtttgccaaacacatcccgggcttccgtgacctttctcagcatgaccaagtcaccctgcttaaggctggcacctttgaggtgctgatggtgcgctttgcttcgttgttcaacgtgaaggaccagacagtgatgttcctaagccgcaccacctacagcctgcaggagcttggtgccatgggcatgggagacctgctcagtgccatgttcgacttcagcgagaagctcaactccctggcgcttaccgaggaggagctgggcctcttcaccgcggtggtgcttgtctctgcagaccgctcgggcatggagaattccgcttcggtggagcagctccaggagacgctgctgcgggctcttcgggctctggtgctgaagaaccggcccttggagacttcccgcttcaccaagctgctgctcaagctgccggacctgcggaccctgaacaacatgcattccgagaagctgctgtccttccgggtggacgcccagtgacccgcccggccggccttctgccgctgcccccttgtacagaatcgaactctgcacttctctctcctttacgagacgaaaaggaaaagcaaaccagaatcttatttatattgttataaaatattccaagatgagcctctggccccctgagccttcttgtaaatacctgcctccctcccccatcaccgaacttcccctcctcccctatttaaaccactctgtctcccccacaaccctcccctggccctctgatttgttctgttcctgtctcaaatccaatagttcacagctgagctggg

SEQ ID NO: 3 - DNA, 5473 bp, Homo sapiens
ggcaggacgaggtggcaccaaattcccttcggccaatgacgagccggagtttacagaagcctcattagcatttccccagaggcaggggcaggggcagaggccgggtggtgtggtgtcggtgtcggcagcatccccggcgccctgctgcggtcgccgcgagcctcggcctctgtctcctccccctcccgcccttacctccacgcgggaccgcccgcgccagtcaactcctcgcactttgcccctgcttggcagcggataaaagggggctgaggaaataccggacacggtcacccgttgccagctctagcctttaaattcccggctcggggacctccacgcaccgcggctagcgccgacaaccagctagcgtgcaaggcgccgcggctcagcgcgtaccggcgggcttcgaaaccgcagtcctccggcgaccccgaactccgctccggagcctcagccccctggaaagtgatcccggcatccgagagccaagatgccggcccacttgctgcaggacgatatctctagctcctataccaccaccaccaccattacagcgcctccctccagggtcctgcagaatggaggagataagttggagacgatgcccctctacttggaagacgacattcgccctgatataaaagatgatatatatgaccccacctacaaggataaggaaggcccaagccccaaggttgaatatgtctggagaaacatcatccttatgtctctgctacacttgggagccctgtatgggatcactttgattcctacctgcaagttctacacctggctttggggggtattctactattttgtcagtgccctgggcataacagcaggagctcatcgtctgtggagccaccgctcttacaaagctcggctgcccctacggctctttctgatcattgccaacacaatggcattccagaatgatgtctatgaatgggctcgtgaccaccgtgcccaccacaagttttcagaaacacatgctgatcctcataattcccgacgtggctttttcttctctcacgtgggttggctgcttgtgcgcaaacacccagctgtcaaagagaaggggagtacgctagacttgtctgacctagaagctgagaaactggtgatgttccagaggaggtactacaaacctggcttgctgatgatgtgcttcatcctgcccacgcttgtgccctggtatttctggggtgaaacttttcaaaacagtgtgttcgttgccactttcttgcgatatgctgtggtgcttaatgccacctggctggtgaacagtgctgcccacctcttcggatatcgtccttatgacaagaacattagcccccgggagaatatcctggtttcacttggagctgtgggtgagggcttccacaactaccaccactcctttccctatgactactctgccagtgagtaccgctggcacatcaacttcaccacattcttcattgattgcatggccgccctcggtctggcctatgaccggaagaaagtctccaaggccgccatcttggccaggattaaaagaaccggagatggaaactacaagagtggctgagtttggggtccctcaggttcctttttcaaaaaccagccaggcagaggttttaatgtctgtttattaactactgaataatgctaccaggatgctaaagatgatgatgttaacccattccagtacagtattcttttaaaattcaaaagtattgaaagccaacaactctgcctttatgatgctaagctgatattatttcttctcttatcctctctctcttctaggcccattgtcctccttttcactttattgctatcgccctcctttcccttattgcctcccaggcaagcagctggtcagtctttgctcagtgtccagcttccaaagcctagacaacctttctgtagcctaaaacgaatggtctttgctccagataactctctttccttgagctgttgtgagctttgaagtaggtggcttgagctagagataaaacagaatcttctgggtagtcccctgttgattatcttcagcccaggcttttgctagatggaatggaaaagcaacttcatttgacacaaagcttctaaagcaggtaaattgtcgggggagagagttagcatgtatgaatgtaaggatgagggaagcgaagcaagaggaacctctcgccatgatcagacatacagctgcctacctaatgaggacttcaagccccaccacatagcatgcttcctttctctcctggctcggggtaaaaagtggctgcggtgtttggcaatgctaattcaatgccgcaacatatagttgaggccgaggataaagaaaagacattttaagtttgtagtaaaagtggtctctgctggggaagggttttcttttctttttttctttaataacaaggagatttcttagttcatatatcaagaagtcttgaagttgggtgtttccagaattggtaaaaacagcagctcatagaattttgagtattccatgagctgctcattacagttctttcctctttctgctctgccatcttcaggatattggttcttcccctcatagtaataagatggctgtggcatttccaaacatccaaaaaaagggaaggatttaaggaggtgaagtcgggtcaaaaataaaatatatatacatatatacattgcttagaacgttaaactattagagtatttcccttccaaagagggatgtttggaaaaaactctgaaggagaggaggaattagttgggatgccaatttcctctccactgctggacatgagatggagaggctgagggacaggatctataggcagcttctaagagcgaacttcacataggaagggatctgagaacacgttgccaggggcttgagaaggttactgagtgagttattgggagtcttaataaaataaactagatattaggtccattcattaattagttccagtttctccttgaaatgagtaaaaactagaaggcttctctccacagtgttgtgccccttcactcatttttttttgaggagaagggggtctctgttaacatctagcctaaagtatacaactgcctggggggcagggttaggaatctcttcactaccctgattcttgattcctggctctaccctgtctgtcccttttctttgaccagatctttctcttccctgaacgttttcttctttccctggacaggcagcctcctttgtgtgtattcagaggcagtgatgacttgctgtccaggcagctccctcctgcacacagaatgctcagggtcactgaaccactgcttctcttttgaaagtagagctagctgccactttcacgtggcctccgcagtgtctccacctacacccctgtgctcccctgccacactgatggctcaagacaaggctggcaaaccctcccagaaacatctctggcccagaaagcctctctctccctccctctctcatgaggcacagccaagccaagcgctcatgttgagccagtgggccagccacagagcaaaagagggtttattttcagtcccctctctctgggtcagaaccagagggcatgctgaatgccccctgcttacttggtgagggtgccccgcctgagtcagtgctctcagctggcagtgcaatgcttgtagaagtaggaggaaacagttctcactgggaagaagcaagggcaagaacccaagtgcctcacctcgaaaggaggccctgttccctggagtcagggtgaactgcaaagctttggctgagacctgggatttgagataccacaaaccctgctgaacacagtgtctgttcagcaaactaaccagcattccctacagcctagggcagacaatagtatagaagtctggaaaaaaacaaaaacagaatttgagaaccttggaccactcctgtccctgtagctcagtcatcaaagcagaagtctggctttgctctattaagattggaaatgtacactaccaaacactcagtccactgttgagccccagtgctggaagggaggaaggcctttcttctgtgttaattgcgtagaggctacaggggttagcctggactaaaggcatccttgtcttttgagctattcacctcagtagaaaaggatctaagggaagatcactgtagtttagttctgttgacctgtgcacctaccccttggaaatgtctgctggtatttctaattccacaggtcatcagatgcctgcttgataatatataaacaataaaaacaactttcacttcttcctattgtaatcgtgtgccatggatctgatctgtaccatgaccctacataaggctggatggcacctcaggctgagggccccaatgtatgtgtggctgtgggtgtgggtgggagtgtgtctgctgagtaaggaacacgattttcaagattctaaagctcaattcaagtgacacattaatgataaactcagatctgatcaagagtccggatttctaacagtccttgctttggggggtgtgctgacaacttagctcaggtgccttacatcttttctaatcacagtgttgcatatgagcctgccctcactccctctgcagaatccctttgcacctgagaccctactgaagtggctggtagaaaaaggggcctgagtggaggattatcagtatcacgatttgcaggattcccttctgggcttcattctggaaacttttgttagggctgcttttcttaagtgcccacatttgatggagggtggaaataatttgaatgtatttgatttataagttttttttttttttttgggttaaaagatggttgtagcatttaaaatggaaaattttctccttggtttgctagtatcttgggtgtattctctgtaagtgtagctcaaataggtcatcatgaaaggttaaaaaagcgaggtggccatgttatgctggtggttaaggccagggcctctccaaccactgtgccactgacttgctgtgtgaccctgggcaagtcacttaactataaggtgcctcagttttccttctgttaaaatggggataataatactgacctacctcaaagggcagttttgaggcatgactaatgctttttagaaagcattttgggatccttcagcacaggaattctcaagacctgagtattttttataataggaatgtccaccatgaacttgatacgtccgtgtgtcccagatgctgtcattagtctatatggttctccaagaaactgaatgaatccattggagaagcggtggataactagccagacaaaatttgagaatacataaacaacgcattgccacggaaacatacagaggatgccttttctgtgattgggtgggattttttccctttttatgtgggatatagtagttacttgtgacaagaataattttggaataatttctattaatatcaactctgaagctaattgtactaatctgagattgtgtttgttcataataaaagtgaagtgaatctgattgcaaaaaa

SEQ ID NO: 4 - DNA, 639 bp, Homo sapiens
gagagaaggagagcctgcagacagaggcctccagcttggtctgtctccccacctctaccagcatctgctgagctatgagccaaaccagggatttacagggaggaaaagctttcggactgctgaaggcccagcaggaagagaggctggatgagatcaacaagcaattcctagacgatcccaaatatagcagtgatgaggatctgccctccaaactggaaggcttcaaagagaaatacatggagtttgaccttaatggaaatggcgatattgatatcatgtccctgaaacgaatgctggagaaacttggagtccccaagactcacctagagctaaagaaattaattggagaggtgtccagtggctccggggagacgttcagctaccctgactttctcaggatgatgctgggcaagagatctgccatcctaaaaatgatcctgatgtatgaggaaaaagcgagagaaaaggaaaagccaacaggccccccagccaagaaagctatctctgagttgccctgatttgaagggaaaagggatgatgggattgaaggggcttctaatgacccagatatggaaacagaagacaaaattgtaagccagagtcaacaaattaaataaattaccccctcctccagatcaa

SEQ ID NO: 5 - DNA, 1190 bp, Homo sapiens
cagatccatcaggtccgagctgtgttgactaccacttttcccttcgtctcaattatgtcttggaaaaaggctttgcggatccccggaggccttcgggcagcaactgtgaccttgatgctgtcgatgctgagcaccccagtggctgagggcagagactctcccgaggatttcgtgtaccagtttaagggcatgtgctacttcaccaacgggacagagcgcgtgcgtcttgtgagcagaagcatctataaccgagaagagatcgtgcgcttcgacagcgacgtgggggagttccgggcggtgacgctgctggggctgcctgccgccgagtactggaacagccagaaggacatcctggagaggaaacgggcggcggtggacagggtgtgcagacacaactaccagttggagctccgcacgaccttgcagcggcgagtggagcccacagtgaccatctccccatccaggacagaggccctcaaccaccacaacctgctggtctgctcggtgacagatttctatccagcccagatcaaagtccggtggtttcggaatgaccaggaggagacagctggcgttgtgtccaccccccttattaggaatggtgactggaccttccagatcctggtgatgctggaaatgactccccagcgtggagacgtctacacctgccacgtggagcaccccagcctccagagccccatcaccgtggagtggcgggctcaatctgaatctgcccagagcaagatgctgagtggcattggaggcttcgtgctggggctgatcttcctcgggctgggccttatcatccatcacaggagtcagaaagggctcctgcactgactcctgagactattttaactgggattggttatcacttttctgtaacgcctgcttgtccctgcccagaattcccagctgtctgtgtcagcctgtccccctgagatcagagtcctacagtggctgtcacgcagccaccaggtcatctcctttcatccccaccttgaggcggatggctgtgaccctacttcctgcactgacccacagcctctgcctgtgcacggccagctgcatctactcaggccccaaggggtttctgtttctattctctcctcagactgctcaagagaagcacatgaaaaccattacctgactttagagcttttttacataattaaacatgatcctgagtt

SEQ ID NO: 6 - DNA, 2823 bp, Homo sapiens
tgtgtcatttcctttcttcatgtaccagatgctgaaatactatgagataaagattttaggtttcaattgtaaagagagagaagtggataaatcagtgctgctttctttaggacgaaagaagtatggagcagtgggatcactttcacaatcaacaggaggacactgatagctgctccgaatctgtgaaatttgatgctcgctcaatgacagctttgcttcctccgaatcctaaaaacagcccttcccttcaagagaaactgaagtccttcaaagctgcactgattgccctttacctcctcgtgtttgcagttctcatccctctcattggaatagtggcagctcaactcctgaagtgggaaacgaagaattgctcagttagttcaactaatgcaaatgatataactcaaagtctcacgggaaaaggaaatgacagcgaagaggaaatgagatttcaagaagtctttatggaacacatgagcaacatggagaagagaatccagcatattttagacatggaagccaacctcatggacacagagcatttccaaaatttcagcatgacaactgatcaaagatttaatgacattcttctgcagctaagtaccttgttttcctcagtccagggacatgggaatgcaatagatgaaatctccaagtccttaataagtttgaataccacattgcttgatttgcagctcaacatagaaaatctgaatggcaaaatccaagagaataccttcaaacaacaagaggaaatcagtaaattagaggagcgtgtttacaatgtatcagcagaaattatggctatgaaagaagaacaagtgcatttggaacaggaaataaaaggagaagtgaaagtactgaataacatcactaatgatctcagactgaaagattgggaacattctcagaccttgagaaatatcactttaattcaaggtcctcctggacccccgggtgaaaaaggagatcgaggtcccactggagaaagtggtccacgaggatttccaggtccaataggtcctccgggtcttaaaggtgatcggggagcaattggctttcctggaagtcgaggactcccaggatatgccggaaggccaggaaattctggaccaaaaggccagaaaggggaaaaggggagtggaaacacattaagaccagtacaactcactgatcatattagggcagggccctcttaagatcaggtgggttgggcgggacatcctctgctaccatctcattaaaaggcccttcacctctggacaagtcatctgcacaactgacttccaagatccttttgtgactcctccaaatgactttggttcccgtgttgtacctgacttccacatggccttctctcctggtccctggtgctgtttgggcctctgctcccatgctcatacctcttcttactccaattactccaccatcacctctctcccctatcacccccagcctggacacctctcatgcacggactggagggctgctccaaccagtcctcagttctctgccacccattgacctagagtcttgaacccaatttaatttattgggttctaggagaactgctgtgttctcaccctaacttggaagagtgatgtttcagtcaagcaaagcgattcctaccatacaatataacacttgtgtgaggctctgtcctaaatatctcaattaccaatatgtggtttggtagtatttctcgccatgctttgctcatgcgcaatgagactacaactagggtgtaaattttaagtatcccatctaaaactcatacaatgataggaaaaatccatttgtttttcatttgatttttactgaggaatcagctcaatcttcaatgaatactggtctctttccaaagcatttttgatcaaagtaaagactgagtcaagggctttttttttttctttttcttgttttaagagacagagccttgttctattgcacaggctggactacacgcattcacctagagtctagaacacaatttaatttattgggttctaggagaactgtcatgagtattgataatatgagagttctttatattcaaacattattctcaaccagagatagggatgtcatagaagaaaatccattcattcaatcattaattcacatgtccattatgtacctccatgagctggacataacagctaataagagataattgtctctggttttacagagctaattgtccctaagagatgtagacaaatgaacaagcaattacaatacatctaagctatactgggggaggaacagggctggataggtatgcagaggagataaaaaaattttaattccttagaatattttttaaaaattgattcttattttaccttctcatcttcttattttccaaattacagcatatatatatatttttttaagttttgaagtgtagtcgagcttgggcaatttatccaacccatttaaaccaaaaataaaacttttcatgtattacctggtcatttcaaacaaaaatattttgatcatgaaaaagaataccaatattcttttgttctaaaaatctcttatgggattacatgttatatttttggtttctctctactgatcaacagactacattttcacaactcttctttcctttacgttttaacacacagacccaagattcatactattaagattctagtagaactctagatggtatgcctctgtgtatctcagcatttttattcccactcttgtataatgaacatgttaacacctacctcacagggttgttgtgaggatcaagtaagatattgtgtgtgtgaagatgctctgtgaaatcataaagtcctttaaagatgt

SEQ ID NO: 7 - DNA, 3682 bp, Homo sapiens
tgtgtcatttcctttcttcatgtaccagatgctgaaatactatgagataaagattttaggtttcaattgtaaagagagagaagtggataaatcagtgctgctttctttaggacgaaagaagtatggagcagtgggatcactttcacaatcaacaggaggacactgatagctgctccgaatctgtgaaatttgatgctcgctcaatgacagctttgcttcctccgaatcctaaaaacagcccttcccttcaagagaaactgaagtccttcaaagctgcactgattgccctttacctcctcgtgtttgcagttctcatccctctcattggaatagtggcagctcaactcctgaagtgggaaacgaagaattgctcagttagttcaactaatgcaaatgatataactcaaagtctcacgggaaaaggaaatgacagcgaagaggaaatgagatttcaagaagtctttatggaacacatgagcaacatggagaagagaatccagcatattttagacatggaagccaacctcatggacacagagcatttccaaaatttcagcatgacaactgatcaaagatttaatgacattcttctgcagctaagtaccttgttttcctcagtccagggacatgggaatgcaatagatgaaatctccaagtccttaataagtttgaataccacattgcttgatttgcagctcaacatagaaaatctgaatggcaaaatccaagagaataccttcaaacaacaagaggaaatcagtaaattagaggagcgtgtttacaatgtatcagcagaaattatggctatgaaagaagaacaagtgcatttggaacaggaaataaaaggagaagtgaaagtactgaataacatcactaatgatctcagactgaaagattgggaacattctcagaccttgagaaatatcactttaattcaaggtcctcctggacccccgggtgaaaaaggagatcgaggtcccactggagaaagtggtccacgaggatttccaggtccaataggtcctccgggtcttaaaggtgatcggggagcaattggctttcctggaagtcgaggactcccaggatatgccggaaggccaggaaattctggaccaaaaggccagaaaggggaaaaggggagtggaaacacattaactccatttacgaaagttcgactggtcggtgggagcggccctcacgaggggagggtggagatactccacagcggccagtggggtacaatttgtgacgatcgctgggaagtgcgcgttggacaggtcgtctgtaggagcttgggatacccaggtgttcaagccgtgcacaaggcagctcactttggacaaggtactggtccaatatggctgaatgaagtgttttgttttgggagagaatcatctattgaagaatgtaaaattcggcaatgggggacaagagcctgttcacattctgaagatgctggagtcacttgcactttataatgcatcatattttcattcacaactatgaaatcgctgctcaaaaatgattttattaccttgttcctgtaaaatccatttaatcaatatttaagagattaagaatattgcccaaataatattttagattacaggattaatatattgaacaccttcatgcttactattttatgtctatatttaaatcattttaacttctataggtttttaaatggaattttctaatataatgacttatatgctgaattgaacattttgaagtttatagcttccagattacaaaggccaagggtaatagaaatgcataccagtaattggctccaattcataatatgttcaccaggagattacaattttttgctcttcttgtctttgtaatctatttagttgattttaattactttctgaataacggaagggatcagaagatatcttttgtgcctagattgcaaaatctccaatccacacatattgttttaaaataagaatgttatccaactattaagatatctcaatgtgcaataacttgtgtattagatatcaatgttaatgatatgtcttggccactatggaccagggagcttatttttcttgtcatgtactgacaactgtttaattgaatcatgaagtaaattgaaagcaggacatatgagaaaactgaccatcagtatatttgtccagataattggtggatcaaaaatgccacttaacaggaagtttagtttgttatgcactttaaatggaataattagcttgttacaattctaggacatggtgtttaaaatttaaatctgattaatccattttaacaaacaatgcaaacatcttcagtgcagaaggaagagtggtttcaactgtttggagtcttttatgaagtcagtcaacatgtacaaccaaagggcgggggggggggtggggggtgcgtctttagtcctaaagggacaataactctgagcatgccccaaaaaagtagtttagcaaccttttgttggtagtcaacccatccccagggccatagtgtagagtgtgaaaagctaccctgaaacccagtaattctaccctgaaagtgactgcctgcagaaagaccagcagttgatattaaagcgcaaatgaattcaacctcagccctgaaaataacagaattctgaagtttcctatgactaattcacaaaaaaagtaattgtaaactagtactattatggaattactctactgttctttctttaatagtggcaaatgaaagcataagcttaagcattttttcatattctgaagtctcaccacacataataaccaagtggtagactcacagccgtccaacttaaaaaggcaaaaccttaccttggaattggaattactgtaaacagcctactgaaaatgcatttttatcatgtaacattcttctacttgtttaacattgctgattttctctggcagcataattttgtggttaagagaatgaattctgaatgtacactttctgtctcaaaccctggctgtaatttcagctagttaataattctttgtgttcagttccactatctaggtattttcttcaaaaggtaaatacaatggtttctgaaagaatcatttgcattatcagcctgtttgggatgtctgagatcagtgcctctgggttgttaatactgtattgctgtatggtatatgtatgctgatttactacttatgcgtaagtggtatgcatgggatgtctgaaatcagtgcctatgggttgtcaatagtattaactattagtgttaactgttagtattaactattagtattattaacactaataatagtactattactattactatttttattttaaaataaaatttacctttaaaataataatagtactattgctagtactagtactattgctattactagtactattactagtactagtactatgacactgttaatagtactattaacaacccataggcacttgggatgtctgagatcagtgcctatgggttgttaatactatattgctgtatggtatatgcatgctgatttaccacttatgcatagatatatctttaataagtaatctaaaaatcctttttgtatttgagagaatctactaagttcagtccagtcaagaaaagaacctaatagcaccaatacaaattgaggacttaatttactttggaatgttgaattgcatttgttccattaaaaaaaacagaaatttg

SEQ ID NO: 8 - DNA, 2436 bp, Homo sapiens
attcatttcccccagtgaccttgacaagtcagaagcttgaaagcagggaaatccggatgtctcggttatgaagtggagcagtgagtgtgagcctcaacatagttccagaactctccatccggactagttattgagcatctgcctctcatatcaccagtggccatctgaggtgtttccctggctctgaaggggtaggcacgatggccaggtgcttcagcctggtgttgcttctcacttccatctggaccacgaggctcctggtccaaggctctttgcgtgcagaagagctttccatccaggtgtcatgcagaattatggggatcacccttgtgagcaaaaaggcgaaccagcagctgaatttcacagaagctaaggaggcctgtaggctgctgggactaagtttggccggcaaggaccaagttgaaacagccttgaaagctagctttgaaacttgcagctatggctgggttggagatggattcgtggtcatctctaggattagcccaaaccccaagtgtgggaaaaatggggtgggtgtcctgatttggaaggttccagtgagccgacagtttgcagcctattgttacaactcatctgatacttggactaactcgtgcattccagaaattatcaccaccaaagatcccatattcaacactcaaactgcaacacaaacaacagaatttattgtcagtgacagtacctactcggtggcatccccttactctacaatacctgcccctactactactcctcctgctccagcttccacttctattccacggagaaaaaaattgatttgtgtcacagaagtttttatggaaactagcaccatgtctacagaaactgaaccatttgttgaaaataaagcagcattcaagaatgaagctgctgggtttggaggtgtccccacggctctgctagtgcttgctctcctcttctttggtgctgcagctggtcttggattttgctatgtcaaaaggtatgtgaaggccttcccttttacaaacaagaatcagcagaaggaaatgatcgaaaccaaagtagtaaaggaggagaaggccaatgatagcaaccctaatgaggaatcaaagaaaactgataaaaacccagaagagtccaagagtccaagcaaaactaccgtgcgatgcctggaagctgaagtttagatgagacagaaatgaggagacacacctgaggctggtttctttcatgctccttaccctgccccagctggggaaatcaaaagggccaaagaaccaaagaagaaagtccacccttggttcctaactggaatcagctcaggactgccattggactatggagtgcaccaaagagaatgcccttctccttattgtaaccctgtctggatcctatcctcctacctccaaagcttcccacggcctttctagcctggctatgtcctaataatatcccactgggagaaaggagttttgcaaagtgcaaggacctaaaacatctcatcagtatccagtggtaaaaaggcctcctggctgtctgaggctaggtgggttgaaagccaaggagtcactgagaccaaggctttctctactgattccgcagctcagaccctttcttcagctctgaaagagaaacacgtatcccacctgacatgtccttctgagcccggtaagagcaaaagaatggcagaaaagtttagcccctgaaagccatggagattctcataacttgagacctaatctctgtaaagctaaaataaagaaatagaacaaggctgaggatacgacagtacactgtcagcagggactgtaaacacagacagggtcaaagtgttttctctgaacacattgagttggaatcactgtttagaacacacacacttactttttctggtctctaccactgctgatattttctctaggaaatatacttttacaagtaacaaaaataaaaactcttataaatttctatttttatctgagttacagaaatgattactaaggaagattactcagtaatttgtttaaaaagtaataaaattcaacaaacatttgctgaatagctactatatgtcaagtgctgtgcaaggtattacactctgtaattgaatattattcctcaaaaaattgcacatagtagaacgctatctgggaagctatttttttcagttttgatatttctagcttatctacttccaaactaatttttatttttgctgagactaatcttattcattttctctaatatggcaaccattataaccttaatttattattaacatacctaagaagtacattgttacctctatataccaaagcacattttaaaagtgccattaacaaatgtatcactagccctcctttttccaacaagaagggactgagagatgcagaaatatttgtgacaaaaaattaaagcatttagaaaacttaaaaaaaaaaaaaaaaaaaaaaa

SEQ ID NO: 9 - DNA, 1360 bp, Mus musculus
gccttccccagtcaggcaagaagcagcaaggtacaagaatacacagctccaggctccaagggtcctgtgcgctcaggaagttggtgcggacaatgttcatcttgcttgtgttagtaactggagtttctgctttcaccactccaacagtggtgcacacaggaaaggtttctgaatcccccattacatcggagaagcccacagtccatggagacaactgtcagtttcgtggcagagagttcaaatctgaattgaggctggaaggtgaacctgtggttctgaggtgccccttggcacctcactccgacatctccagcagttcccatagttttctgacctggagtaaattggactcttctcagctgatcccaagagatgagccaaggatgtgggtgaagggtaacatactctggattctgccagcagtgcagcaagactctggtacctacatttgcacattcagaaacgcatcccactgtgagcaaatgtctgtggaactcaaggtctttaagaatactgaagcatctctgcctcatgtctcctacttgcaaatctcagctctctccaccaccgggttactagtgtgccctgacctgaaagaattcatctccagcaacgctgatggaaagatacagtggtataagggcgccatactcttggataaaggcaataaggaatttctgagtgcaggagaccccacacgcctattgatatccaacacgtccatggacgatgcaggctattacagatgtgttatgacatttacctacaatggccaggaatacaacatcactaggaatattgaactccgggtcaaaggaacaaccacggaacccatccctgtgatcatttctcccctggagacaataccagcatcattggggtcaagactgatagtcccgtgcaaagtgtttctgggaactggtacatcttccaacaccattgtgtggtggttggctaacagcacgtttatctcggctgcttacccaagaggccgtgtgaccgaggggctacaccaccagtactcagagaatgatgaaaactatgtggaagtgtcgctgatttttgatccagtcacaagggaggatctgcatacagattttaaatgtgttgcctcgaatccacggagttctcagtcactccataccacagtcaaagaagtctcttccacgttctcctggagcattgcgctggcacctctgtctctgatcatcttggttgtgggggcaatatggatgcgcagacggtgtaaacgcagggctggaaagacatatggactgaccaagctacggactgacaaccaggacttcccttccagcccaaactaaataaaggaaatgaaataaaaaaaaaaaaaaaaaaa

SEQ ID NO: 10 - DNA, 2601 bp, Homo sapiens
ccgcccttgtaggctgtccacctcaaacgggccggacaggatatataagagagaatgcaccgtgcactacacacgcgactcccacaaggttgcagccggagccgcccagctcaccgagagcctagttccggccagggtcgccccggcaaccacgagcccagccaatcagcgccccggactgcaccagagccatggtcggcagaagagcactgatcgtactggctcactcagagaggacgtccttcaactatgccatgaaggaggctgctgcagcggctttgaagaagaaaggatgggaggtggtggagtcggacctctatgccatgaacttcaatcccatcatttccagaaaggacatcacaggtaaactgaaggaccctgcgaactttcagtatcctgccgagtctgttctggcttataaagaaggccatctgagcccagatattgtggctgaacaaaagaagctggaagccgcagaccttgtgatattccagttccccctgcagtggtttggagtccctgccattctgaaaggctggtttgagcgagtgttcataggagagtttgcttacacttacgctgccatgtatgacaaaggacccttccggagtaagaaggcagtgctttccatcaccactggtggcagtggctccatgtactctctgcaagggatccacggggacatgaatgtcattctctggccaattcagagtggcattctgcatttctgtggcttccaagtcttagaacctcaactgacatatagcattgggcacactccagcagacgcccgaattcaaatcctggaaggatggaagaaacgcctggagaatatttgggatgagacaccactgtattttgctccaagcagcctctttgacctaaacttccaggcaggattcttaatgaaaaaagaggtacaggatgaggagaaaaacaagaaatttggcctttctgtgggccatcacttgggcaagtccatcccaactgacaaccagatcaaagctagaaaatgagattccttagcctggatttccttctaacatgttatcaaatctgggtatctttccaggcttccctgacttgctttagtttttaagatttgtgtttttctttttccacaaggaataaatgagagggaatcgactgtattcgtgcatttttggatcatttttaactgattcttatgattactatcatggcatataaccaaaatccgactgggctcaagaggccacttagggaaagatgtagaaagatgctagaaaaatgttctttaaaggcatctacacaatttaattcctctttttagggctaaagttttagggtacagtttggctaggtatcattcaactctccaatgttctattaatcacctctctgtagtttatggcagaagggaattgctcagagaaggaaaagactgaatctacctgccctaagggacttaacttgtttggtagttagccatctaatgcttgtttatgatatttcttgctttcaattacaaagcagttactaatatgcctagcacaagtaccactcttggtcagcttttgttgtttatatacagtacacagataccttgaaaggaagagctaataaatctcttctttgctgcagtcatctactttttttttaattaaaaaaaatttttttttgaagcagtcttgctctgttacccaggctggagtgcagtggtgtgatctcggctcactgcaacctctgcctcccaggttccagcaattctcctgcctcagcctccctagtagctgggatgacaggcgcctgccatcatgcctgactaatttttgtatttttagtagagacggcgtttcaccatgttggccaggctggtctcaaactcctgacctcaggtgatccgcctacctcagcctcccaaagtgctgggattacaggcgtgatccaccacacctggcccttgcaatcttctactttaaggtttgcagagataaaccaataaatccacaccgtacatctgcaatatgaattcaagaaaggaaatagtaccttcaatacttaaaaatagtcttccacaaaaaatactttatttctgatctatacaaattttcagaaggttattttctttatcattgctaaactgatgacttactatgggatggggtccagtcccatgaccttggggtacaattgtaaacctagagttttatcaactttggtgaacagttttggcataatagtcaatttctacttctggaagtcatctcattccactgttggtattatataattcaaggagaatatgataaaacactgccctcttgtggtgcattgaaagaagagatgagaaatgatgaaaaggttgcctgaaaaatgggagacagcctcttacttgccaagaaaatgaagggattggaccgagctggaaaacctcctttaccagatgctgactggcactggtggtttttgctctcgacagtatccacaatagctgacggctgggtgtttcagtttgaaaatattttgttgccttcatcttcactgcaattttgtgtaaatttctcaaagatctgaattaaataaataaaattcatttctacagacccacaaaaaaaaaaa

SEQ ID NO: 11 - DNA, 1853 bp, Homo sapiens
gccaaagggaagtgctgcgaggtttacaaccagctgcagtggttcgatgggaaggatctttctccaagtggttcctcttgaggggagcatttctgctggctccaggactttggccatctataaagcttggcaatgagaaataagaaaattctcaaggaggacgagctcttgagtgagacccaacaagctgcttttcaccaaattgcaatggagcctttcgaaatcaatgttccaaagcccaagaggagaaatggggtgaacttctccctagctgtggtggtcatctacctgatcctgctcaccgctggcgctgggctgctggtggtccaagttctgaatctgcaggcgcggctccgggtcctggagatgtatttcctcaatgacactctggcggctgaggacagcccgtccttctccttgctgcagtcagcacaccctggagaacacctggctcagggtgcatcgaggctgcaagtcctgcaggcccaactcacctgggtccgcgtcagccatgagcacttgctgcagcgggtagacaacttcactcagaacccagggatgttcagaatcaaaggtgaacaaggcgccccaggtcttcaaggtcacaagggggccatgggcatgcctggtgcccctggcccgccgggaccacctgctgagaagggagccaagggggctatgggacgagatggagcaacaggcccctcgggaccccaaggcccaccgggagtcaagggagaggcgggcctccaaggaccccagggtgctccagggaagcaaggagccactggcaccccaggaccccaaggagagaagggcagcaaaggcgatgggggtctcattggcccaaaaggggaaactggaactaagggagagaaaggagacctgggtctcccaggaagcaaaggggacaggggcatgaaaggagatgcaggggtcatggggcctcctggagcccaggggagtaaaggtgacttcgggaggccaggcccaccaggtttggctggttttcctggagctaaaggagatcaaggacaacctggactgcagggtgttccgggccctcctggtgcagtgggacacccaggtgccaagggtgagcctggcagtgctggctcccctgggcgagcaggacttccagggagccccgggagtccaggagccacaggcctgaaaggaagcaaaggggacacaggacttcaaggacagcaaggaagaaaaggagaatcaggagttccaggccctgcaggtgtgaagggagaacaggggagcccagggctggcaggtcccaagggagcccctggacaagctggccagaagggagaccagggagtgaaaggatcttctggggagcaaggagtaaagggagaaaaaggtgaaagaggtgaaaactcagtgtccgtcaggattgtcggcagtagtaaccgaggccgggctgaagtttactacagtggtacctgggggacaatttgcgatgacgagtggcaaaattctgatgccattgtcttctgccgcatgctgggttactccaaaggaagggccctgtacaaagtgggagctggcactgggcagatctggctggataatgttcagtgtcggggcacggagagtaccctgtggagctgcaccaagaatagctggggccatcatgactgcagccacgaggaggacgcaggcgtggagtgcagcgtctgacccggaaaccctttcacttctctgctcccgaggtgtcctcgggctcatatgtgggaaggcagaggatctctgaggagttccctggggacaactgagcagcctctggagaggggccattaataaagctcaacatcattggcaaaaaaaaaaaaaaaaaa

SEQ ID NO: 12 - DNA, 3740 bp, Homo sapiens
cgccgtctgccctccgcagcgctcgcccctttctctgggaggacaacctgctgacccgaagccagggtctgccagacaaccacgaccaactagtcccagataaccttgaggcctgggcactggctgggccccgagggctcttcccaaagcgtaccctggtcatctggaagaggatcggagctggcctggtggtgacagtggccttgcttcctaggatggatggcagatggcaatgttcctgctgggcctggttcctgctggttctggcagttgtagctggggacacagtgtcaaccgggtccacggacaacagcccaacatccaatagcctggaggggggcaccgacgccacggccttctggtggggggagtggaccaagtggacggcgtgttcccgcagttgcgggggtggggtgacatcccaggagcggcactgcctgcagcagaggaggaagtccgtcccgggccccgggaacaggacctgcacgggcacgtccaagcggtaccagctctgcagagtgcaggagtgtccgccggacgggaggagcttccgcgaggagcagtgcgtctccttcaactcccacgtgtacaacgggcggacgcaccagtggaagcctctgtacccggatgactatgtccacatctccagcaaaccgtgtgacctgcactgtaccaccgtggacggccagcggcagctcatggtccccgcccgcgacggcacatcctgcaagctcactgacctgcgaggggtttgcgtgtctggaaaatgtgagcccatcggctgtgacggggtgcttttctccacccacacactggacaagtgtggcatctgccagggggacggtagcagctgcacccacgtgacgggcaactatcgcaaggggaatgcccaccttggttactctctggtgacccacatcccggctggtgcccgagacatccagattgtagagaggaagaagtccgctgacgtgctagctcttgcagatgaagctggctactacttcttcaacggcaactacaaggtggacagccccaagaacttcaacatcgctggcacggtggtcaagtaccggcggcccatggatgtctatgagaccggaatcgagtacatcgtggcacaggggcccaccaaccagggcctgaatgtcatggtgtggaaccagaacggcaaaagcccctccatcaccttcgagtacacgctgctgcagccgccacacgagagccgcccccagcccatctactatggcttctccgagagcgctgagagccagggcctggacggggccgggctgatgggcttcgtcccgcacaacggctccctctacggccaggcctcctcagagcggctgggcctggacaaccggctgttcggccacccgggcctggacatggagctgggccccagccagggccaggagaccaacgaggtgtgcgagcaggccggcggcggggcctgcgaggggccccccaggggcaagggcttccgagaccgcaacgtcacggggactcctctcaccggggacaaggatgacgaagaggttgacacccacttcgcctcccaggagttcttctcggctaacgccatctctgaccagctgctgggcgcaggctctgacttgaaggacttcaccctcaatgagactgtgaacagcatctttgcacagggcgccccaaggagctccctggccgagagcttcttcgtggattatgaggagaacgagggggctggcccttacctgctcaacgggtcctacctggagctgagcagcgacagggttgccaacagctcctccgaggccccattccccaacgttagcaccagcctgctcacctcggccgggaacaggactcacaaggccaggaccaggcccaaggcgcgcaagcaaggcgtgagtcccgcggacatgtaccggtggaagctctcgtcccacgagccctgcagtgccacctgcaccacaggggtcatgtctgcgtacgccatgtgtgtccgctatgatggcgtcgaggtggatgacagctactgtgacgccctgacccgtcccgagcctgtccacgagttctgcgctgggagggagtgccagcccaggtgggagacgagcagctggagcgagtgttcgcgcacctgcggagagggctaccagttccgcgtcgtgcgctgctggaagatgctctcgcccggcttcgacagctccgtgtacagcgacctgtgcgaggcagccgaggccgtgcggcccgaggaacgcaagacctgccggaaccccgcctgcgggccccagtgggagatgtcggagtggtccgagtgcactgccaagtgtggggagcgcagtgtggtgaccagggacatccgctgctcggaggatgagaagctgtgtgaccccaacaccaggcctgtaggggagaagaactgcacgggcccgccctgtgaccggcagtggaccgtctccgactggggaccgtgcagtggaagctgcgggcaaggccgcaccatcaggcacgtgtactgcaagaccagcgacggacgggtggtacctgagtcccagtgccagatggagaccaagcctctggccatccacccctgtggggacaaaaactgtcccgcccactggctggcccaggactgggagcggtgcaacaccacctgcgggcgcggggtcaagaagcggctggtgctctgcatggagctggccaacgggaagccgcagacgcgcagtggccccgagtgcgggctcgccaagaagcctcccgaggagagcacgtgtttcgagaggccctgcttcaagtggtacaccagcccctggtcagagtgcaccaagacctgcggggtgggcgtgaggatgcgagacgtcaagtgctaccaggggaccgacatcgtccgtggttgcgatccgttggtgaagcccgttggcagacaggcctgtgatctgcagccctgccccacggagcccccagatgacagctgccaggaccagccaggcaccaactgtgccctggccatcaaagtgaacctctgcgggcactggtactacagcaaggcgtgctgccgctcctgcaggcccccccactcctaggcccggcagctgcagccccttccagatgaagaccaagcgcccctcctggggctgctgcagcttctggggcctccacagacccccctcctgcggggcacgctggcctaagagacgtggcactgagcctcggctgtcgagaggggacttcccacggcccatggacctttgtgctcctggggcagagcctccggcacccagtggcctcccccagacagagccacccctgccgtgggaacctgtccgtgttcctgcgtggatcctgtgtttgtggctcccactccccagccccccagcagcccccagccgaggggcccagggcccacagccagcggtggaggtgtcttgctccgggcccgtagcccacgccctctctgggtggcagggccttctgaaggaaacttgcaggcgagcccaacgtggtggggggccttcctccctcagaggccatggggtgagaggggctcaggcagccaaggaggcccaggcgtgctccctcttatggagcccctcccatggagctctcttcccgccgcactttctaccccgggcagaggcgcttgcccacgggacgtttggggatggacctcggcccccgcccctgcagtcagcgtcagtgctcatctacgttaataaagtggtcctatttatggcggcaaaaaaaaaaaaaaa

SEQ ID NO: 13 - DNA, 4007 bp, Homo sapiens
ctaaaaccttgcaagttcaggaagaaaccatctgcatccatattgaaaacctgacacaatgtatgcagcaggctcagtgtgagtgaactggaggcttctctacaacatgacccaaaggagcattgcaggtcctatttgcaacctgaagtttgtgactctcctggttgccttaagttcagaactcccattcctgggagctggagtacagcttcaagacaatgggtataatggattgctcattgcaattaatcctcaggtacctgagaatcagaacctcatctcaaacattaaggaaatgataactgaagcttcattttacctatttaatgctaccaagagaagagtatttttcagaaatataaagattttaatacctgccacatggaaagctaataataacagcaaaataaaacaagaatcatatgaaaaggcaaatgtcatagtgactgactggtatggggcacatggagatgatccatacaccctacaatacagagggtgtggaaaagagggaaaatacattcatttcacacctaatttcctactgaatgataacttaacagctggctacggatcacgaggccgagtgtttgtccatgaatgggcccacctccgttggggtgtgttcgatgagtataacaatgacaaacctttctacataaatgggcaaaatcaaattaaagtgacaaggtgttcatctgacatcacaggcatttttgtgtgtgaaaaaggtccttgcccccaagaaaactgtattattagtaagctttttaaagaaggatgcacctttatctacaatagcacccaaaatgcaactgcatcaataatgttcatgcaaagtttatcttctgtggttgaattttgtaatgcaagtacccacaaccaagaagcaccaaacctacagaaccagatgtgcagcctcagaagtgcatgggatgtaatcacagactctgctgactttcaccacagctttcccatgaatgggactgagcttccacctcctcccacattctcgcttgtacaggctggtgacaaagtggtctgtttagtgctggatgtgtccagcaagatggcagaggctgacagactccttcaactacaacaagccgcagaattttatttgatgcagattgttgaaattcataccttcgtgggcattgccagtttcgacagcaaaggagagatcagagcccagctacaccaaattaacagcaatgatgatcgaaagttgctggtttcatatctgcccaccactgtatcagctaaaacagacatcagcatttgttcagggcttaagaaaggatttgaggtggttgaaaaactgaatggaaaagcttatggctctgtgatgatattagtgaccagcggagatgataagcttcttggcaattgcttacccactgtgctcagcagtggttcaacaattcactccattgccctgggttcatctgcagccccaaatctggaggaattatcacgtcttacaggaggtttaaagttctttgttccagatatatcaaactccaatagcatgattgatgctttcagtagaatttcctctggaactggagacattttccagcaacatattcagcttgaaagtacaggtgaaaatgtcaaacctcaccatcaattgaaaaacacagtgactgtggataatactgtgggcaacgacactatgtttctagttacgtggcaggccagtggtcctcctgagattatattatttgatcctgatggacgaaaatactacacaaataattttatcaccaatctaacttttcggacagctagtctttggattccaggaacagctaagcctgggcactggacttacaccctgaacaatacccatcattctctgcaagccctgaaagtgacagtgacctctcgcgcctccaactcagctgtgcccccagccactgtggaagcctttgtggaaagagacagcctccattttcctcatcctgtgatgatttatgccaatgtgaaacagggattttatcccattcttaatgccactgtcactgccacagttgagccagagactggagatcctgttacgctgagactccttgatgatggagcaggtgctgatgttataaaaaatgatggaatttactcgaggtattttttctcctttgctgcaaatggtagatatagcttgaaagtgcatgtcaatcactctcccagcataagcaccccagcccactctattccagggagtcatgctatgtatgtaccaggttacacagcaaacggtaatattcagatgaatgctccaaggaaatcagtaggcagaaatgaggaggagcgaaagtggggctttagccgagtcagctcaggaggctccttttcagtgctgggagttccagctggcccccaccctgatgtgtttccaccatgcaaaattattgacctggaagctgtaaaagtagaagaggaattgaccctatcttggacagcacctggagaagactttgatcagggccaggctacaagctatgaaataagaatgagtaaaagtctacagaatatccaagatgactttaacaatgctattttagtaaatacatcaaagcgaaatcctcagcaagctggcatcagggagatatttacgttctcaccccaaatttccacgaatggacctgaacatcagccaaatggagaaacacatgaaagccacagaatttatgttgcaatacgagcaatggataggaactccttacagtctgctgtatctaacattgcccaggcgcctctgtttattccccccaattctgatcctgtacctgccagagattatcttatattgaaaggagttttaacagcaatgggtttgataggaatcatttgccttattatagttgtgacacatcatactttaagcaggaaaaagagagcagacaagaaagagaatggaacaaaattattataaataaatatccaaagtgtcttccttcttagatataagacccatggccttcgactacaaaaacatactaacaaagtcaaattaacatcaaaactgtattaaaatgcattgagtttttgtacaatacagataagatttttacatggtagatcaacaaattctttttgggggtagattagaaaacccttacactttggctatgaacaaataataaaaattattctttaaagtaatgtctttaaaggcaaagggaagggtaaagtcggaccagtgtcaaggaaagtttgttttattgaggtggaaaaatagccccaagcagagaaaaggagggtaggtctgcattataactgtctgtgtgaagcaatcatttagttactttgattaatttttcttttctccttatctgtgcagaacaggttgcttgtttacaactgaagatcatgctatattttatatatgaagcccctaatgcaaagctctttacctcttgctattttgttatatatattacagatgaaatctcactgctaatgctcagagatcttttttcactgtaagaggtaacctttaacaatatgggtattacctttgtctcttcataccggttttatgacaaaggtctattgaatttatttgtttgtaagtttctactcccatcaaagcagctttctaagttattgccttggttattatggatgatagttatagcccttataatgccttaactaaggaagaaaagatgttattctgagtttgttttaatacatatatgaacatatagttttattcaattaaaccaaagaagaggtcagcagggagatactaacctttggaaatgattagctggctctgttttttggttaaataagagtctttaatcctttctccatcaagagttacttaccaagggcaggggaagggggatatagaggtcacaaggaaataaaaatcatctttcatctttaattttactccttcctcttatttttttaaaagattatcgaacaataaaatcatttgcctttttaattaaaaaaaaaaaaaaaaaaaaaaaaaaaaaaaaaaaaaaaaa

SEQ ID NO: 14 - DNA, 5054 bp, Homo sapiens
actcagcctggagaccgaagcgcttcactgagcgctcgccgccgcccagcctctcctctcgcgcctcctagctcttcgcagagcaaccaggagccaggagtggtctagagcccgagggtgggaagggggagtctgtctggcttttctcctatcttgcttctttttcctcttcccttcccactcttgttcaagcgagtgtgtgagctatggagcgaagagcctggagtctgcagtgcactgctttcgtcctcttttgcgcttggtgtgcactgaacagtgcaaaagcgaaaaggcaatttgtcaatgaatgggcagcggagatccccgggggcccggaagcagcctcggccatcgccgaggagctgggctatgaccttttgggtcagattggttcacttgaaaatcactacttattcaaacataaaaaccaccccagaaggtctcgaaggagtgcctttcatatcactaagagattatctgatgatgatcgtgtgatatgggctgaacaacagtatgaaaaagaaagaagtaaacgttcagctctaagggactcagcactaaatctcttcaatgatcccatgtggaatcagcaatggtacttgcaagataccaggatgacggcagccctgcccaagctggaccttcatgtgatacctgtttggcaaaaaggcattacgggcaaaggagttgttatcaccgtactggatgatggtttggagtggaatcacacggacatttatgccaactatgatccagaggctagctatgattttaatgataatgaccatgatccatttccccgatatgatcccacaaacgagaacaaacacgggaccagatgtgcaggagaaattgccatgcaagcaaataatcacaaatgcggggttggagttgcatacaattccaaagttggaggcataagaatgctggatggcattgtgacggatgctattgaggccagttcaattggattcaatcctggacacgtggatatttacagtgcaagctggggccctaatgatgatgggaaaactgtggaggggcctggccggctagcccagaaggcttttgaatatggtgtcaaacaggggagacaggggaaggggtccatcttcgtctgggcttcgggaaacggggggcgtcagggagataattgtgactgtgatggctacacagacagcatctacaccatctccatcagcagtgcctcccagcaaggcctatccccctggtacgctgagaagtgctcctccacactggccacctcttacagcagcggagattacaccgaccagagaatcacgagcgctgacctgcacaatgactgcacggagacgcacacaggcacctcggcctctgcacctctggctgctggcatcttcgctctggccctggaagcaaacccaaatctcacctggcgagatatgcagcacctggttgtctggacctctgagtatgacccgctggccaataaccctggatggaaaaagaatggagcaggcttgatggtgaatagtcgatttggatttggcttgctaaatgccaaagctctggtggatttagctgaccccaggacctggaggagcgtgcctgagaagaaagagtgtgttgtaaaggacaatgactttgagcccagagccctgaaagctaatggagaagttatcattgaaattccaacaagagcttgtgaaggacaagaaaatgctatcaagtccctggagcatgtacaatttgaagcaacaattgaatattcccgaagaggagaccttcatgtcacacttacttctgctgctggaactagcactgtgctcttggctgaaagagaacgggatacatctcctaatggctttaagaactgggacttcatgtctgttcacacatggggagagaaccctataggtacttggactttgagaattacagacatgtctggaagaattcaaaatgaaggaagaattgtgaactggaagctgattttgcacgggacctcttctcagccagagcatatgaagcagcctcgtgtgtacacgtcctacaacactgttcagaatgacagaagaggggtggagaagatggtggatccaggggaggagcagcccacacaagagaaccctaaggagaacaccctggtgtccaaaagccccagcagcagcagcgtagggggccggagggatgagttggaggagggagccccttcccaggccatgctgcgactcctgcaaagtgctttcagtaaaaactcaccgccaaagcaatcaccaaagaagtccccaagtgcaaagctcaacatcccttatgaaaacttctacgaagccctggaaaagctgaacaaaccttcccagcttaaagactctgaagacagtctgtataatgactatgttgatgttttttataacactaaaccttacaagcacagagacgaccggctgcttcaagctctggtggacattctgaatgaggaaaattaaaataagtgtgtggtcccaagttggaaatattcatgcttcttccttaccctgcgattttgcctgtgtctgaagtggttgttttgtcatgaattcttatgcttataatatcctttgtggcaccttttctttttctccctaaactgtacatgtgaaggggatgagctcaagcaggaagttcaacttccagaattgatcataggtatttcaaaacacatctttcctgtctgcacaagtgaagtgttttgttctttctggagtcacagttgacaaaaagctcttacactacattagaacactgcattagagcccatttcaattctcaaaagaaaaggcaaaacctgggatatcaattaatttgaaaacataatctgcaaagaatgagaaggagtcagaaactgtttctgtagcttgttccctgtcttgtccatgtggttcttcaaattttgatgccaagaaagtatttggtaggcctaatgaaggagttcactgtaagactcattccctagatctttctattccaaagtgccactcattcctgtagtcaaaatctggtcatgttggtcaaaagctggattatttagatctagaaacagatcttgaaatctgaatgctctggtttgagcaattttcgaacattctttgcctggtgcactgtgtctgtggtgccagaggcgtccgtggatccagaggtggttatgactcgtgctgcatgcctggtctttcctctgtttctccttctgaaagttttctatacctgtctcctttctcagccacaaaataaatgttgggagaaatgatatataccactttcccagaaaaaaaaaaacttacacttgggacttggcaaattcctagtcacaatttttttcagcagtaacaggaaaccacttatcacatggagacctaatgtaataatagaaaaatactcataatagggagaaaccaagagaagttttgtttttgtttttttccaactgtgttcattagaacagcgtgttctaagtatttgaaactgaatgtttattccttgatactaaaagttcttctccaatcctatcactgatagtgtccaaattctcaccaaattgctcctaagcttcaaatcagaagcagaaactggcaggccatggaccttaattgtccctcaggtagattttgtttggtatgcagaatgtttttaaaatatgagtggttattgaaaatatgatgtttcacataaaacctcattctcggacccatctttgctcatggcaacagttagctggagctgagtagcagctgcctgattagatgactctcagtccccatggcaccctgctccatgttacctagagcaggcacttgattccttgctgggcagtatccaataggcatttgattttgcccactcctacactaagcgaatgtgtacaaagtgtaaatgcattaggaaaaacaaactacccgcatcttctgttaggcaggatctgtacaataataattatgagtttgcttatgtaatctcacctcacctggatgatcactaatactaattcatttattactaaccttctggcttccttctctcaatatgcttacaaagtctccagtcacctacaatgctggctttctcccactgagtttgctgtttgcaatttttccatgaagtttgaacttcataaggtaattcatggcattgaactggttcatgaaaagaacactagagtctgtcatttgctttggcttgaagtatggttggtaacacaaattttcacctgctcttctaccatttgaatttgtgtagagggtgtttgcagagcaatgcccgtaatgcttagagaatgttctcctaaaagacttgcggaatcactctgtccttggaagtttcatatattgtttgatatgaagtgttagatagaatttccaatattggagcatatcaaaaagtattaaaactaaaaaggaccagagaattcttagattggcccggaaaggccaataaagagttagaatgaaaactcattacttttccattcccaatctagtgctagatgtataaatctttcttttgattcttcctaacaaaatattttctgggttaaaaccccagccaactcattgggttgtagccaaaggttcactctcaagaagctttaatatttaaataaaatcatattgaatgtttccaacctggagtataatattcagatataaaacagttttgtcagtctttcttagtgcctgtgtggatttttgtgaaaatgtcaaagagaaaacttatatactatttcccttgaaattttaaactatattttctttacaggtatttataatataccaatgcttttatcaaacagaattttaaagagcataataaattatattaaagaaccaaaagttttcctgagaataagaaagtttcacccaataaaatatttttgaaaggcatgttcctctgtcaatgaaaaaaagtacatgtatgtgttgtgatattaaaagtgacatttgtctaatagcctaatacaacatgtagctgagtttaacatgtgtggtcttggtattcttaagggaacttccacattatacatttgatgtattgaccagaat

SEQ ID NO: 15 - DNA, 874 bp, Homo sapiens
aggaagcaaagaaggacctgggctttgggaagatctaaagacccaggaaggtctctgggtgggataaagccaagatgaaactccccttacttctggctcttctatttggggcagtttctgctcttcatctaaggtctgagacttccacctttgagacccctttgggtgctaagacgctgcctgaggatgaggagacaccagagcaggagatggaggagaccccttgcagggagctggaggaagaggaggagtggggctctggaagtgaagatgcctccaagaaagatggggctgttgagtctatctcagtgccagatatggtggacaaaaaccttacgtgtcctgaggaagaggacacagtaaaagtggtgggcatccctgggtgccagacctgccgctacctcctggtgagaagtcttcagacgtttagtcaagcttggtttacttgccggaggtgctacaggggcaacctggtttccatccacaacttcaatattaattatcgaatccagtgttctgtcagcgcgctcaaccagggtcaagtctggattggaggcaggatcacaggctcgggtcgctgcagacgctttcagtgggttgacggcagccgctggaactttgcgtactgggctgctcaccagccctggtcccgcggtggtcactgcgtggccctgtgtacccgaggaggccactggcgtcgagcccactgcctcagaagacttcctttcatctgttcctactgagctggtcccagccagcagttcagagctgccctctcctgggcagctgcctcccctcctctgcttgccatccctccctccacctccctgcaataaaatgggttttactgaaatggaaaaaaaaaaaaaaaaaa

SEQ ID NO: 16 - DNA, 4876 bp, Homo sapiens
ccgcagttcttgagttccacatgcagagcagatgcgacagctagaagtgagtagggcccagaccctggcccaggaagatccactaaaggaggccatccttccgccttcttctgcaggagtcaggatggaaaggcagatgtaaagtccctcatggcgaaatataacacggggggcaacccgacagaggatgtctcagtcaatagccgacccttcagagtcacagggccaaactcatcttcaggaatacaagcaagaaagaacttattcaacaaccaaggaaatgccagccctcctgcaggacccagcaatgtacctaagtttgggtccccaaagccacctgtggcagtcaaaccttcttctgaggaaaagcctgacaaggaacccaagcccccgtttctaaagcccactggagcaggccaaagattcggaacaccagccagcttgaccaccagagaccccgaggcgaaagtgggatttctgaaacctgtaggccccaagcccatcaacttgcccaaagaagattccaaacctacatttccctggcctcctggaaacaagccatctcttcacagtgtaaaccaagaccatgacttaaagccactaggcccgaaatctgggcctactcctccaacctcagaaaatgaacagaagcaagcgtttcccaaattgactggggttaaagggaaatttatgtcagcatcacaagatcttgaacccaagcccctcttccccaaacccgcctttggccagaagccgcccctaagtaccgagaactcccatgaagacgaaagccccatgaagaatgtgtcttcatcaaaagggtccccagctcccctgggagtcaggtccaaaagcggccctttaaaaccagcaagggaagactcagaaaataaagaccatgcaggggagatttcaagtttgccctttcctggagtggttttgaaacctgctgcgagcaggggaggcccaggtctctccaaaaatggtgaagaaaaaaaggaagataggaagatagatgctgctaagaacaccttccagagcaaaataaatcaggaagagttggcctcagggactcctcctgccaggttccctaaggccccttctaagctgacagtgggggggccatggggccaaagtcaggaaaaggaaaagggagacaagaattcagccaccccgaaacagaagccattgcctcccttgtttaccttgggtccacctccaccaaaacccaacagaccaccaaatgttgacctgacgaaattccacaaaacctcttctggaaacagtactagcaaaggccagacgtcttactcaacaacttccctgccaccacctccaccatcccatccggccagccaaccaccattgccagcatctcacccatcacaaccaccagtcccaagcctacctcccagaaacattaaacctccgtttgacctaaaaagccctgtcaatgaagacaatcaagatggtgtcacgcactctgatggtgctggaaatctagatgaggaacaagacagtgaaggagaaacatatgaagacatagaagcatccaaagaaagagagaagaaaagggaaaaggaagaaaagaagaggttagagctggagaaaaaggaacagaaagagaaagaaaagaaagaacaagaaataaagaagaaatttaaactaacaggccctattcaagtcatccatcttgcaaaagcttgttgtgatgtcaaaggaggaaagaatgaactgagcttcaagcaaggagagcaaattgaaatcatccgcatcacagacaacccagaaggaaaatggttgggcagaacagcaaggggttcatatggctatattaaaacaactgctgtagagattgactatgattctttgaaactgaaaaaagactctcttggtgccccttcaagacctattgaagatgaccaagaagtatatgatgatgttgcagagcaggatgatattagcagccacagtcagagtggaagtggagggatattccctccaccaccagatgatgacatttatgatgggattgaagaggaagatgctgatgatggctccacactacaggttcaagagaagagtaatacgtggtcctgggggattttgaagatgttaaagggaaaagatgacagaaagaaaagtatacgagagaaacctaaagtctctgactcagacaataatgaaggttcatctttccctgctcctcctaaacaattggacatgggagatgaagtttacgatgatgtggatacctctgatttccctgtttcatcagcagagatgagtcaaggaactaatgttggaaaagctaagacagaagaaaaggaccttaagaagctaaaaaagcaggaaaaagaagaaaaagacttcaggaaaaaatttaaatatgatggtgaaattagagtcctatattcaactaaagttacaacttccataacttctaaaaagtggggaaccagagatctacaggtaaaacctggtgaatctctagaagttatacaaaccacagatgacacaaaagttctctgcagaaatgaagaagggaaatatggttatgtccttcggagttacctagcggacaatgatggagagatctatgatgatattgctgatggctgcatctatgacaatgactagcactcaactttggtcattctgctgtgttcattaggtgccaatgtgaagtctggattttaattggcatgttattgggtatcaagaaaattaatgcacaaaaccacttattatcatttgttatgaaatcccaattatctttacaaagtgtttaaagtttgaacatagaaaataatctctctgcttaattgttaactcagaagactacattagtgagatgtaagaattattaaatattccatttccgctttggctacaattatgaagaagttgaaggtacttcttttagaccaccagtaaataatcctccttcaaaaaataaaaataaaagaaaaaggaaaatcattcaggaagaaatgacctgtctaaaaaaacctaaggaagaataataatataagaaaggaaatttaaaaacattccacaagaagaaaaattattgtttatacttctacttatggttatatcttatattctctattcaagtgacctgtcttttaaaaaggcagtgctgtcttacctcttgctagtgggttaaatgttttcaaaaattatagcagtagtagaagttttgtataaaatttgtccttatttgttaattgtatataaatgttaattatttgatacgaatgttatgcatttagtatgcacattgaagtctaaactgtagaagagtctaaaacaagttctctttttgcagattcacatactaatggtttaattctgtgctctgtttaaagtactattataactagagtagatctgaatgaggataaccctaaaatcatgaggaatggaagaatggaccttgaaactacctaggcttttatgcatggcacctctttataatgaagacactttttaaagtttttgtttttgtttcaattaccgctagatttttttttctctttttttaaaatccattttactggaaagttggccagcagagggagtagaaattattaaaattctagtgtttggattgggcccttctctaacagtacatactcattcccaaagcaatccaaaaacaaaatgtgaaccatttgggtttcaaatgttaagaacactaaatagcatgatttaaaaaatgaaaaatgctaacacccaagaaaagaagatattaagtgctttttaacaactcctagagtacaaaatgagtacatcataatgctggctcttctactaatgaaccatcgagtgatattgaataaattatttatcttctcagtttccttatctgtaaattacaatattagactaagtaagtttttccaactcttcactaccaattaccttaggcttttataatgctccgcctacttcagtcccatgtttcagaagcttttgtctattttttaaactcattgattaaataatgattaatgcattctccacattttaatattgcaaaggcccattggagtttctgaagtggctccacagaattgaaataatttcaaataactgtaaaggaactgaaaatcttcacagagatgaagtggggtttccattaggtgctttgaaatttgataacaaatcatcaacttccactggtcaatatatagattttgggtgtctgaggccccaagattagatgccactaatctccaaagattccctccaattatgaaatattttaatgtctacttttagagagcactagccagtatatgaccatgtgattaatttcttttcacactagataaaattacctggttcaaaagtggtttttgtttattaaatttggtaataaatatatataatacacagacaggatagtttttatgctgaagtttttggccagctttagtttgaggactccttgataagcttgctaaactttcagagtgccctgagacacttccagccatccctcctcctgccttcattggggcagacttgcattgcagtctgacagtaattttttttctgattgagaattatgtaaattcaatacaatgtcagtttttaaaagtcaaagttagatcaagagaatatttcagagttttggtttacacatcaagaaacagacacacatacctaggaaagatttacacaatagataatcatcttaatgtgaaagatatttgaagtattaattttaatatattaaatatgatttctgttatagtcttctgtatggaattttgtcacttaagatgagctgcaaataaataataccttcaatggaaaaaaaaaaaaaaaaaaaaaaaaaaaaaaaaaaaaaaaaaaaaaaaaa

SEQ ID NO: 17 - protein, 129 aa, Homo sapiens
MKILVALAVFFLVSTQLFAEEIGANDDLNYWSDWYDSDQIKEELPEPFEHLLQRIARRPKPQQFFGLMGKRDADSSIEKQVALLKALYGHGQISHKRHKTDSFVGLMGKRALNSVAYERSAMQNYERRR

SEQ ID NO: 18 - protein, 614 aa, Homo sapiens
MTTLDSNNNTGGVITYIGSSGSSPSRTSPESLYSDNSNGSFQSLTQGCPTYFPPSPTGSLTQDPARSFGSIPPSLSDDGSPSSSSSSSSSSSSFYNGSPPGSLQVAMEDSSRVSPSKSTSNITKLNGMVLLCKVCGDVASGFHYGVHACEGCKGFFRRSIQQNIQYKRCLKNENCSIVRINRNRCQQCRFKKCLSVGMSRDAVRFGRIPKREKQRMLAEMQSAMNLANNQLSSQCPLETSPTQHPTPGPMGPSPPPAPVPSPLVGFSQFPQQLTPPRSPSPEPTVEDVISQVARAHREIFTYAHDKLGSSPGNFNANHASGSPPATTPHRWENQGCPPAPNDNNTLAAQRHNEALNGLRQAPSSYPPTWPPGPAHHSCHQSNSNGHRLCPTHVYAAPEGKAPANSPRQGNSKNVLLACPMNMYPHGRSGRTVQEIWEDFSMSFTPAVREVVEFAKHIPGFRDLSQHDQVTLLKAGTFEVLMVRFASLFNVKDQTVMFLSRTTYSLQELGAMGMGDLLSAMFDFSEKLNSLALTEEELGLFTAVVLVSADRSGMENSASVEQLQETLLRALRALVLKNRPLETSRFTKLLLKLPDLRTLNNMHSEKLLSFRVDAQ

SEQ ID NO: 19 - protein, 359 aa, Homo sapiens
MPAHLLQDDISSSYTTTTTITAPPSRVLQNGGDKLETMPLYLEDDIRPDIKDDIYDPTYKDKEGPSPKVEYVWRNIILMSLLHLGALYGITLIPTCKFYTWLWGVFYYFVSALGITAGAHRLWSHRSYKARLPLRLFLIIANTMAFQNDVYEWARDHRAHHKFSETHADPHNSRRGFFFSHVGWLLVRKHPAVKEKGSTLDLSDLEAEKLVMFQRRYYKPGLLMMCFILPTLVPWYFWGETFQNSVFVATFLRYAVVLNATWLVNSAAHLFGYRPYDKNISPRENILVSLGAVGEGFHNYHHSFPYDYSASEYRWHINFTTFFIDCMAALGLAYDRKKVSKAAILARIKRTGDGNYKSG

SEQ ID NO: 20 - protein, 147 aa, Homo sapiens
MSQTRDLQGGKAFGLLKAQQEERLDEINKQFLDDPKYSSDEDLPSKLEGFKEKYMEFDLNGNGDIDIMSLKRMLEKLGVPKTHLELKKLIGEVSSGSGETFSYPDFLRMMLGKRSAILKMILMYEEKAREKEKPTGPPAKKAISELP

SEQ ID NO: 21 - protein, 261 aa, Homo sapiens
MSWKKALRIPGGLRAATVTLMLSMLSTPVAEGRDSPEDFVYQFKGMCYFTNGTERVRLVSRSIYNREEIVRFDSDVGEFRAVTLLGLPAAEYWNSQKDILERKRAAVDRVCRHNYQLELRTTLQRRVEPTVTISPSRTEALNHHNLLVCSVTDFYPAQIKVRWFRNDQEETAGVVSTPLIRNGDWTFQILVMLEMTPQRGDVYTCHVEHPSLQSPITVEWRAQSESAQSKMLSGIGGFVLGLIFLGLGLIIHHRSQKGLLH

SEQ ID NO: 22 - protein, 358 aa, Homo sapiens
MEQWDHFHNQQEDTDSCSESVKFDARSMTALLPPNPKNSPSLQEKLKSFKAALIALYLLVFAVLIPLIGIVAAQLLKWETKNCSVSSTNANDITQSLTGKGNDSEEEMRFQEVFMEHMSNMEKRIQHILDMEANLMDTEHFQNFSMTTDQRFNDILLQLSTLFSSVQGHGNAIDEISKSLISLNTTLLDLQLNIENLNGKIQENTFKQQEEISKLEERVYNVSAEIMAMKEEQVHLEQEIKGEVKVLNNITNDLRLKDWEHSQTLRNITLIQGPPGPPGEKGDRGPTGESGPRGFPGPIGPPGLKGDRGAIGFPGSRGLPGYAGRPGNSGPKGQKGEKGSGNTLRPVQLTDHIRAGPS

SEQ ID NO: 23 - protein, 451 aa, Homo sapiens
MEQWDHFHNQQEDTDSCSESVKFDARSMTALLPPNPKNSPSLQEKLKSFKAALIALYLLVFAVLIPLIGIVAAQLLKWETKNCSVSSTNANDITQSLTGKGNDSEEEMRFQEVFMEHMSNMEKRIQHILDMEANLMDTEHFQNFSMTTDQRFNDILLQLSTLFSSVQGHGNAIDEISKSLISLNTTLLDLQLNIENLNGKIQENTFKQQEEISKLEERVYNVSAEIMAMKEEQVHLEQEIKGEVKVLNNITNDLRLKDWEHSQTLRNITLIQGPPGPPGEKGDRGPTGESGPRGFPGPIGPPGLKGDRGAIGFPGSRGLPGYAGRPGNSGPKGQKGEKGSGNTLTPFTKVRLVGGSGPHEGRVEILHSGQWGTICDDRWEVRVGQVVCRSLGYPGVQAVHKAAHFGQGTGPIWLNEVFCFGRESSIEECKIRQWGTRACSHSEDAGVTCTL

SEQ ID NO: 24 - protein, 322 aa, Homo sapiens
MARCFSLVLLLTSIWTTRLLVQGSLRAEELSIQVSCRIMGITLVSKKANQQLNFTEAKEACRLLGLSLAGKDQVETALKASFETCSYGWVGDGFVVISRISPNPKCGKNGVGVLIWKVPVSRQFAAYCYNSSDTWTNSCIPEIITTKDPIFNTQTATQTTEFIVSDSTYSVASPYSTIPAPTTTPPAPASTSIPRRKKLICVTEVFMETSTMSTETEPFVENKAAFKNEAAGFGGVPTALLVLALLFFGAAAGLGFCYVKRYVKAFPFTNKNQQKEMIETKVVKEEKANDSNPNEESKKTDKNPEESKSPSKTTVRCLEAEV

SEQ ID NO: 25 - protein, 410 aa, Mus musculus
MFILLVLVTGVSAFTTPTVVHTGKVSESPITSEKPTVHGDNCQFRGREFKSELRLEGEPVVLRCPLAPHSDISSSSHSFLTWSKLDSSQLIPRDEPRMWVKGNILWILPAVQQDSGTYICTFRNASHCEQMSVELKVFKNTEASLPHVSYLQISALSTTGLLVCPDLKEFISSNADGKIQWYKGAILLDKGNKEFLSAGDPTRLLISNTSMDDAGYYRCVMTFTYNGQEYNITRNIELRVKGTTTEPIPVIISPLETIPASLGSRLIVPCKVFLGTGTSSNTIVWWLANSTFISAAYPRGRVTEGLHHQYSENDENYVEVSLIFDPVTREDLHTDFKCVASNPRSSQSLHTTVKEVSSTFSWSIALAPLSLIILVVGAIWMRRRCKRRAGKTYGLTKLRTDNQDFPSSPN

SEQ ID NO: 26 - protein, 274 aa, Homo sapiens
MVGRRALIVLAHSERTSFNYAMKEAAAAALKKKGWEVVESDLYAMNFNPIISRKDITGKLKDPANFQYPAESVLAYKEGHLSPDIVAEQKKLEAADLVIFQFPLQWFGVPAILKGWFERVFIGEFAYTYAAMYDKGPFRSKKAVLSITTGGSGSMYSLQGIHGDMNVILWPIQSGILHFCGFQVLEPQLTYSIGHTPADARIQILEGWKKRLENIWDETPLYFAPSSLFDLNFQAGFLMKKEVQDEEKNKKFGLSVGHHLGKSIPTDNQIKARK

SEQ ID NO: 27 - protein, 520 aa, Homo sapiens
MRNKKILKEDELLSETQQAAFHQIAMEPFEINVPKPKRRNGVNFSLAVVVIYLILLTAGAGLLVVQVLNLQARLRVLEMYFLNDTLAAEDSPSFSLLQSAHPGEHLAQGASRLQVLQAQLTWVRVSHEHLLQRVDNFTQNPGMFRIKGEQGAPGLQGHKGAMGMPGAPGPPGPPAEKGAKGAMGRDGATGPSGPQGPPGVKGEAGLQGPQGAPGKQGATGTPGPQGEKGSKGDGGLIGPKGETGTKGEKGDLGLPGSKGDRGMKGDAGVMGPPGAQGSKGDFGRPGPPGLAGFPGAKGDQGQPGLQGVPGPPGAVGHPGAKGEPGSAGSPGRAGLPGSPGSPGATGLKGSKGDTGLQGQQGRKGESGVPGPAGVKGEQGSPGLAGPKGAPGQAGQKGDQGVKGSSGEQGVKGEKGERGENSVSVRIVGSSNRGRAEVYYSGTWGTICDDEWQNSDAIVFCRMLGYSKGRALYKVGAGTGQIWLDNVQCRGTESTLWSCTKNSWGHHDCSHEEDAGVECSV

SEQ ID NO: 28 - protein, 951 aa, Homo sapiens
MDGRWQCSCWAWFLLVLAVVAGDTVSTGSTDNSPTSNSLEGGTDATAFWWGEWTKWTACSRSCGGGVTSQERHCLQQRRKSVPGPGNRTCTGTSKRYQLCRVQECPPDGRSFREEQCVSFNSHVYNGRTHQWKPLYPDDYVHISSKPCDLHCTTVDGQRQLMVPARDGTSCKLTDLRGVCVSGKCEPIGCDGVLFSTHTLDKCGICQGDGSSCTHVTGNYRKGNAHLGYSLVTHIPAGARDIQIVERKKSADVLALADEAGYYFFNGNYKVDSPKNFNIAGTVVKYRRPMDVYETGIEYIVAQGPTNQGLNVMVWNQNGKSPSITFEYTLLQPPHESRPQPIYYGFSESAESQGLDGAGLMGFVPHNGSLYGQASSERLGLDNRLFGHPGLDMELGPSQGQETNEVCEQAGGGACEGPPRGKGFRDRNVTGTPLTGDKDDEEVDTHFASQEFFSANAISDQLLGAGSDLKDFTLNETVNSIFAQGAPRSSLAESFFVDYEENEGAGPYLLNGSYLELSSDRVANSSSEAPFPNVSTSLLTSAGNRTHKARTRPKARKQGVSPADMYRWKLSSHEPCSATCTTGVMSAYAMCVRYDGVEVDDSYCDALTRPEPVHEFCAGRECQPRWETSSWSECSRTCGEGYQFRVVRCWKMLSPGFDSSVYSDLCEAAEAVRPEERKTCRNPACGPQWEMSEWSECTAKCGERSVVTRDIRCSEDEKLCDPNTRPVGEKNCTGPPCDRQWTVSDWGPCSGSCGQGRTIRHVYCKTSDGRVVPESQCQMETKPLAIHPCGDKNCPAHWLAQDWERCNTTCGRGVKKRLVLCMELANGKPQTRSGPECGLAKKPPEESTCFERPCFKWYTSPWSECTKTCGVGVRMRDVKCYQGTDIVRGCDPLVKPVGRQACDLQPCPTEPPDDSCQDQPGTNCALAIKVNLCGHWYYSKACCRSCRPPHS

SEQ ID NO: 29 - protein, 943 aa, Homo sapiens
MTQRSIAGPICNLKFVTLLVALSSELPFLGAGVQLQDNGYNGLLIAINPQVPENQNLISNIKEMITEASFYLFNATKRRVFFRNIKILIPATWKANNNSKIKQESYEKANVIVTDWYGAHGDDPYTLQYRGCGKEGKYIHFTPNFLLNDNLTAGYGSRGRVFVHEWAHLRWGVFDEYNNDKPFYINGQNQIKVTRCSSDITGIFVCEKGPCPQENCIISKLFKEGCTFIYNSTQNATASIMFMQSLSSVVEFCNASTHNQEAPNLQNQMCSLRSAWDVITDSADFHHSFPMNGTELPPPPTFSLVQAGDKVVCLVLDVSSKMAEADRLLQLQQAAEFYLMQIVEIHTFVGIASFDSKGEIRAQLHQINSNDDRKLLVSYLPTTVSAKTDISICSGLKKGFEVVEKLNGKAYGSVMILVTSGDDKLLGNCLPTVLSSGSTIHSIALGSSAAPNLEELSRLTGGLKFFVPDISNSNSMIDAFSRISSGTGDIFQQHIQLESTGENVKPHHQLKNTVTVDNTVGNDTMFLVTWQASGPPEIILFDPDGRKYYTNNFITNLTFRTASLWIPGTAKPGHWTYTLNNTHHSLQALKVTVTSRASNSAVPPATVEAFVERDSLHFPHPVMIYANVKQGFYPILNATVTATVEPETGDPVTLRLLDDGAGADVIKNDGIYSRYFFSFAANGRYSLKVHVNHSPSISTPAHSIPGSHAMYVPGYTANGNIQMNAPRKSVGRNEEERKWGFSRVSSGGSFSVLGVPAGPHPDVFPPCKIIDLEAVKVEEELTLSWTAPGEDFDQGQATSYEIRMSKSLQNIQDDFNNAILVNTSKRNPQQAGIREIFTFSPQISTNGPEHQPNGETHESHRIYVAIRAMDRNSLQSAVSNIAQAPLFIPPNSDPVPARDYLILKGVLTAMGLIGIICLIIVVTHHTLSRKKRADKKENGTKLL

SEQ ID NO: 30 - protein, 753 aa, Homo sapiens
MERRAWSLQCTAFVLFCAWCALNSAKAKRQFVNEWAAEIPGGPEAASAIAEELGYDLLGQIGSLENHYLFKHKNHPRRSRRSAFHITKRLSDDDRVIWAEQQYEKERSKRSALRDSALNLFNDPMWNQQWYLQDTRMTAALPKLDLHVIPVWQKGITGKGVVITVLDDGLEWNHTDIYANYDPEASYDFNDNDHDPFPRYDPTNENKHGTRCAGEIAMQANNHKCGVGVAYNSKVGGIRMLDGIVTDAIEASSIGFNPGHVDIYSASWGPNDDGKTVEGPGRLAQKAFEYGVKQGRQGKGSIFVWASGNGGRQGDNCDCDGYTDSIYTISISSASQQGLSPWYAEKCSSTLATSYSSGDYTDQRITSADLHNDCTETHTGTSASAPLAAGIFALALEANPNLTWRDMQHLVVWTSEYDPLANNPGWKKNGAGLMVNSRFGFGLLNAKALVDLADPRTWRSVPEKKECVVKDNDFEPRALKANGEVIIEIPTRACEGQENAIKSLEHVQFEATIEYSRRGDLHVTLTSAAGTSTVLLAERERDTSPNGFKNWDFMSVHTWGENPIGTWTLRITDMSGRIQNEGRIVNWKLILHGTSSQPEHMKQPRVYTSYNTVQNDRRGVEKMVDPGEEQPTQENPKENTLVSKSPSSSSVGGRRDELEEGAPSQAMLRLLQSAFSKNSPPKQSPKKSPSAKLNIPYENFYEALEKLNKPSQLKDSEDSLYNDYVDVFYNTKPYKHRDDRLLQALVDILNEEN

SEQ ID NO: 31 - protein, 222 aa, Homo sapiens
MKLPLLLALLFGAVSALHLRSETSTFETPLGAKTLPEDEETPEQEMEETPCRELEEEEEWGSGSEDASKKDGAVESISVPDMVDKNLTCPEEEDTVKVVGIPGCQTCRYLLVRSLQTFSQAWFTCRRCYRGNLVSIHNFNINYRIQCSVSALNQGQVWIGGRITGSGRCRRFQWVDGSRWNFAYWAAHQPWSRGGHCVALCTRGGHWRRAHCLRRLPFICSY

SEQ ID NO: 32 - protein, 829 aa, Homo sapiens
MAKYNTGGNPTEDVSVNSRPFRVTGPNSSSGIQARKNLFNNQGNASPPAGPSNVPKFGSPKPPVAVKPSSEEKPDKEPKPPFLKPTGAGQRFGTPASLTTRDPEAKVGFLKPVGPKPINLPKEDSKPTFPWPPGNKPSLHSVNQDHDLKPLGPKSGPTPPTSENEQKQAFPKLTGVKGKFMSASQDLEPKPLFPKPAFGQKPPLSTENSHEDESPMKNVSSSKGSPAPLGVRSKSGPLKPAREDSENKDHAGEISSLPFPGVVLKPAASRGGPGLSKNGEEKKEDRKIDAAKNTFQSKINQEELASGTPPARFPKAPSKLTVGGPWGQSQEKEKGDKNSATPKQKPLPPLFTLGPPPPKPNRPPNVDLTKFHKTSSGNSTSKGQTSYSTTSLPPPPPSHPASQPPLPASHPSQPPVPSLPPRNIKPPFDLKSPVNEDNQDGVTHSDGAGNLDEEQDSEGETYEDIEASKEREKKREKEEKKRLELEKKEQKEKEKKEQEIKKKFKLTGPIQVIHLAKACCDVKGGKNELSFKQGEQIEIIRITDNPEGKWLGRTARGSYGYIKTTAVEIDYDSLKLKKDSLGAPSRPIEDDQEVYDDVAEQDDISSHSQSGSGGIFPPPPDDDIYDGIEEEDADDGSTLQVQEKSNTWSWGILKMLKGKDDRKKSIREKPKVSDSDNNEGSSFPAPPKQLDMGDEVYDDVDTSDFPVSSAEMSQGTNVGKAKTEEKDLKKLKKQEKEEKDFRKKFKYDGEIRVLYSTKVTTSITSKKWGTRDLQVKPGESLEVIQTTDDTKVLCRNEEGKYGYVLRSYLADNDGEIYDDIADGCIYDND

SEQ ID NO: 33 - DNA, 1403 bp, Homo sapiens
gcctgtctgcattctactatataaagcagcagagacgttgactagcgcatatttgctaagagcaccatgcgcgcagcagccatctccactccaaagttagacaaaatgccaggaatgttcttctctgctaacccaaaggaattgaaaggaaccactcattcacttctagacgacaaaatgcaaaaaaggaggccaaagacttttggaatggatatgaaagcatacctgagatctatgatcccacatctggaatctggaatgaaatcttccaagtccaaggatgtactttctgctgctgaagtaatgcaatggtctcaatctctggaaaaacttcttgccaaccaaactggtcaaaatgtctttggaagtttcctaaagtctgaattcagtgaggagaatattgagttctggctggcttgtgaagactataagaaaacagagtctgatcttttgccctgtaaagcagaagagatatataaagcatttgtgcattcagatgctgctaaacaaatcaatattgacttccgcactcgagaatctacagccaagaagattaaagcaccaacccccacgtgttttgatgaagcacaaaaagtcatatatactcttatggaaaaggactcttatcccaggttcctcaaatcagatatttacttaaatcttctaaatgacctgcaggctaatagcctaaagtgactggtccctggctgaagggaattaacagatagtatcaagcgcagaaggaatgtgccagtatggctccctgggtgaacagcttggccttttttgggtgtcttgacaggccaagaagaacaaatgactcagaatggattaacatgaaagttatccaggcgcagagttgaagaagcataagcaagacaaaaacagagagaccgcagaaggaggaagatactgtggtactgtcataaaaaacagtggagctctgtattagaaagcccctcagaactgggaaggccaggtaactctagttacacagaaactgtgactaaagtctatgaaactgattacaacagactgtaagaatcaaagtcaactgacatctatgctacatattattatatagtttgtactgagctattgaagtcccattaacttaaagtatatgttttcaaattgccattgctactattgcttgtcggtgttattttattttattgtttttgactttggaagagatgaactgtgtatttaacttaagctattgctcttaaaaccagggagtcagaatatatttgtaagttaaatcattggtgctaataataaatgtggattttgtattaaaatatatagaagcaatttctgtttacatgtccttgctacttttaaaaacttgcatttattcctcagattttaaaaataaataaataattcatttaagattc

SEQ ID NO: 34 - DNA, 3726 bp, Homo sapiens
cagcgctgctccccgggcgctcctccccgggcgctcctccccaggcctcccgggcgcttggatcccggccatctccgcacccttcaagtgggtgtgggtgatttcctggcggggggagcagccaggtgagcccaagatgctgctgcgctcgaagcctgcgctgccgccgccgctgatgctgctgctcctggggccgctgggtcccctctcccctggcgccctgccccgacctgcgcaagcacaggacgtcgtggacctggacttcttcacccaggagccgctgcacctggtgagcccctcgttcctgtccgtcaccattgacgccaacctggccacggacccgcggttcctcatcctcctgggttctccaaagcttcgtaccttggccagaggcttgtctcctgcgtacctgaggtttggtggcaccaagacagacttcctaattttcgatcccaagaaggaatcaacctttgaagagagaagttactggcaatctcaagtcaaccaggatatttgcaaatatggatccatccctcctgatgtggaggagaagttacggttggaatggccctaccaggagcaattgctactccgagaacactaccagaaaaagttcaagaacagcacctactcaagaagctctgtagatgtgctatacacttttgcaaactgctcaggactggacttgatctttggcctaaatgcgttattaagaacagcagatttgcagtggaacagttctaatgctcagttgctcctggactactgctcttccaaggggtataacatttcttgggaactaggcaatgaacctaacagtttccttaagaaggctgatattttcatcaatgggtcgcagttaggagaagattttattcaattgcataaacttctaagaaagtccaccttcaaaaatgcaaaactctatggtcctgatgttggtcagcctcgaagaaagacggctaagatgctgaagagcttcctgaaggctggtggagaagtgattgattcagttacatggcatcactactatttgaatggacggactgctaccagggaagattttctaaaccctgatgtattggacatttttatttcatctgtgcaaaaagttttccaggtggttgagagcaccaggcctggcaagaaggtctggttaggagaaacaagctctgcatatggaggcggagcgcccttgctatccgacacctttgcagctggctttatgtggctggataaattgggcctgtcagcccgaatgggaatagaagtggtgatgaggcaagtattctttggagcaggaaactaccatttagtggatgaaaacttcgatcctttacctgattattggctatctcttctgttcaagaaattggtgggcaccaaggtgttaatggcaagcgtgcaaggttcaaagagaaggaagcttcgagtataccttcattgcacaaacactgacaatccaaggtataaagaaggagatttaactctgtatgccataaacctccataatgtcaccaagtacttgcggttaccctatcctttttctaacaagcaagtggataaataccttctaagacctttgggacctcatggattactttccaaatctgtccaactcaatggtctaactctaaagatggtggatgatcaaaccttgccacctttaatggaaaaacctctccggccaggaagttcactgggcttgccagctttctcatatagtttttttgtgataagaaatgccaaagttgctgcttgcatctgaaaataaaatatactagtcctgacactgaatttttcaagtatactaagagtaaagcaactcaagttataggaaaggaagcagataccttgcaaagcaactagtgggtgcttgagagacactgggacactgtcagtgctagatttagcacagtattttgatctcgctaggtagaacactgctaataataatagctaataataccttgttccaaatactgcttagcattttgcatgttttacttttatctaaagttttgttttgttttattatttatttatttatttattttgtgacggagagagattccatctcaaaaaaacaagttattaaaaatgtatatgaatgctcctaatatggtcaggaagcaaggaagcgaaggatatattatgagttttaagaaggtgcttagctgtatatttatctttcaaaatgtattagaagattttagaattctttccttcatgtgccatctctacaggcacccatcagaaaaagcatactgccgttaccgtgaaactggttgtaaaagagaaactatctatttgcaccttaaaagacagctagattttgctgattttcttctttcggttttctttgtcagcaataatatgtgagaggacagattgttagatatgatagtataaaaaatggttaatgacaattcagaggcgaggagattctgtaaacttaaaattactataaatgaaattgatttgtcaagaggataaattttagaaaacacccaataccttataactgtctgttaatgcttgctttttctctacctttcttccttgtttcagttgggaagcttttggctgcaagtaacagaaactcctaattcaaatggcttaagcaataaggaaatgtatattcccacataactagacgttcaaacaggccaggctccagcacttcagtacgtcaccaggggatctgggttcttcccagctctctgctctgccatctttagcgctggcttcattctcagactctggtagcatgatggctgtagctgtttcatgggccccttcaaacctcatagcaaccagaggaagaaaatgagccattttttgagtctccttcatagacttgaataactctttttcagagcttctcacagcaaacctctcctcatgtctcctcatgtcttattgttcagaaatgggtaatgtggccatttcaccagtcactgccaacaacaacgaggttcctataattgtctctgagtaaccctttggaatggagagggtgttggtcagtctacaaactgaacactgcagttctgcgctttttaccagtgaaaaaatgtaattattttcccctcttaaggattaatattcttcaaatgtatgcctgttatggatatagtatctttaaaattttttattttaatagctttaggggtacacactttttgcttacaggggtgaattgtgtagtggtgaagactcggcttttaatgtacttgtcacctgagtgatgtacattgtacccaataggtaatttttcatccattaccctccttccgccctcttcccttctgagtctccaacatcccttataccactgtgtatgttcttgtgtacctacagctaagcttccacttataagtgagaacatgcagtatttggttttccattcctgagttacttcccttaggataacagcccccagttccgtccaagttgctgcaaaatacattattcttctttatggctgagtaatagtccatggtacatatataccacattttctttatccacttatcagttgatggacacttaggttaattccattcaatttcattcaatttaagtatatttgtaaggagctaaagctgaaaattaaattttagatctttcaatactcttaaattttatatgtaagtggtttttatattttcacatttgaaataaagtaatttttataaccttgaaaaaaaaaaaaaaaaaaaa

SEQ ID NO: 35 - DNA, 2260 bp, Homo sapiens
gggggtatttgtttcactgctttcaaccgcctgtgctggaggctcagaataagtcaatgggaggaggatttcagtcacagcagcaagcaagtctagtgaacagataagatgacatgctcagcaaaataacaacgaaaccagagggggaactctctggcatgcaagttcaaacacgactctacaactacggcagaaaaagagagagagagaaactaaaaatatatatatatcctatttttttcacagctatcagtttctttcactgagctttcctaaatttaagcctctagaaaataataaatacttggatatcttacctacaaacatggacagatgtgtgtatgcgctcattttagagaacttgaatttttttttttaaaggaaggtgtcaactttggcttttgagtgtttggcatggttacaatgccttaaaaaaacagatgagcagcttagctactaaccatgctgaccactgttcggaacgggattgaatcacagaaaaacagcaaatggctctctcttacagagtgtctgaacttcaaagcacaattcctgagcacattttgcagagcacctttgttcacgttatctcttctaactggtctggattacagacagaatcaataccagaggaaatgaaacagattgttgaggaacagggaaataaactgcactgggcagctcttctgatactcatggtgataatacccacaattggtggaaatacccttgttattctggctgtttcactggagaagaagctgcagtatgctactaattactttctaatgtccttggcggtggctgatttgctggttggattgtttgtgatgccaattgccctcttgacaataatgtttgaggctatgtggcccctcccacttgttctatgtcctgcctggttatttcttgacgttctcttttcaaccgcatccatcatgcatctctgtgccatttcagtggatcgttacatagccatcaaaaagccaatccaggccaatcaatataactcacgggctacagcattcatcaagattacagtggtgtggttaatttcaataggcattgccattccagtccctattaaagggatagagactgatgtggacaacccaaacaatatcacttgtgtgctgacaaaggaacgttttggcgatttcatgctctttggctcactggctgccttcttcacacctcttgcaattatgattgtcacctactttctcactatccatgctttacagaagaaggcttacttagtcaaaaacaagccacctcaacgcctaacatggttgactgtgtctacagttttccaaagggatgaaacaccttgctcgtcaccggaaaaggtggcaatgctggatggttctcgaaaggacaaggctctgcccaactcaggtgatgaaacacttatgcgaagaacatccacaattgggaaaaagtcagtgcagaccatttccaacgaacagagagcctcaaaggtcctagggattgtgtttttcctctttttgcttatgtggtgtcccttctttattacaaatataactttagttttatgtgattcctgtaaccaaactactctccaaatgctcctggagatatttgtgtggataggctatgtttcctcaggagtgaatcctttggtctacaccctcttcaataagacatttcgggatgcatttggccgatatatcacctgcaattaccgggccacaaagtcagtaaaaactctcagaaaacgctccagtaagatctacttccggaatccaatggcagagaactctaagtttttcaagaaacatggaattcgaaatgggattaaccctgccatgtaccagagtccaatgaggctccgaagttcaaccattcagtcttcatcaatcattctactagatacgcttctcctcactgaaaatgaaggtgacaaaactgaagagcgagttagttatgtatagcagaactggcagttgtcatcaaacataatgatgagtaagatgatgaatgagatgtaaatgtgccaagaatatattatataaagaattttatgtcatatatcaaatcatctctttaacctaagatgtaagtattaagaatatctaattttcctaatttggacaagattattccatgaggaaaataattttatatagctacaaatgaaaacaatccagcactctggttaaattttaaggtattcgaatgaaataaagtcaaatcaataaatttcaggccaaaaaaaaaaaaaaaaaaaaaaaaaaaa

SEQ ID NO: 36 - DNA, 3316 bp, Homo sapiens
gtaggggtaggagggggccggcggagtttccctccccgcccagcggccctgggcgggcttttcggctgcttctcataagcaggtggtttcgtttctccggcacaggtaggtttctctggcaccgattcggggcctgcccggacttcgccgcacgctgcagaacctcgcccagcgcccaccatgccccggcagctcagcgcggcggccgcgctcttcgcgtccctggccgtaattttgcacgatggcagtcaaatgagagcaaaagcatttccagaaaccagagattattctcaacctactgcagcagcaacagtacaggacataaaaaaacctgtccagcaaccagctaagcaagcacctcaccaaactttagcagcaagattcatggatggtcatatcacctttcaaacagcggccacagtaaaaattccaacaactaccccagcaactacaaaaaacactgcaaccaccagcccaattacctacaccctggtcacaacccaggccacacccaacaactcacacacagctcctccagttactgaagttacagtcggccctagcttagccccttattcactgccacccaccatcaccccaccagctcatacaactggaaccagttcatcaaccgtcagccacacaactgggaacaccactcaacccagtaaccagaccacccttccagcaactttatcgatagcactgcacaaaagcacaaccggtcagaagcctgttcaacccacccatgccccaggaacaacggcagctgcccacaataccacccgcacagctgcacctgcctccacggttcctgggcccacccttgcacctcagccatcgtcagtcaagactggaatttatcaggttctaaacggaagcagactctgtataaaagcagagatggggatacagctgattgttcaagacaaggagtcggttttttcacctcggagatacttcaacatcgaccccaacgcaacgcaagcctctgggaactgtggcacccgaaaatccaaccttctgttgaattttcagggcggatttgtgaatctcacatttaccaaggatgaagaatcatattatatcagtgaagtgggagcctatttgaccgtctcagatccagagacaatttaccaaggaatcaaacatgcggtggtgatgttccagacagcagtcgggcattccttcaagtgcgtgagtgaacagagcctccagttgtcagcccacctgcaggtgaaaacaaccgatgtccaacttcaagcctttgattttgaagatgaccactttggaaatgtggatgagtgctcgtctgactacacaattgtgcttcctgtgattggggccatcgtggttggtctctgccttatgggtatgggtgtctataaaatccgcctaaggtgtcaatcatctggataccagagaatctaattgttgcccggggggaatgaaaataatggaatttagagaactctttcatcccttccaggatggatgttgggaaattccctcagagtgtgggtccttcaaacaatgtaaaccaccatcttctattcaaatgaagtgagtcatgtgtgatttaagttcaggcagcacatcaatttctaaatactttttgtttattttatgaaagatatagtgagctgtttattttctagtttcctttagaatattttagccactcaaagtcaacatttgagatatgttgaattaacataatatatgtaaagtagaataagccttcaaattataaaccaagggtcaattgtaactaatactactgtgtgtgcattgaagattttattttacccttgatcttaacaaagcctttgctttgttatcaaatggactttcagtgcttttactatctgtgttttatggtttcatgtaacatacatattcctggtgtagcacttaactccttttccactttaaatttgtttttgttttttgagacggagtttcactcttgtcacccaggctggagtacagtggcacgatctcggcttatggcaacctccgcctcccgggttcaagtgattctcctgcttcagcttcccgagtagctgggattacaggcacacactaccacgcctggctaatttttgtatttttattatagacggggtttcaccatgttggccagactggtcttgaactcttgacctcaggtgatccacccacctcagcctcccaaagtgctgggattacaggcatgagccattgcgcccggccttaaatgttttttttaatcatcaaaaagaacaacatatctcaggttgtctaagtgtttttatgtaaaaccaacaaaaagaacaaatcagcttatattttttatcttgatgactcctgctccagaatcgctagactaagaattaggtggctacagatggtagaactaaacaataagcaagagacaataataatggcccttaattattaacaaagtgccagagtctaggctaagcactttatctatatctcatttcattctcacaacttataggtgaatgagtaaactgagacttaagggaactgaatcacttaaatgtcacctggctaactgatggcagagccagagcttgaattcatgttggtctgacatcaaggtctttggtcttctccctacaccaagttacctacaagaacaatgacaccacactctgcctgaaggctcacacctcataccagcatacgctcaccttacagggaaatgggtttatccaggatcatgagacattagggtagatgaaaggagagctttgcagataacaaaatagcctatccttaataaatcctccactctctggaaggagactgaggggctttgtaaaacattagtcagttgctcatttttatgggattgcttagctgggctgtaaagatgaaggcatcaaataaactcaaagtatttttaaatttttttgataatagagaaacttcgctaaccaactgttctttcttgagtgtatagccccatcttgtggtaacttgctgcttctgcacttcatatccatatttcctattgttcactttattctgtagagcagcctgccaagaattttatttctgctgttttttttgctgctaaagaaaggaactaagtcaggatgttaacagaaaagtccacataaccctagaattcttagtcaaggaataattcaagtcagcctagagaccatgttgactttcctcatgtgtttccttatgactcagtaagttggcaaggtcctgactttagtcttaataaaacattgaattgtagtaaaggtttttgtaataaaaacttactttggaaaaaaaaaaaaaaaaaaaa

SEQ ID NO: 37 - DNA, 523 bp, Homo sapiens
ctcctggttcaaaagcagctaaaccaaaagaagcctccagacagccctgagatcacctaaaaagctgctaccaagacagccacgaagatcctaccaaaatgaagcgcttcctcttcctcctactcaccatcagcctcctggttatggtacagatacaaactggactctcaggacaaaacgacaccagccaaaccagcagcccctcagcatccagcaacataagcggaggcattttccttttcttcgtggccaatgccataatccacctcttctgcttcagttgaggtgacacgtctcagccttagccctgtgccccctgaaacagctgccaccatcactcgcaagagaatcccctccatctttgggaggggttgatgccagacatcaccaggttgtagaagttgacaggcagtgccatgggggcaacagccaaaataggggggtaatgatgtaggggccaagcagtgcccagctgggggtcaataaagttacccttgtacttgcaaaaaaaaaaaaaaaaaaa

SEQ ID NO: 38 - DNA, 6133 bp, Homo sapiens
tgcatgaagacaaaaggtcctgtgctcacctgggacccttctggacgttgccctgtgtacctcttcgactgcctgttcatctacgacgaaccccgggtattgaccccagacaacaatgccacttcatattggggacttcgtctgggattccaaggtgcattcattgcaaagttccttaaatattttctcactgcttcctactaaaggacggacagagcatttgttcttcagccacatactttccttccactggccagcattctcctctattagactagaactgtggataaacctcagaaaatggccacccagcagaaagcctctgacgagaggatctcccagtttgatcacaatttgctgccagagctgtctgctcttctgggcctagatgcagttcagttggcaaaggaactagaagaagaggagcagaaggagcgagcaaaaatgcagaaaggctacaactctcaaatgcgcagtgaagcaaaaaggttaaagacttttgtgacttatgagccgtacagctcatggataccacaggagatggcggccgctgggttttacttcactggggtaaaatctgggattcagtgcttctgctgtagcctaatcctctttggtgccggcctcacgagactccccatagaagaccacaagaggtttcatccagattgtgggttccttttgaacaaggatgttggtaacattgccaagtacgacataagggtgaagaatctgaagagcaggctgagaggaggtaaaatgaggtaccaagaagaggaggctagacttgcgtccttcaggaactggccattttatgtccaagggatatccccttgtgtgctctcagaggctggctttgtctttacaggtaaacaggacacggtacagtgtttttcctgtggtggatgtttaggaaattgggaagaaggagatgatccttggaaggaacatgccaaatggttccccaaatgtgaatttcttcggagtaagaaatcctcagaggaaattacccagtatattcaaagctacaagggatttgttgacataacgggagaacattttgtgaattcctgggtccagagagaattacctatggcatcagcttattgcaatgacagcatctttgcttacgaagaactacggctggactcttttaaggactggccccgggaatcagctgtgggagttgcagcactggccaaagcaggtcttttctacacaggtataaaggacatcgtccagtgcttttcctgtggagggtgtttagagaaatggcaggaaggtgatgacccattagacgatcacaccagatgttttcccaattgtccatttctccaaaatatgaagtcctctgcggaagtgactccagaccttcagagccgtggtgaactttgtgaattactggaaaccacaagtgaaagcaatcttgaagattcaatagcagttggtcctatagtgccagaaatggcacagggtgaagcccagtggtttcaagaggcaaagaatctgaatgagcagctgagagcagcttataccagcgccagtttccgccacatgtctttgcttgatatctcttccgatctggccacggaccacttgctgggctgtgatctgtctattgcttcaaaacacatcagcaaacctgtgcaagaacctctggtgctgcctgaggtctttggcaacttgaactctgtcatgtgtgtggagggtgaagctggaagtggaaagacggtcctcctgaagaaaatagcttttctgtgggcatctggatgctgtcccctgttaaacaggttccagctggttttctacctctcccttagttccaccagaccagacgaggggctggccagtatcatctgtgaccagctcctagagaaagaaggatctgttactgaaatgtgcatgaggaacattatccagcagttaaagaatcaggtcttattccttttagatgactacaaagaaatatgttcaatccctcaagtcataggaaaactgattcaaaaaaaccacttatcccggacctgcctattgattgctgtccgtacaaacagggccagggacatccgccgatacctagagaccattctagagatcaaagcatttcccttttataatactgtctgtatattacggaagctcttttcacataatatgactcgtctgcgaaagtttatggtttactttggaaagaaccaaagtttgcagaagatacagaaaactcctctctttgtggcggcgatctgtgctcattggtttcagtatccttttgacccatcctttgatgatgtggctgttttcaagtcctatatggaacgcctttccttaaggaacaaagcgacagctgaaattctcaaagcaactgtgtcctcctgtggtgagctggccttgaaagggtttttttcatgttgctttgagtttaatgatgatgatctcgcagaagcaggggttgatgaagatgaagatctaaccatgtgcttgatgagcaaatttacagcccagagactaagaccattctaccggtttttaagtcctgccttccaagaatttcttgcggggatgaggctgattgaactcctggattcagataggcaggaacatcaagatttgggactgtatcatttgaaacaaatcaactcacccatgatgactgtaagcgcctacaacaattttttgaactatgtctccagcctcccttcaacaaaagcagggcccaaaattgtgtctcatttgctccatttagtggataacaaagagtcattggagaatatatctgaaaatgatgactacttaaagcaccagccagaaatttcactgcagatgcagttacttaggggattgtggcaaatttgtccacaagcttacttttcaatggtttcagaacatttactggttcttgccctgaaaactgcttatcaaagcaacactgttgctgcgtgttctccatttgttttgcaattccttcaagggagaacactgactttgggtgcgcttaacttacagtactttttcgaccacccagaaagcttgtcattgttgaggagcatccacttcccaatacgaggaaataagacatcacccagagcacatttttcagttctggaaacatgttttgacaaatcacaggtgccaactatagatcaggactatgcttctgcctttgaacctatgaatgaatgggagcgaaatttagctgaaaaagaggataatgtaaagagctatatggatatgcagcgcagggcatcaccagaccttagtactggctattggaaactttctccaaagcagtacaagattccctgtctagaagtcgatgtgaatgatattgatgttgtaggccaggatatgcttgagattctaatgacagttttctcagcttcacagcgcatcgaactccatttaaaccacagcagaggctttatagaaagcatccgcccagctcttgagctgtctaaggcctctgtcaccaagtgctccataagcaagttggaactcagcgcagccgaacaggaactgcttctcaccctgccttccctggaatctcttgaagtctcagggacaatccagtcacaagaccaaatctttcctaatctggataagttcctgtgcctgaaagaactgtctgtggatctggagggcaatataaatgttttttcagtcattcctgaagaatttccaaacttccaccatatggagaaattattgatccaaatttcagctgagtatgatccttccaaactagtaaaattaattcaaaattctccaaaccttcatgttttccatctgaagtgtaacttcttttcggattttgggtctctcatgactatgcttgtttcctgtaagaaactcacagaaattaagttttcggattcattttttcaagccgtcccatttgttgccagtttgccaaattttatttctctgaagatattaaatcttgaaggccagcaatttcctgatgaggaaacatcagaaaaatttgcctacattttaggttctcttagtaacctggaagaattgatccttcctactggggatggaatttatcgagtggccaaactgatcatccagcagtgtcagcagcttcattgtctccgagtcctctcatttttcaagactttgaatgatgacagcgtggtggaaattgccaaagtagcaatcagtggaggtttccagaaacttgagaacctaaagctttcaatcaatcacaagattacagaggaaggatacagaaatttctttcaagcactggacaacatgccaaacttgcaggagttggacatctccaggcatttcacagagtgtatcaaagctcaggccacaacagtcaagtctttgagtcaatgtgtgttacgactaccaaggctcattagactgaacatgttaagttggctcttggatgcagatgatattgcattgcttaatgtcatgaaagaaagacatcctcaatctaagtacttaactattctccagaaatggatactgccgttctctccaatcattcagaaataaaagattcagctaaaaactgctgaatcaataatttgtcttggggcatattgaggatgtaaaaaaagttgttgattaatgctaaaaaccaaattatccaaaattattttattaaatattgcatacaaaagaaaatgtgtaaggcttgctaaaaaacaaaacaaaacaaaacacagtcctgcatactcaccaccaagctcaagaaataaatcatcaccaatacctttgaggtccctgagtaatccaccccagctaaaggcaaacccttcaatcaagtttatacagcaaaccctccattgtccatggtcaacagggaaggggttggggacaggtctgccaatctatctaaaagccacaatatggaagaagtattcaatttatataataaatggctaacttaacggttgaatcactttcatacatggatgaaacgggtttaacacaggatccacatgaatcttctgtgggccaagagatgttccttaatccttgtagaacctgttttctatattgaactagctttggtacagtagagttaacttactttccatttatccactgccaatataaagaggaaacaggggttagggaaaaatgacttcattccagaggcttctcagagttcaacatatgctataatttagaattttcttatgaatccactctacttgggtagaaaatattttatctctagtgattgcatattatttccatatcatagtatttcatagtattatatttgatatgagtgtctatatcaatgtcagtgtccagaatttcgttcctaccagttaagtagttttctgaacggccagaagaccattcgaaattcatgatactactataagttggtaaacaaccatacttttatcctcatttttattctcactaagaaaaaagtcaactcccctccccttgcccaagtatgaaatatagggacagtatgtatggtgtggtctcatttgtttagaaaaccacttatgactgggtgcggtggctcacacctgtaatcccagcactttgggaggctgaggcgggcgaatcatttgaggtgaggaattcgagaccagcctggccagcatggtgaaaccccatctctactaaaaatacaaaaattagccaggtgtggtggcacatgcctgtagtcccagccactagggcggctgagacgcaagacttgcttgaacccgggaggcagaggttgcagtgagccaagatggcgccactgcattccagcctgggcaacagagcaagaccctgtctgtctcaaaacaaaaaacaaaaccacttatattgctagctacattaagaatttctgaatatgttactgagcttgcttgtggtaaccatttataatatcagaaagtatatgtacaccaaaacatgttgaacatccatgttgtacaactgaaatataaataattttgtcaattatacctaaataaaactggaaaaaaatttctggaagtttatatctaaaaatgttaatagtgcgtacctctaggaagtgggcctggaagccattcttacttttcagtctctcccattctgtactgttttttgttttactttcgtgcctgcattatttttctatttaaaacaaaaataaatctagtttagcact

SEQ ID NO: 39 - DNA, 1158 bp, Homo sapiens
gcctgctgctctggcccctggtcctgtcctcttctccagcatggtgtgtctgaagctccctggaggctccagcttggcagcgttgacagtgacactgatggtgctgagctcccgactggctttcgctggggacacccgaccacgtttcttggagctgcgtaagtctgagtgtcatttcttcaatgggacggagcgggtgcggtacctggacagatacttccataaccaggaggagttcctgcgcttcgacagcgacgtgggggagtaccgggcggtgacggagctggggcggcctgtcgccgagtcctggaacagccagaaggacctcctggagcagaagcggggccgggtggacaattactgcagacacaactacggggttggtgagagcttcacagtgcagcggcgagtccatcctcaggtgactgtgtatcctgcaaagacccagcccctgcagcaccacaacctcctggtctgctctgtgagtggtttctatccaggcagcattgaagtcaggtggttccggaacggccaggaagagaaggctggggtggtgtccacgggcctgatccagaatggagactggaccttccagaccctggtgatgctagaaacagttcctcggagtggagaggtttacacttgccaagtggagcacccaagcgtaacgagcgctctcacagtggaatggagagcacggtctgaatctgcacagagcaagatgctgagtggagtcgggggctttgtgctgggcctgctcttccttggggccgggctgttcatctacttcaggaatcagaaaggacactctggacttcagccaacaggattcctgagctgaagtgcagatgacaatttaaggaagaatcttctgccccagctttgcaggatgaaaagctttcccgcctggctgttattcttccacgagagagggctttctcaggacctagttgctactggttcagcaactgcagaaaatgtcctcccttgtggcttcctcagttcctgcccttggcctgaagtcccagcattgatggcagcgcctcatcttcaacttttgtgctcccctttgcctaaaccctatggcctcctgtgcatctgtactcaccctgtaccacaaacacattacattattaaatgtttctcaaagatggagtt

SEQ ID NO: 40 - DNA, 2876 bp, Homo sapiens
tgaaacctaacccgccctggggaggcgcgcagcagaggctccgattcggggcaggtgagaggctgactttctctcggtgcgtccagtggagctctgagtttcgaatcggcggcggcggattccccgcgcgcccggcgtcggggcttccaggaggatgcggagccccagcgcggcgtggctgctgggggccgccatcctgctagcagcctctctctcctgcagtggcaccatccaaggaaccaatagatcctctaaaggaagaagccttattggtaaggttgatggcacatcccacgtcactggaaaaggagttacagttgaaacagtcttttctgtggatgagttttctgcatctgtcctcactggaaaactgaccactgtcttccttccaattgtctacacaattgtgtttgtggtgggtttgccaagtaacggcatggccctgtgggtctttcttttccgaactaagaagaagcaccctgctgtgatttacatggccaatctggccttggctgacctcctctctgtcatctggttccccttgaagattgcctatcacatacatggcaacaactggatttatggggaagctctttgtaatgtgcttattggctttttctatggcaacatgtactgttccattctcttcatgacctgcctcagtgtgcagaggtattgggtcatcgtgaaccccatggggcactccaggaagaaggcaaacattgccattggcatctccctggcaatatggctgctgattctgctggtcaccatccctttgtatgtcgtgaagcagaccatcttcattcctgccctgaacatcacgacctgtcatgatgttttgcctgagcagctcttggtgggagacatgttcaattacttcctctctctggccattggggtctttctgttcccagccttcctcacagcctctgcctatgtgctgatgatcagaatgctgcgatcttctgccatggatgaaaactcagagaagaaaaggaagagggccatcaaactcattgtcactgtcctggccatgtacctgatctgcttcactcctagtaaccttctgcttgtggtgcattattttctgattaagagccagggccagagccatgtctatgccctgtacattgtagccctctgcctctctacccttaacagctgcatcgacccctttgtctattactttgtttcacatgatttcagggatcatgcaaagaacgctctcctttgccgaagtgtccgcactgtaaagcagatgcaagtatccctcacctcaaagaaacactccaggaaatccagctcttactcttcaagttcaaccactgttaagacctcctattgagttttccaggtcctcagatgggaattgcacagtaggatgtggaacctgtttaatgttatgaggacgtgtctgttatttcctaatcaaaaaggtctcaccacataccatgtggatgcagcacctctcaggattgctaggagctcccctgtttgcatgagaaaagtagtcccccaaattaacatcagtgtctgtttcagaatctctctactcagatgaccccagaaactgaaccaacagaagcagacttttcagaagatggtgaagacagaaacccagtaacttgcaaaaagtagacttggtgtgaagactcacttctcagctgaaattatatatatacacatatatatattttacatctgggatcatgatagacttgttagggcttcaaggccctcagagatgatcagtccaactgaacgaccttacaaatgaggaaaccaagataaatgagctgccagaatcaggtttccaatcaacagcagtgagttgggattggacagtagaatttcaatgtccagtgagtgaggttcttgtaccacttcatcaaaatcatggatcttggctgggtgcggtgcctcatgcctgtaatcctagcactttgggaggctgaggcaggcaatcacttgaggtcaggagttcgagaccagcctggccatcatggcgaaacctcatctctactaaaaatacaaaagttaaccaggtgtgtggtgcacgtttgtaatcccagttactcaggaggctgaggcacaagaattgagtatcactttaactcaggaggcagaggttgcagtgagccgagattgcaccactgcactccagcttgggtgataaaataaaataaaatagtcgtgaatcttgttcaaaatgcagattcctcagattcaataatgagagctcagactgggaacagggcccaggaatctgtgtggtacaaacctgcatggtgtttatgcacacagagatttgagaaccattgttctgaatgctgcttccatttgacaaagtgccgtgataatttttgaaaagagaagcaaacaatggtgtctcttttatgttcagcttataatgaaatctgtttgttgacttattaggactttgaattatttctttattaaccctctgagtttttgtatgtattattattaaagaaaaatgcaatcaggattttaaacatgtaaatacaaattttgtataacttttgatgacttcagtgaaattttcaggtagtctgagtaatagattgttttgccacttagaatagcatttgccacttagtattttaaaaaataattgttggagtatttattgtcagttttgttcacttgttatctaatacaaaattataaagccttcagagggtttggaccacatctctttggaaaatagtttgcaacatatttaagagatacttgatgccaaaatgactttatacaacgattgtatttgtgacttttaaaaataattattttattgtgtaattgatttataaataacaaaattttttttacaacttaaaaaaaaaaaaaaa

SEQ ID NO: 41 - DNA, 2230 bp, Homo sapiens
cttggagacaacatgtggttcttgacaactctgctcctttgggttccagttgatgggcaagtggacaccacaaaggcagtgatcactttgcagcctccatgggtcagcgtgttccaagaggaaaccgtaaccttgcattgtgaggtgctccatctgcctgggagcagctctacacagtggtttctcaatggcacagccactcagacctcgacccccagctacagaatcacctctgccagtgtcaatgacagtggtgaatacaggtgccagagaggtctctcagggcgaagtgaccccatacagctggaaatccacagaggctggctactactgcaggtctccagcagagtcttcacggaaggagaacctctggccttgaggtgtcatgcgtggaaggataagctggtgtacaatgtgctttactatcgaaatggcaaagcctttaagtttttccactggaattctaacctcaccattctgaaaaccaacataagtcacaatggcacctaccattgctcaggcatgggaaagcatcgctacacatcagcaggaatatctgtcactgtgaaagagctatttccagctccagtgctgaatgcatctgtgacatccccactcctggaggggaatctggtcaccctgagctgtgaaacaaagttgctcttgcagaggcctggtttgcagctttacttctccttctacatgggcagcaagaccctgcgaggcaggaacacatcctctgaataccaaatactaactgctagaagagaagactctgggttatactggtgcgaggctgccacagaggatggaaatgtccttaagcgcagccctgagttggagcttcaagtgcttggcctccagttaccaactcctgtctggtttcatgtccttttctatctggcagtgggaataatgtttttagtgaacactgttctctgggtgacaatacgtaaagaactgaaaagaaagaaaaagtgggatttagaaatctctttggattctggtcatgagaagaaggtaatttccagccttcaagaagacagacatttagaagaagagctgaaatgtcaggaacaaaaagaagaacagctgcaggaaggggtgcaccggaaggagccccagggggccacgtagcagcggctcagtgggtggccatcgatctggaccgtcccctgcccacttgctccccgtgagcactgcgtacaaacatccaaaagttcaacaacaccagaactgtgtgtctcatggtatgtaactcttaaagcaaataaatgaactgacttcaactgggatacatttggaaatgtggtcatcaaagatgacttgaaatgaggcctactctaaagaattcttgaaaaacttacaagtcaagcctagcctgataatcctattacatagtttgaaaaatagtattttatttctcagaacaaggtaaaaaggtgagtgggtgcatatgtacagaagattaagacagagaaacagacagaaagagacacacacacagccaggagtgggtagatttcagggagacaagagggaatagtatagacaataaggaaggaaatagtacttacaaatgactcctaagggactgtgagactgagagggctcacgcctctgtgttcaggatacttagttcatggcttttctctttgactttactaaaagagaatgtctccatacgcgttctaggcatacaagggggtaactcatgatgagaaatggatgtgttattcttgccctctcttttgaggctctctcataacccctctatttctagagacaacaaaaatgctgccagtcctaggcccctgccctgtaggaaggcagaatgtaactgttctgtttgtttaacgattaagtccaaatctccaagtgcggcactgcaaagagacgcttcaagtggggagaagcggcgataccatagagtccagatcttgcctccagagatttgctttaccttcctgattttctggttactaattagcttcaggatacgctgctctcatacttgggctgtagtttggagacaaaatattttcctgccactgtgtaacatagctgaggtaaaaactgaactatgtaaatgactctactaaaagtttagggaaaaaaaacaggaggagtatgacacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 42 - DNA, 1985 bp, Homo sapiens
actgtggctaagtgtggggaccagacaggactcgtggagacatccaggtgctgaagccttcagctactgtctcagttttttgaagtttagcaatggcgtctttctctgctgagaccaattcaactgacctactctcacagccatggaatgagcccccagtaattctctccatggtcattctcagccttacttttttactgggattgccaggcaatgggctggtgctgtgggtggctggcctgaagatgcagcggacagtgaacacaatttggttcctccacctcaccttggcggacctcctctgctgcctctccttgcccttctcgctggctcacttggctctccagggacagtggccctacggcaggttcctatgcaagctcatcccctccatcattgtcctcaacatgtttgccagtgtcttcctgcttactgccattagcctggatcgctgtcttgtggtattcaagccaatctggtgtcagaatcatcgcaatgtagggatggcctgctctatctgtggatgtatctgggtggtggcttttgtgatgtgcattcctgtgttcgtgtaccgggaaatcttcactacagacaaccataatagatgtggctacaaatttggtctctccagctcattagattatccagacttttatggagatccactagaaaacaggtctcttgaaaacattgttcagccgcctggagaaatgaatgataggttagatccttcctctttccaaacaaatgatcatccttggacagtccccactgtcttccaacctcaaacatttcaaagaccttctgcagattcactccctaggggttctgctaggttaacaagtcaaaatctgtattctaatgtatttaaacctgctgatgtggtctcacctaaaatccccagtgggtttcctattgaagatcacgaaaccagcccactggataactctgatgcttttctctctactcatttaaagctgttccctagcgcttctagcaattccttctacgagtctgagctaccacaaggtttccaggattattacaatttaggccaattcacagatgacgatcaagtgccaacacccctcgtggcaataacgatcactaggctagtggtgggtttcctgctgccctctgttatcatgatagcctgttacagcttcattgtcttccgaatgcaaaggggccgcttcgccaagtctcagagcaaaacctttcgagtggccgtggtggtggtggctgtctttcttgtctgctggactccataccacatttttggagtcctgtcattgcttactgacccagaaactcccttggggaaaactctgatgtcctgggatcatgtatgcattgctctagcatctgccaatagttgctttaatcccttcctttatgccctcttggggaaagattttaggaagaaagcaaggcagtccattcagggaattctggaggcagccttcagtgaggagctcacacgttccacccactgtccctcaaacaatgtcatttcagaaagaaatagtacaactgtgtgaaaatgtggagcagccaacaagcaggggctcttaggcaatcacatagtgaaagtttataagaggatgaagtgatatggtgagcagcggacttcaaaaactgtcaaagaatcaatccagcggttctcaaacggtacacagactattgacatcagcatcacctagaaacttgttagaaatgcaaattctcaagccgcatcccagacttgctgaatcggaatctctgggggttgggacccagcaagggcacttaacaaaccctcgtttctgattaatgctaaatgtaagaatcattgtaaacattagttctatttctatcccaaactaagctatgtgaaataagagaagctactttgtttttaaatgatgttgaatatttgtcgatatttccatcattaaatttttccttagcattgtaaaaaaaaaaaaaaaaaaaaaaaaaaaaaa

SEQ ID NO: 43 - DNA, 587 bp, Homo sapiens
taaatcttttctgcttactgaaaaggaagagtctgatgattagttactgatcctctttgcatttgtaaagctttggagatattgaatcatgttaccatttctgtttttttccaccctgttttcttccatatttactgaagctcagaagcagtattgggtctgcaactcatccgatgcaagtatttcatacacctactgtgataaaatgcaatacccaatttcaattaatgttaacccctgtatagaattgaaaggatccaaaggattattgcacattttctacattccaaggagagatttaaagcaattatatttcaatctctatataactgtcaacaccatgaatcttccaaagcgcaaagaagttatttgccgaggatctgatgacgattactctttttgcagagctctgaagggagagactgtgaatacaacaatatcattctccttcaagggaataaaattttctaagggaaaatacaaatgtgttgttgaagctatttctgggagcccagaagaaatgctcttttgcttggagtttgtcatcctacaccaacctaattcaaattagaataaattgagtattt

SEQ ID NO: 44 - DNA, 2175 bp, Homo sapiens
gagaaattggagaagataaaactggacactggggagaccacaacttcatgctgcgtgggatctcccagctacctgcagtggccaccatgtcttgggtcctgctgcctgtactttggctcattgttcaaactcaagcaatagccataaagcaaacacctgaattaacgctccatgaaatagtttgtcctaaaaaacttcacattttacacaaaagagagatcaagaacaaccagacagaaaagcatggcaaagaggaaaggtatgaacctgaagttcaatatcagatgatcttaaatggagaagaaatcattctctccctacaaaaaaccaagcacctcctggggccagactacactgaaacattgtactcacccagaggagaggaaattaccacgaaacctgagaacatggaacactgttactataaaggaaacatcctaaatgaaaagaattctgttgccagcatcagtacttgtgacgggttgagaggatacttcacacatcatcaccaaagataccagataaaacctctgaaaagcacagacgagaaagaacatgccgtctttacatctaaccaggaggaacaagacccagctaaccacacatgtggtgtgaagagcactgacgggaaacaaggcccaattcgaatctctagatcactcaaaagcccagagaaagaagactttcttcgggcacagaaatacattgatctctatttggtgctggataatgccttttataagaactataatgagaatctaactctgataagaagctttgtgtttgatgtgatgaacctactcaatgtgatatataacaccatagatgttcaagtggccttggtaggtatggaaatctggtctgatggggataagataaaggtggtgcccagcgcaagcaccacgtttgacaacttcctgagatggcacagttctaacctggggaaaaagatccacgaccatgctcagcttctcagcgggattagcttcaacaatcgacgtgtgggactggcagcttcaaattccttgtgttccccatcttcggttgctgttattgaggctaaaaaaaagaataatgtggctcttgtaggagtgatgtcacatgagctgggccatgtccttggtatgcctgatgttccattcaacaccaagtgtccctctggcagttgtgtgatgaatcagtatctgagttcaaaattcccaaaggatttcagtacatcttgccgtgcacattttgaaagataccttttatctcagaaaccaaagtgcctgctgcaagcacctattcctacaaatataatgacaacaccagtgtgtgggaaccaccttctagaagtgggagaagactgtgattgtggctctcctaaggagtgtaccaatctctgctgtgaagccctaacgtgtaaactgaagcctggaactgattgcggaggagatgctccaaaccataccacagagtgaatccaaaagtctgcttcactgagatgctaccttgccaggacaagaaccaagaactctaactgtcccaggaatcttgtgaattttcacccataatggtctttcacttgtcattctactttctatattgttatcagtccaggaaacaggtaaacagatgtaattagagacattggctctttgtttaggcctaatctttctttttacttttttttttcttttttctttttttttaaagatcatgaatttgtgacttagttctgccctttggagaacaaaagaaagcagtcttccatcaaatcaccttaaaatgcacggctaaactattcagagttaacactccagaattgttaaattacaagtactatgctttaatgcttctttcatcttactagtatggcctataaaaaaaataataccacttgatgggtgaaggctttggcaatagaaagaagaatagaattcaggttttatgttattcctctgtgttcacttcgccttgctcttgaaagtgcagtatttttctacatcatgtcgagaatgattcaatgtaaatatttttcattttatcatgtatatcctatacacacatctccttcatcatcatatatgaagtttattttgagaagtctacattgcttacattttaattgagccagcaaagaaggcttaatgatttattgaaccataatgtcaataaaaacacaacttttgaggc

SEQ ID NO: 45 - DNA, 1893 bp, Homo sapiens
attagacagcacactgctgactgttttcagttgtttctgtaacagcagaaagtgcactcactaggagtagtcagaattcaaaatgctcaagagaaagccatccaatgtttcagagaaggagaaacatcaaaaaccaaagcgaagcagcagttttgggaatttcgatcgttttcggaataattctttatcaaaaccagatgattcaactgaggcacatgaaggagatcccacaaatggaagtggagaacaaagtaaaacttcaaataatggaggcggtttgggtaaaaaaatgagagctatttcatggacaatgaagaaaaaagtgggtaaaaagtacatcaaagccctttctgaggaaaaggatgaggaagatggagagaatgcccacccatatagaaacagtgaccctgtgattgggacccacacagagaaggtgtccctcaaagccagtgactccatggatagtctctacagtggacagagctcatcaagtggcataacaagctgttcagatggtacaagtaaccgggacagctttcgactggatgacgatggcccctattcaggaccattctgtggccgtgccagagtgcatacggatttcacgccaagtccctatgacactgactccctcaaaatcaagaaaggagacatcatagacattatttgcaaaacaccaatggggatgtggacaggaatgttgaacaataaagtgggaaacttcaaattcatttatgtggatgtcatctcagaagaggaagcagcccccaagaaaataaaggcaaaccgaaggagtaacagcaaaaaatccaagactctgcaggagttcctagagaggattcatctgcaggaatacacctcaacacttttgctcaatggttatgagactctagaagatttaaaagatataaaagagagtcacctcattgaattaaatattgaaaacccagatgacagaagaaggttactatcagctgctgaaaacttccttgaagaagaaattattcaagagcaagaaaatgaacctgagcccctatccttgagctcagacatctccttaaataagtcacagttagatgactgcccaagggactctggttgctatatctcatcaggaaattcagataatggcaaagaggatctggagtctgaaaatctgtctgacatggtacataagattattatcacagagccaagtgactgaacacgcattcccaactatatatctacagatgcattccattttaactcttcttgagctaaaacgtcaaataggagaggaagataagataaatatttgtaaataaaacctaaagtttaaatgttttaatctgaataattgtacataaaattttgtatctctaacattccaaattactgtcaataaaatatatatttattattttaaatgctatgtgttaatatttcacttgcttgtattagaaaggcaaaatgtaagactttggtatgtgtgacatatgctttatttggctttattttacaagtacagtatctgcaaaaaacaaagtaaccttttttcatacctgccagttttgaatttatatatgttattgaacaaatagtaatagaggattcgctgttgaaacaagttgtccaagcaatgttatattcatttttatacttattgggaaagtgtgagttaatattggacacattttatcctgatccacagtggagttttagtaattatattttgttgatttcttcattttgttttctggtataaaagtagagataatgtgtagtcacttctgatttagtgaaaccaattgtaataattgtggaaatgttttgtctttaagtgtaaatattttaaaatttgacataccctaatgttaataataaaaagaactatttgcatattgcaaaaaa

SEQ ID NO: 46 - DNA, 1019 bp, Homo sapiens
ttcctttcaaatacacaccccaacccgccccggcatacacagaaatggggactgcgagcagaagcaacatcgctcgccatctgcaaaccaatctcattctattttgtgtcggtgctgtgggcgcctgtactctctctgtcacacaaccgtggtacctagaagtggactacactcatgaggccgtcaccataaagtgtaccttctccgcaaccggatgcccttctgagcaaccaacatgcctgtggtttcgctacggtgctcaccagcctgagaacctgtgcttggacgggtgcaaaagtgaggcagacaagttcacagtgagggaggccctcaaagaaaaccaagtttccctcactgtaaacagagtgacttcaaatgacagtgcaatttacatctgtggaatagcattccccagtgtgccggaagcgagagctaaacagacaggaggagggaccacactggtggtaagagaaattaagctgctcagcaaggaactgcggagcttcctgacagctcttgtatcactgctctctgtctatgtgaccggtgtgtgcgtggccttcatactcctctccaaatcaaaatccaaccctctaagaaagaaagaaataaaagaagactcacaaaagaagaagagtgctcggcgtatttttcaggaaattgctcaagaactataccataagagacatgtggaaacaaatcagcaatctgagaaagataacaacacttatgaaaacagaagagtactttccaactatgaaaggccatagaaacgttttaattttcaatgaagtcactgaaaatccaactccaggagctatggcagtgttaatgaacatatatcatcaggtcttaaaaaaaaaataaaggtaaactgaaaagacaactggctacaaagaaggatgtcagaatgtaaggaaactataactaatagtcattaccaaaatactaaaacccaacaaaatgcaactgaaaaataccttccaaatttgccaagaaaaaaaattctattyyaaact

SEQ ID NO: 47 - DNA, 1702 bp, Homo sapiens
agactcaacaagagctccagcaaagactttcactgtagcttgacttgacctgagattaactagggaatcttgagaataaagatgagctctgaaaattgtttcgtagcagagaacagctctttgcatccggagagtggacaagaaaatgatgccaccagtccccatttctcaacacgtcatgaagggtccttccaagttcctgtcctgtgtgctgtaatgaatgtggtcttcatcaccattttaatcatagctctcattgccttatcagtgggccaatacaattgtccaggccaatacacattctcaatgccatcagacagccatgtttcttcatgctctgaggactgggttggctaccagaggaaatgctactttatttctactgtgaagaggagctggacttcagcccaaaatgcttgttctgaacatggtgctactcttgctgtcattgattctgaaaaggacatgaactttctaaaacgatacgcaggtagagaggaacactgggttggactgaaaaaggaacctggtcacccatggaagtggtcaaatggcaaagaatttaacaactggttcaacgttacagggtctgacaagtgtgtttttctgaaaaacacagaggtcagcagcatggaatgtgagaagaatttatactggatatgtaacaaaccttacaaataataaggaaacatgttcacttattgactattatagaatggaactcaaggaaatctgtgtcagtggatgctgctctgtggtccgaagtcttccatagagactttgtgaaaaaaaattttatagtgtcttgggaattttcttccaaacagaactatggaaaaaaaggaagaaattccaggaaaatctgcactgtgggcttttattgccatgagctagaagcatcacaggttgaccaataaccatgcccaagaatgagaagaatgactatgcaacctttggatgcactttatattattttgaatccagaaataatgaaataactaggcgtggacttactatttattgctgaatgactaccaacagtgagagcccttcatgcatttgcactactggaaggagttagatgttggtactagatactgaatgtaaacaaaggaattatggctggtaacataggtttttagtctaattgaatcccttaaactcagggagcatttataaatggacaaatgcttatgaaactaagatttgtaatatttctctctttttagagaaatttgccaatttactttgttatttttccccaaaaagaatgggatgatcgtgtatttatttttttacttcctcagctgtagacaggtccttttcgatggtacatatttctttgcctttataatcttttatacagtgtcttacagagaaaagacataagcaaagactatgaggaatatttgcaagacatagaatagtgttggaaaatgtgcaatatgtgatgtggcaaatctctattaggaaatattctgtaatcttcagacctagaataatactagtcttataataggtttgtgactttcctaaatcaattctattacgtgcaatacttcaatacttcatttaaaatatttttatgtgcaataaaatgtatttgtttgtattttgtgttcagtacaattataagctgtttttatatatgtgaaataaaagtagaataaacacaaaaaaaaaaaaaaaaaaaaaaaaaaaa

SEQ ID NO: 48 - DNA, 3806 bp, Homo sapiens
gaattcttagttgttttctttagaagaacatttctagggaataatacaagaagatttaggaatcattgaagttataaatctttggaatgagcaaactcagaatggtgctacttgaagactctggatctgctgacttcagaagacattttgtcaacttgagtcccttcaccattactgtggtcttacttctcagtgcctgttttgtcaccagttctcttggaggaacagacaaggagctgaggctagtggatggtgaaaacaagtgtagcgggagagtggaagtgaaagtccaggaggagtggggaacggtgtgtaataatggctggagcatggaagcggtctctgtgatttgtaaccagctgggatgtccaactgctatcaaagcccctggatgggctaattccagtgcaggttctggacgcatttggatggatcatgtttcttgtcgtgggaatgagtcagctctttgggattgcaaacatgatggatggggaaagcatagtaactgtactcaccaacaagatgctggagtgacctgctcagatggatccaatttggaaatgaggctgacgcgtggagggaatatgtgttctggaagaatagagatcaaattccaaggacggtggggaacagtgtgtgatgataacttcaacatagatcatgcatctgtcatttgtagacaacttgaatgtggaagtgctgtcagtttctctggttcatctaattttggagaaggctctggaccaatctggtttgatgatcttatatgcaacggaaatgagtcagctctctggaactgcaaacatcaaggatggggaaagcataactgtgatcatgctgaggatgctggagtgatttgctcaaagggagcagatctgagcctgagactggtagatggagtcactgaatgttcaggaagattagaagtgagattccaaggagaatgggggacaatatgtgatgacggctgggacagttacgatgctgctgtggcatgcaagcaactgggatgtccaactgccgtcacagccattggtcgagttaacgccagtaagggatttggacacatctggcttgacagcgtttcttgccagggacatgaacctgctgtctggcaatgtaaacaccatgaatggggaaagcattattgcaatcacaatgaagatgctggcgtgacatgttctgatggatcagatctggagctaagacttagaggtggaggcagccgctgtgctgggacagttgaggtggagattcagagactgttagggaaggtgtgtgacagaggctggggactgaaagaagctgatgtggtttgcaggcagctgggatgtggatctgcactcaaaacatcttatcaagtgtactccaaaatccaggcaacaaacacatggctgtttctaagtagctgtaacggaaatgaaacttctctttgggactgcaagaactggcaatggggtggacttacctgtgatcactatgaagaagccaaaattacctgctcagcccacagggaacccagactggttggaggggacattccctgttctggacgtgttgaagtgaagcatggtgacacgtggggctccatctgtgattcggacttctctctggaagctgccagcgttctatgcagggaattacagtgtggcacagttgtctctatcctggggggagctcactttggagagggaaatggacagatctgggctgaagaattccagtgtgagggacatgagtcccatctttcactctgcccagtagcaccccgcccagaaggaacttgtagccacagcagggatgttggagtagtctgctcaagatacacagaaattcgcttggtgaatggcaagaccccgtgtgagggcagagtggagctcaaaacgcttggtgcctggggatccctctgtaactctcactgggacatagaagatgcccatgttctttgccagcagcttaaatgtggagttgccctttctaccccaggaggagcacgttttggaaaaggaaatggtcagatctggaggcatatgtttcactgcactgggactgagcagcacatgggagattgtcctgtaactgctctaggtgcttcattatgtccttcagagcaagtggcctctgtaatctgctcaggaaaccagtcccaaacactgtcctcgtgcaattcatcgtctttgggcccaacaaggcctaccattccagaagaaagtgctgtggcctgcatagagagtggtcaacttcgcctggtaaatggaggaggtcgctgtgctgggagagtagagatctatcatgagggctcctggggcaccatctgtgatgacagctgggacctgagtgatgcccacgtggtttgcagacagctgggctgtggagaggccattaatgccactggttctgctcattttggggaaggaacagggcccatctggctggatgagatgaaatgcaatggaaaagaatcccgcatttggcagtgccattcacacggctgggggcagcaaaattgcaggcacaaggaggatgcgggagttatctgctcagaattcatgtctctgagactgaccagtgaagccagcagagaggcctgtgcagggcgtctggaagttttttacaatggagcttggggcactgttggcaagagtagcatgtctgaaaccactgtgggtgtggtgtgcaggcagctgggctgtgcagacaaagggaaaatcaaccctgcatctttagacaaggccatgtccattcccatgtgggtggacaatgttcagtgtccaaaaggacctgacacgctgtggcagtgcccatcatctccatgggagaagagactggccagcccctcggaggagacctggatcacatgtgacaacaagataagacttcaggaaggacccacttcctgttctggacgtgtggagatctggcatggaggttcctgggggacagtgtgtgatgactcttgggacttggacgatgctcaggtggtgtgtcaacaacttggctgtggtccagctttgaaagcattcaaagaagcagagtttggtcaggggactggaccgatatggctcaatgaagtgaagtgcaaagggaatgagtcttccttgtgggattgtcctgccagacgctggggccatagtgagtgtgggcacaaggaagacgctgcagtgaattgcacagatatttcagtgcagaaaaccccacaaaaagccacaacaggtcgctcatcccgtcagtcatcctttattgcagtcgggatccttggggttgttctgttggccattttcgtcgcattattcttcttgactaaaaagcgaagacagagacagcggcttgcagtttcctcaagaggagagaacttagtccaccaaattcaataccgggagatgaattcttgcctgaatgcagatgatctggacctaatgaattcctcagaaaattcccatgagtcagctgatttcagtgctgctgaactaatttctgtgtctaaatttcttcctatttctggaatggaaaaggaggccattctgagccacactgaaaaggaaaatgggaatttataacccagtgagttcagcctttaagataccttgatgaagacctggactattgaatggagcagaaattcacctctctcactgactattacagttgcatttttatggagttcttcttctcctaggattcctaagactgctgctgaatttataaaaattaagtttgtgaatgtgactacttagtggtgtatatgagactttcaagggaattaaataaataaataagaatgttattgatttgagtttgctttaatt

SEQ ID NO: 49 - DNA, 1315 bp, Homo sapiens
gcagttctttgaatttctcaccctaagatctggcctgtacattttcaaggaattcttgagaggttcttggagagattctgggagccaaacactccattgggatcctagctgttttagagaacaacttgtaatggagccttcatctcttgagctgccggctgacacagtgcagcgcattgcggctgaactcaaatgccacccaacggatgagagggtggctctccacctagatgaggaagataagctgaggcacttcagggagtgcttttatattcccaaaatacaggatctgcctccagttgatttatcattagtgaataaagatgaaaatgccatctatttcttgggaaattctcttggccttcaaccaaaaatggttaaaacatatcttgaagaagaactagataagtgggccaaaatagcagcctatggtcatgaagtggggaagcgtccttggattacaggagatgagagtattgtaggccttatgaaggacattgtaggagccaatgagaaagaaatagccctaatgaatgctttgactgtaaatttacatcttctaatgttatcattttttaagcctacgccaaaacgatataaaattcttctagaagccaaagccttcccttctgatcattatgctattgagtcacaactacaacttcacggacttaacattgaagaaagtatgcggatgataaagccaagagagggggaagaaaccttaagaatagaggatatccttgaagtaattgagaaggaaggagactcaattgcagtgatcctgttcagtggggtgcatttttacactggacagcactttaatattcctgccatcacaaaagctggacaagcgaagggttgttatgttggctttgatctagcacatgcagttggaaatgttgaactctacttacatgactggggagttgattttgcctgctggtgttcctacaagtatttaaatgcaggagcaggaggaattgctggtgccttcattcatgaaaagcatgcccatacgattaaacctgcgagatcggagttctttaattaggaatggaatgcaacagatttggacaagtcaaggacaagagctttagagagaccaaagagtttttcactgttaaagtgtccagtatgtagccgagaaccatatggagaacatcaaatacagtggaacaaatgtaactgctattgatgtcacactttgtgaagtagtctttgttgcttaaaaagggtgacatctagtggctaaacatgttatttcaaataaataatatcgaaataaaaaaaaaaaaaaaaaaaaaaaaaaaaa

SEQ ID NO: 50 - DNA, 1019 bp, Homo sapiens
cgaggctgcaccagcgcctggcaccatgaggacgcctgggcctctgcccgtgctgctgctgctcctggcgggagcccccgccgcgcggcccactcccccgacctgctactcccgcatgcgggccctgagccaggagatcacccgcgacttcaacctcctgcaggtctcggagccctcggagccatgtgtgagatacctgcccaggctgtacctggacatacacaattactgtgtgctggacaagctgcgggactttgtggcctcgcccccgtgttggaaagtggcccaggtagattccttgaaggacaaagcacggaagctgtacaccatcatgaactcgttctgcaggagagatttggtattcctgttggatgactgcaatgccttggaatacccaatcccagtgactacggtcctgccagatcgtcagcgctaagggaactgagaccagagaaagaacccaagagaactaaagttatgtcagctacccagacttaatgggccagagccatgaccctcacaggtcttgtgttagttgtatctgaaactgttatgtatctctctaccttctggaaaacagggctggtattcctacccaggaacctcctttgagcatagagttagcaaccatgcttctcattcccttgactcatgtcttgccaggatggttagatacacagcatgttgatttggtcactaaaaagaagaaaaggactaacaagcttcacttttatgaacaactattttgagaacatgcacaatagtatgtttttattactggtttaatggagtaatggtacttttattctttcttgatagaaacctgcttacatttaaccaagcttctattatgcctttttctaacacagactttcttcactgtctttcatttaaaaagaaattaatgctcttaagatatatattttacgtagtgctgacaggacccactctttcattgaaaggtgatgaaaatcaaataaagaatctcttcacatgagaaaaaaaaaaaaaaaaaaaaa

SEQ ID NO: 51 - DNA, 2545 bp, Homo sapiens
atccaatacaggagtgacttggaactccattctatcactatgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacataagagaccacttcaccaataagtattctgtgttaaaaatgttctattttaattataccgctatcattccaaaggaggatggcatataatacaaaggcttattaatttgactagaaaatttaaaacattactctgaaattgtaactaaagttagaaagttgattttaagaatccaaacgttaagaattgttaaaggctatgattgtctttgttcttctaccacccaccagttgaatttcatcatgcttaaggccatgattttagcaatacccatgtctacacagatgttcacccaaccacatcccactcacaacagctgcctggaagagcagccctaggcttccacgtactgcagcctccagagagtatctgaggcacatgtcagcaagtcctaagcctgttagcatgctggtgagccaagcagtttgaaattgagctggacctcaccaagctgctgtggccatcaacctctgtatttgaatcagcctacaggcctcacacacaatgtgtctgagagattcatgctgattgttattgggtatcaccactggagatcaccagtgtgtggctttcagagcctcctttctggctttggaagccatgtgattccatcttgcccgctcaggctgaccactttatttctttttgttcccctttgcttcattcaagtcagctcttctccatcctaccacaatgcagtgcctttcttctctccagtgcacctgtcatatgctctgatttatctgagtcaactcctttctcatcttgtccccaacaccccacagaagtgctttcttctcccaattcatcctcactcagtccagcttagttcaagtcctgcctcttaaataaacctttttggacacacaaattatcttaaaactcctgtttcacttggttcagtaccacatgggtgaacactcaatggttaactaattcttgggtgtttatcctatctctccaaccagattgtcagctccttgagggcaagagccacagtatatttccctgtttcttccacagtgcctaataatactgtggaactaggttttaataattttttaattgatgttgttatgggcaggatggcaaccagaccattgtctcagagcaggtgctggctctttcctggctactccatgttggctagcctctggtaacctcttacttattatcttcaggacactcactacagggaccagggatgatgcaacatccttgtctttttatgacaggatgtttgctcagcttctccaacaataagaagcacgtggtaaaacacttgcggatattctggactgtttttaaaaaatatacagtttaccgaaaatcatataatcttacaatgaaaaggactttatagatcagccagtgaccaaccttttcccaaccatacaaaaattccttttcccgaaggaaaagggctttctcaataagcctcagctttctaagatctaacaagatagccaccgagatccttatcgaaactcattttaggcaaatatgagttttattgtccgtttacttgtttcagagtttgtattgtgattatcaattaccacaccatctcccatgaagaaagggaacggtgaagtactaagcgctagaggaagcagccaagtcggttagtggaagcatgattggtgcccagttagcctctgcaggatgtggaaacctccttccaggggaggttcagtgaattgtgtaggagaggttgtctgtggccagaatttaaacctatactcactttcccaaattgaatcactgctcacactgctgatgatttagagtgctgtccggtggagatcccacccgaacgtcttatctaatcatgaaactccctagttccttcatgtaacttccctgaaaaatctaagtgtttcataaatttgagagtctgtgacccacttaccttgcatctcacaggtagacagtatataactaacaaccaaagactacatattgtcactgacacacacgttataatcatttatcatatatatacatacatgcatacactctcaaagcaaataatttttcacttcaaaacagtattgacttgtataccttgtaatttgaaatattttctttgttaaaatagaatggtatcaataaatagaccattaatcag

SEQ ID NO: 52 - DNA, 2201 bp, Homo sapiens
atcctgttgtgcctatcacctgttgaagttgccagtcttaaggaagggatcaatttctttcgcaataagagcactggcaaagactacgtcttgtacaagaataagagccgactgagggcatgcaagaatatgtgcaagcaccaaggaggcctgttcataaaagatatcgaggatttagccggaagttgttgaaatggatgaaaacaacggacttttgcttttagaactgaatcctcctaacccttgggacttacagcccagatctcctgaagagttggcttttggagaagtacagataacatatctcactcatgcctgcatggacctcaagttaggagacaagagaatggtgtttgacccttggttaatcggtcctgcttttgcccgtggatggtggttgctccatgagcctccatctgattggctggagaggctgtgccaggcagacctcgtttacatcagtcatctgcactcagaccacctgagttaccccacactgaaaaagcttgctgggagaagaccagatattcccatttatgttggaaacacagaaaggcctgtattttggaatctgaatcagagcggtgtccagttgactaatatcaatgtcgtgccatttggaatatggcagcaggtggacaaaaatcttcgattcatgatcttgatggatggtgttcatcctgagatggacacttgcattattgtggagtacaaaggtcataaaatactcaatacagtagactgcaccagacccaatgggggaaggctgcctatgaaggttgctctaatgatgagtgattttgctggaggagcatcaggctttccaatgactttcagtggtggaaaatttacggaggaatggaaagcccaattcattaaaacagaaaggaagaagctcctgaactacaaggcttggctggtgaagaacctgcaaccccgaatttattgtccctttgctgggtattttgtggaatctcacccatcagacaagtacatcaaggaaacaaacaccaaaaatgacccaaatgaactcaacaatcttatcaagaaaaactctgatgtgataacatggacccctcgaccgggagccacccttgatctgggaagaatgctgaaggatccaacagacagcaagggcatcatagagcctccagaggggacaaaaatttacaaggattcctgggattttgaaccttatttggaaatcttgaatgctgctctaggagatgaaatatttcttcactcatcctggataaaagaatacttcacttgggctggatttaaagattacaaccttgtggtcaggatgattgagacagatgaggacttcaatccttttcctggaggatatgactatttggttgactttttagatttatccttcccaaaagaaagaccacaacgagaacatccctatgaggaaatccatagccgggtggatgtcatcagacacgtggtgaagaatggtctactctgggatgagttgtatataggattccaaacacggctccagcgggatcctgacatataccatcacctgttttggaatcattttcaaataaaactccccctcacaccacccaactggaagtcattcctgatgtgctgtgagcagaatgggcctgcgattttgcaagaatgcaaaaccacatgaaaatttcaagaattcactgatctgatgcaaaataaaaatttatcattacatcttgaacccaggaagcttacagcaaagagactatgctttatgacgtcagcaatagataattccacgttgcctttgtgatttgtatatatagcttacatttgtggaccactacatagccagattcaaaaatattttacttgttccatccacagttctctacagaaagaaccaatgaacccaataggaacaaattctctgtggaaaacaaagcatagctgtagtagatacgaatccaatcacagaggaaacaggaagagaaaaacatccaagactacagtgaaaactggaaatggtctgttttcgtgatattcgtatgattaagatgcaaattttttcttaggaaaatgtgattgttaactagcattctgttttacatgttgacatttctaacacacacaccactgatttgaacttcaaaatttattttctgattatatatgctaggtctgattctgaagatacaagaattcaatggtggaatttgtctcctgaaatt

SEQ ID NO: 53 - DNA, 1787 bp, Homo sapiens
ttcttgacttgatgcaggcacagatttatcaagctcctcagtcaacaaacacatcaccggaagaaacatggaaggaaaggaattttaaaaggaaataccaatctctgtgcaaacaaagccttgtatattcatgtttgcaccaatctactgtgagatttatgaagaaaaacaaattgcggacaactctctatgtacacttacaaatgcctcagttgatgcttgtgggctgtttgtcagcgttctgtgataatgaacacatggacttctgtttattaaattcagttgacccctttagccaattgccaggagcctggatttttacttccaactgctgatatctgtgtaaaaattgatctacatccaccctttaaaagcattgatgaattaattagaactttagacaacaaagaaaaattgaaaaagaattctcagtaaaagcgaattcgatgttcaaaacaaactacaaagagacaagacttctctgtttactttctaagaactaatataattgctaccttaaaaaggaaaaaatgaacagcacatgtattgaagaacagcatgacctggatcactatttgtttcccattgtttacatctttgtgattatagtcagcattccagccaatattggatctctgtgtgtgtctttcctgcaagcaaagaaggaaagtgaactaggaatttacctcttcagtttgtcactatcagatttactctatgcattaactctccctttatggattgattatacttggaataaagacaactggactttctctcctgccttgtgcaaagggagtgcttttctcatgtacatgaatttttacagcagcacagcattcctcacctgcattgccgttgatcggtatttggctgttgtctaccctttgaagttttttttcctaaggacaagaagatttgcactcatggtcagcctgtccatctggatattggaaaccatcttcaatgctgtcatgttgtgggaagatgaaacagttgttgaatattgcgatgccgaaaagtctaattttactttatgctatgacaaataccctttagagaaatggcaaatcaacctcaacttgttcaggacgtgtacaggctatgcaatacctttggtcaccatcctgatctgcaaccggaaagtctaccaagctgtgcggcacaataaagccacggaaaacaaggaaaagaagagaatcataaaactacttgtcagcatcacagttacttttgtcttatgctttactccctttcatgtgatgttgctgattcgctgcattttagagcatgctgtgaacttcgaagaccacagcaattctgggaagcgaacttacacaatgtatagaatcacggttgcattaacaagtttaaattgtgttgctgatccaattctgtactgttttgtaaccgaaacaggaagatatgatatgtggaatatattaaaattctgcactgggaggtgtaatacatcacaaagacaaagaaaacgcatactttctgtgtctacaaaagatactatggaattagaggtccttgagtagaaccaaggatgttttgaagggaagggaagtttaagttatgcattattatatcatcaagattacattttgaaaaggaaatctagcatgtgaggggactaagtgttctcagagtgatgttttaatccagtccaataaaaatatcttaaaactgcattgtacagctccctccctgcgttttattaaatgatgtatattaaacaaagatcaataaaaaaaaaaaaaaaaaaaaaaaaaaaaaaaaaa

SEQ ID NO: 54 - DNA, 1908 bp, Homo sapiens
catttattaaggactctctgctccagcctctcactctcactctcctccgctcaaactcagctcacttgagagtctcctcccgccagctgtggaaagaactttgcgtctctccagcaatgcatctccttgcgattctgttttgtgctctctggtctgcagtgttggccgagaactcggatgattatgatctcatgtatgtgaatttggacaacgaaatagacaatggactccatcccactgaggaccccacgccgtgcgactgcggtcaggagcactcggaatgggacaagctcttcatcatgctggagaactcgcagatgagagagcgcatgctgctgcaagccacggacgacgtcctgcggggcgagctgcagaggctgcgggaggagctgggccggctcgcggaaagcctggcgaggccgtgcgcgccgggggctcccgcagaggccaggctgaccagtgctctggacgagctgctgcaggcgacccgcgacgcgggccgcaggctggcgcgtatggagggcgcggaggcgcagcgcccagaggaggcggggcgcgccctggccgcggtgctagaggagctgcggcagacgcgagccgacctgcacgcggtgcagggctgggctgcccggagctggctgccggcaggttgtgaaacagctattttattcccaatgcgttccaagaagatttttggaagcgtgcatccagtgagaccaatgaggcttgagtcttttagtgcctgcatttgggtcaaagccacagatgtattaaacaaaaccatcctgttttcctatggcacaaagaggaatccatatgaaatccagctgtatctcagctaccaatccatagtgtttgtggtgggtggagaggagaacaaactggttgctgaagccatggtttccctgggaaggtggacccacctgtgcggcacctggaattcagaggaagggctcacatccttgtgggtaaatggtgaactggcggctaccactgttgagatggccacaggtcacattgttcctgagggaggaatcctgcagattggccaagaaaagaatggctgctgtgtgggtggtggctttgatgaaacattagccttctctgggagactcacaggcttcaatatctgggatagtgttcttagcaatgaagagataagagagaccggaggagcagagtcttgtcacatccgggggaatattgttgggtggggagtcacagagatccagccacatggaggagctcagtatgtttcataaatgttgtgaaactccacttgaagccaaagaaagaaactcacacttaaaacacatgccagttgggaaggtctgaaaactcagtgcataataggaacacttgagactaatgaaagagagagttgagaccaatctttatttgtactggccaaatactgaataaacagttgaaggaaagacattggaaaaagcttttgaggataatgttactagactttatgccatggtgctttcagtttaatgctgtgtctctgtcagataaactctcaaataattaaaaaggactgtattgttgaacagagggacaattgttttacttttctttggttaattttgttttggccagagatgaattttacattggaagaataacaaaataagatttgttgtccattgttcattgttattggtatgtaccttattacaaaaaaaatgatgaaaacatatttatactacaaggtgacttaacaactataaatgtagtttatgtgttataatcgaatgtcacgtttttgagaagatagtcatataagttatattgcaaaagggatttgtattaatttaagactatttttgtaaagctctactgtaaataaaatattttataaaactaaaaaaaaaaaaaaaaaaaaaaaaaaaaaaaaaaaaa

SEQ ID NO: 55 - DNA, 2116 bp, Homo sapiens
acatccgcggcaacgcctccttggtgtcgtccgcttccaataacccagcttgcgtcctgcacacttgtggcttccgtgcacacattaacaactcatggttctagctcccagtcgccaagcgttgccaaggcgttgagagatcatctgggaagtcttttacccagaattgctttgattcaggccagctggtttttcctgcggtgattcggaaattcgcgaattcctctggtcctcatccaggtgcgcgggaagcaggtgcccaggagagaggggataatgaagattccatgctgatgatcccaaagattgaacctgcagaccaagcgcaaagtagaaactgaaagtacactgctggcggatcctacggaagttatggaaaaggcaaagcgcagagccacgccgtagtgtgtgccgccccccttgggatggatgaaactgcagtcgcggcgtgggtaagaggaaccagctgcagagatcaccctgcccaacacagactcggcaactccgcggaagaccagggtcctgggagtgactatgggcggtgagagcttgctcctgctccagttgcggtcatcatgactacgcccgcctcccgcagaccatgttccatgtttcttttaggtatatctttggacttcctcccctgatccttgttctgttgccagtagcatcatctgattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacactgaaaaatatggagtggcaatatagaaacacgaactttagctgcatcctccaagaatctatctgcttatgcagtttttcagagtggaatgcttcctagaagttactgaatgcaccatggtcaaaacggattagggcatttgagaaatgcatattgtattactagaagatgaatacaaacaatggaaactgaatgctccagtcaacaaactatttcttatatatgtgaacatttatcaatcagtataattctgtactgatttttgtaagacaatccatgtaaggtatcagttgcaataatacttctcaaacctgtttaaatatttcaagacattaaatctatgaagtatataatggtttcaaagattcaaaattgacattgctttactgtcaaaataattttatggctcactatgaatctattatactgtattaagagtgaaaattgtcttcttctgtgctggagatgttttagagttaacaatgatatatggataatgccggtgagaataagagagtcataaaccttaagtaagcaacagcataacaaggtccaagatacctaaaagagatttcaagagatttaattaatcatgaatgtgtaacacagtgccttcaataaatggtatagcaaatgttttgacatgaaaaaaggacaatttcaaaaaaataaaataaaataaaaataaattcacctagtctaaggatgctaaaccttagtactgagttacattgtcatttatatagattataacttgtctaaataagtttgcaatttgggagatatatttttaagataataatatatgtttaccttttaattaatgaaatatctgtatttaattttgacactatatctgtatataaaatattttcatacagcattacaaattgcttactttggaatacatttctcctttgataaaataaatgagctatgtattaacaaaaaaaaaaaaaaaaaaaaaaaaaaaaaaaaaaaaaaa

SEQ ID NO: 56 - DNA, 3197 bp, Homo sapiens
ggaccacagctcctcccgtgcatccactcggcctgggaggttctggattttggctgtcgagggagtttgcctgcctctccagagaaagatggtcatgaggcccctgtggagtctgcttctctgggaagccctacttcccattacagttactggtgcccaagtgctgagcaaagtcgggggctcggtgctgctggtggcagcgcgtccccctggcttccaagtccgtgaggctatctggcgatctctctggccttcagaagagctcctggccacgtttttccgaggctccctggagactctgtaccattcccgcttcctgggccgagcccagctacacagcaacctcagcctggagctcgggccgctggagtctggagacagcggcaacttctccgtgttgatggtggacacaaggggccagccctggacccagaccctccagctcaaggtgtacgatgcagtgcccaggcccgtggtacaagtgttcattgctgtagaaagggatgctcagccctccaagacctgccaggttttcttgtcctgttgggcccccaacatcagcgaaataacctatagctggcgacgggagacaaccatggactttggtatggaaccacacagcctcttcacagacggacaggtgctgagcatttccctgggaccaggagacagagatgtggcctattcctgcattgtctccaaccctgtcagctgggacttggccacagtcacgccctgggatagctgtcatcatgaggcagcaccagggaaggcctcctacaaagatgtgctgctggtggtggtgcctgtctcgctgctcctgatgctggttactctcttctctgcctggcactggtgcccctgctcagggaaaaagaaaaaggatgtccatgctgacagagtgggtccagagacagagaacccccttgtgcaggatctgccataaaggacaatatgaactgatgcctggactatcagtaaccccactgcacaggcacacgatgctctgggacataactggtgcctggaaatcaccatggtcctcatatctcccatgggaatcctgtcctgcctcgaaggagcagcctgggcagccatcacaccacgaggacaggaagcaccagcacgtttcacacctcccccttccctctcccatcttctcatatcctggctcttctctgggcaagatgagccaagcagaacattccatccaggacactggaagttctccaggatccagatccatggggacattaatagtccaaggcattccctcccccaccactattcataaagtattaaccaactggcaccaaggaattgcctccagcctgagtcctaggctctaaaagatattacatatttgaactaatagaggaactctgagtcacccatgccagcatcagcttcagccccagaccctgcagtttgagatctgatgcttcctgagggccaaggcattgctgtaagaaaaggtctagaaataggtgaaagtgagaggtgggggacaggggtttctctttctggcctaaggactttcaggtaatcagagttcatgggccctcaaaggtaaattgcagttgtagacaccgaggatggttgacaacccatggttgagatgggcaccgttttgcaggaaacaccatattaatagacatcctcaccatctccatccgctctcacgcctcctgcaggatctgggagtgagggtggagagtctttcctcacgctccagcacagtggccaggaaaagaaatactgaatttgccccagccaacaggacgttcttgcacaacttcaagaaaagcagctcagctcaggatgagtcttcctgcctgaaactgagagagtgaagaaccataaaacgctatgcagaaggaacattatggagagaaagggtactgaggcactctagaatctgccacattcattttcaaatgcaaatgcagaagacttaccttagttcaaggggaggggacaaagaccccacagcccaacagcaggactgtagaggtcactctgactccatcaaactttttattgtggccatcttaggaaaatacattctgcccctgaatgattctgtctagaaaagctctggagtattgatcactactggaaaaacacttaaggagctaaacttaccttcggggattattagctgataaggttcacagtttctctcacccaggtgtaactggattttttctggggcctcaatccagtcttgataacagcgaggaaagaggtattgaagaaacaggggtgggtttgaagtactattttccccagggtggcttcaatctccccacctaggatgtcagccctgtccaaggaccttccctcttctcccccagttccctgggcaatcacttcaccttggacaaaggatcagcacagctggcctccagatccacatcaccactcttccactcgattgttcccagatcctccctgcctggcctgctcagaggttccctgttggtaacctggctttatcaaattctcatccctttcccacacccacttctctcctatcaccttcccccaagattacctgaacagggtccatggccactcaacctgtcagcttgcaccatccccacctgccacctacagtcaggccacatgcctggtcactgaatcatgcaaaactggcctcagtccctaaaaatgatgtggaaaggaaagcccaggatctgacaatgagccctggtggatttgtggggaaaaaatacacagcactccccacctttctttcgttcatctccagggccccacctcagatcaaagcagctctggatgagatgggacctgcagctctccctccacaaggtgactcttagcaacctcatttcgacagtggtttgtagcgtggtgcaccagggccttgttgaacagatccacactgctctaataaagttcccatccttaatgactcacttgtcaactagtggactaattaaccctccaccaaaaaaacacaaagtgcttctgtgagaccaattttgtgctaatgagcattgagactgatgctttgtaagtcacaccacaacaaatattgattgagggcgctgcatgtgctgggtacatttcttggcacttgggaatcagtagtcaagcgaaacccttgcctttgagagtttatggtctggataatataaataaacaagtaagcataaaaaaaaaaaaaaaaa

SEQ ID NO: 57 - DNA, 1959 bp, Homo sapiens
tctcacggagacggaccacagcaagcagaggctgggggggggaaagacgaggaaagaggaggaaaacaaaagctgctacttatggaagatacaaaggagtctaacgtgaagacattttgctccaagaatatcctagccatccttggcttctcctctatcatagctgtgatagctttgcttgctgtggggttgacccagaacaaagcattgccagaaaacgttaagtatgggattgtgctggatgcgggttcttctcacacaagtttatacatctataagtggccagcagaaaaggagaatgacacaggcgtggtgcatcaagtagaagaatgcagggttaaaggtcctggaatctcaaaatttgttcagaaagtaaatgaaataggcatttacctgactgattgcatggaaagagctagggaagtgattccaaggtcccagcaccaagagacacccgtttacctgggagccacggcaggcatgcggttgctcaggatggaaagtgaagagttggcagacagggttctggatgtggtggagaggagcctcagcaactacccctttgacttccagggtgccaggatcattactggccaagaggaaggtgcctatggctggattactatcaactatctgctgggcaaattcagtcagaaaacaaggtggttcagcatagtcccatatgaaaccaataatcaggaaacctttggagctttggaccttgggggagcctctacacaagtcacttttgtaccccaaaaccagactatcgagtccccagataatgctctgcaatttcgcctctatggcaaggactacaatgtctacacacatagcttcttgtgctatgggaaggatcaggcactctggcagaaactggccaaggacattcaggttgcaagtaatgaaattctcagggacccatgctttcatcctggatataagaaggtagtgaacgtaagtgacctttacaagaccccctgcaccaagagatttgagatgactcttccattccagcagtttgaaatccagggtattggaaactatcaacaatgccatcaaagcatcctggagctcttcaacaccagttactgcccttactcccagtgtgccttcaatgggattttcttgccaccactccagggggattttggggcattttcagctttttactttgtgatgaagtttttaaacttgacatcagagaaagtctctcaggaaaaggtgactgagatgatgaaaaagttctgtgctcagccttgggaggagataaaaacatcttacgctggagtaaaggagaagtacctgagtgaatactgcttttctggtacctacattctctccctccttctgcaaggctatcatttcacagctgattcctgggagcacatccatttcattggcaagatccagggcagcgacgccggctggactttgggctacatgctgaacctgaccaacatgatcccagctgagcaaccattgtccacacctctctcccactccacctatgtcttcctcatggttctattctccctggtccttttcacagtggccatcataggcttgcttatctttcacaagccttcatatttctggaaagatatggtatagcaaaagcagctgaaatatgctggctggagtgaggaaaaaaatcgtccagggagcattttcctccatcgcagtgttcaaggccatccttccctgtctgccagggccagtcttgacgagtgtgaagcttccttggcttttactgaagcctttcttttggaggtattcaatatcctttgcctcaaggacttcggcagatactgtctctttcatgagtttttcccagctacacctttctcctttgtactttgtgcttgtataggttttaaagacctgacacctttcataatctttgctttataaaagaacaatattgactttgtctagaaaaaaaaaaaaaaaaaaaaa

SEQ ID NO: 58 - DNA, 2690 bp, Homo sapiens
ataaaaacccagaaagccccagaaacaaagacttcacggacaaagtcccttggaaccagagagaagccgggatggaaactccaaacaccacagaggactatgacacgaccacagagtttgactatggggatgcaactccgtgccagaaggtgaacgagagggcctttggggcccaactgctgccccctctgtactccttggtatttgtcattggcctggttggaaacatcctggtggtcctggtccttgtgcaatacaagaggctaaaaaacatgaccagcatctacctcctgaacctggccatttctgacctgctcttcctgttcacgcttcccttctggatcgactacaagttgaaggatgactgggtttttggtgatgccatgtgtaagatcctctctgggttttattacacaggcttgtacagcgagatctttttcatcatcctgctgacgattgacaggtacctggccatcgtccacgccgtgtttgccttgcgggcacggaccgtcacttttggtgtcatcaccagcatcatcatttgggccctggccatcttggcttccatgccaggcttatacttttccaagacccaatgggaattcactcaccacacctgcagccttcactttcctcacgaaagcctacgagagtggaagctgtttcaggctctgaaactgaacctctttgggctggtattgcctttgttggtcatgatcatctgctacacagggattataaagattctgctaagacgaccaaatgagaagaaatccaaagctgtccgtttgatttttgtcatcatgatcatcttttttctcttttggaccccctacaatttgactatacttatttctgttttccaagacttcctgttcacccatgagtgtgagcagagcagacatttggacctggctgtgcaagtgacggaggtgatcgcctacacgcactgctgtgtcaacccagtgatctacgccttcgttggtgagaggttccggaagtacctgcggcagttgttccacaggcgtgtggctgtgcacctggttaaatggctccccttcctctccgtggacaggctggagagggtcagctccacatctccctccacaggggagcatgaactctctgctgggttctgactcagaccataggaggccaacccaaaataagcaggcgtgacctgccaggcacactgagccagcagcctggctctcccagccaggttctgactcttggcacagcatggagtcacagccacttgggatagagagggaatgtaatggtggcctggggcttctgaggcttctggggcttcagtcttttccatgaacttctcccctggtagaaagaagatgaatgagcaaaaccaaatattccagagactgggactaagtgtaccagagaagggcttggactcaagcaagatttcagatttgtgaccattagcatttgtcaacaaagtcacccacttcccactattgcttgcacaaaccaattaaacccagtagtggtgactgtgggctccattcaaagtgagctcctaagccatgggagacactgatgtatgaggaatttctgttcttccatcacctccccccccccgccaccctcccactgccaaagaacttggaaatagtgatttccacagtgactccactctgagtcccagagccaatcagtagccagcatctgcctccccttcactcccaccgcaggatttgggctcttggaatcctggggaacatagaactcatgacggaagagttgagacctaacgagaaatagaaatggggaactactgctggcagtggaactaagaaagcccttaggaagaatttttatatccactaaaatcaaacaattcagggagtgggctaagcacgggccatatgaataacatggtgtgcttcttaaaatagccataaaggggagggactcatcatttccatttacccttcttttctgactatttttcagaatctctcttcttttcaagttgggtgatatgttggtagattctaatggctttattgcagcgattaataacaggcaaaaggaagcagggttggtttcccttctttttgttcttcatctaagccttctggttttatgggtcagagttccgactgccatcttggacttgtcagcaaaaaaaaaaaataataataataataaggcctgctgtgtaagctgacagtatttgtagctgatagggggttgggaggaaagtgtctactaggagggtggggtgagattctgtgttgatgtaggaggccgagaaggcccttaactcaaagtagcttatttatccaaaatgttctggatgcatcatctccaaccaaggaccccttatttatcatgcctttgttctcttttccctcagatgtatatttctttaaaaataattttcctaataacaaaacttatttctaaaacagcttaaaaattcaaagaaaaaccccaaacactgacattacctacacttccactacccaaagacaaaatgtgcccactgtgtgcttttgagtgtattttcttttagtttgttttttgttgggtgcatatttatgataataacaatgatggacttcaattgtactcactgttctattgttggttttaattagcagcaagttgtgatcactttcccaggtgaataaatcatttcaaagcattaaaaaaaaaaaaaa

SEQ ID NO: 59 - protein, 196 aa, Homo sapiens
MPGMFFSANPKELKGTTHSLLDDKMQKRRPKTFGMDMKAYLRSMIPHLESGMKSSKSKDVLSAAEVMQWSQSLEKLLANQTGQNVFGSFLKSEFSEENIEFWLACEDYKKTESDLLPCKAEEIYKAFVHSDAAKQINIDFRTRESTAKKIKAPTPTCFDEAQKVIYTLMEKDSYPRFLKSDIYLNLLNDLQANSLK

SEQ ID NO: 60 - protein, 543 aa, Homo sapiens
MLLRSKPALPPPLMLLLLGPLGPLSPGALPRPAQAQDVVDLDFFTQEPLHLVSPSFLSVTIDANLATDPRFLILLGSPKLRTLARGLSPAYLRFGGTKTDFLIFDPKKESTFEERSYWQSQVNQDICKYGSIPPDVEEKLRLEWPYQEQLLLREHYQKKFKNSTYSRSSVDVLYTFANCSGLDLIFGLNALLRTADLQWNSSNAQLLLDYCSSKGYNISWELGNEPNSFLKKADIFINGSQLGEDFIQLHKLLRKSTFKNAKLYGPDVGQPRRKTAKMLKSFLKAGGEVIDSVTWHHYYLNGRTATREDFLNPDVLDIFISSVQKVFQVVESTRPGKKVWLGETSSAYGGGAPLLSDTFAAGFMWLDKLGLSARMGIEVVMRQVFFGAGNYHLVDENFDPLPDYWLSLLFKKLVGTKVLMASVQGSKRRKLRVYLHCTNTDNPRYKEGDLTLYAINLHNVTKYLRLPYPFSNKQVDKYLLRPLGPHGLLSKSVQLNGLTLKMVDDQTLPPLMEKPLRPGSSLGLPAFSYSFFVIRNAKVAACI

SEQ ID NO: 61 - protein, 481 aa, Homo sapiens
MALSYRVSELQSTIPEHILQSTFVHVISSNWSGLQTESIPEEMKQIVEEQGNKLHWAALLILMVIIPTIGGNTLVILAVSLEKKLQYATNYFLMSLAVADLLVGLFVMPIALLTIMFEAMWPLPLVLCPAWLFLDVLFSTASIMHLCAISVDRYIAIKKPIQANQYNSRATAFIKITVVWLISIGIAIPVPIKGIETDVDNPNNITCVLTKERFGDFMLFGSLAAFFTPLAIMIVTYFLTIHALQKKAYLVKNKPPQRLTWLTVSTVFQRDETPCSSPEKVAMLDGSRKDKALPNSGDETLMRRTSTIGKKSVQTISNEQRASKVLGIVFFLFLLMWCPFFITNITLVLCDSCNQTTLQMLLEIFVWIGYVSSGVNPLVYTLFNKTFRDAFGRYITCNYRATKSVKTLRKRSSKIYFRNPMAENSKFFKKHGIRNGINPAMYQSPMRLRSSTIQSSSIILLDTLLLTENEGDKTEERVSYV

SEQ ID NO: 62 - protein, 416 aa, Homo sapiens
MPRQLSAAAALFASLAVILHDGSQMRAKAFPETRDYSQPTAAATVQDIKKPVQQPAKQAPHQTLAARFMDGHITFQTAATVKIPTTTPATTKNTATTSPITYTLVTTQATPNNSHTAPPVTEVTVGPSLAPYSLPPTITPPAHTTGTSSSTVSHTTGNTTQPSNQTTLPATLSIALHKSTTGQKPVQPTHAPGTTAAAHNTTRTAAPASTVPGPTLAPQPSSVKTGIYQVLNGSRLCIKAEMGIQLIVQDKESVFSPRRYFNIDPNATQASGNCGTRKSNLLLNFQGGFVNLTFTKDEESYYISEVGAYLTVSDPETIYQGIKHAVVMFQTAVGHSFKCVSEQSLQLSAHLQVKTTDVQLQAFDFEDDHFGNVDECSSDYTIVLPVIGAIVVGLCLMGMGVYKIRLRCQSSGYQRI

SEQ ID NO: 63 - protein, 61 aa, Homo sapiens
MKRFLFLLLTISLLVMVQIQTGLSGQNDTSQTSSPSASSNISGGIFLFFVANAIIHLFCFS

SEQ ID NO: 64 - protein, 1403 aa, Homo sapiens
MATQQKASDERISQFDHNLLPELSALLGLDAVQLAKELEEEEQKERAKMQKGYNSQMRSEAKRLKTFVTYEPYSSWIPQEMAAAGFYFTGVKSGIQCFCCSLILFGAGLTRLPIEDHKRFHPDCGFLLNKDVGNIAKYDIRVKNLKSRLRGGKMRYQEEEARLASFRNWPFYVQGISPCVLSEAGFVFTGKQDTVQCFSCGGCLGNWEEGDDPWKEHAKWFPKCEFLRSKKSSEEITQYIQSYKGFVDITGEHFVNSWVQRELPMASAYCNDSIFAYEELRLDSFKDWPRESAVGVAALAKAGLFYTGIKDIVQCFSCGGCLEKWQEGDDPLDDHTRCFPNCPFLQNMKSSAEVTPDLQSRGELCELLETTSESNLEDSIAVGPIVPEMAQGEAQWFQEAKNLNEQLRAAYTSASFRHMSLLDISSDLATDHLLGCDLSIASKHISKPVQEPLVLPEVFGNLNSVMCVEGEAGSGKTVLLKKIAFLWASGCCPLLNRFQLVFYLSLSSTRPDEGLASIICDQLLEKEGSVTEMCMRNIIQQLKNQVLFLLDDYKEICSIPQVIGKLIQKNHLSRTCLLIAVRTNRARDIRRYLETILEIKAFPFYNTVCILRKLFSHNMTRLRKFMVYFGKNQSLQKIQKTPLFVAAICAHWFQYPFDPSFDDVAVFKSYMERLSLRNKATAEILKATVSSCGELALKGFFSCCFEFNDDDLAEAGVDEDEDLTMCLMSKFTAQRLRPFYRFLSPAFQEFLAGMRLIELLDSDRQEHQDLGLYHLKQINSPMMTVSAYNNFLNYVSSLPSTKAGPKIVSHLLHLVDNKESLENISENDDYLKHQPEISLQMQLLRGLWQICPQAYFSMVSEHLLVLALKTAYQSNTVAACSPFVLQFLQGRTLTLGALNLQYFFDHPESLSLLRSIHFPIRGNKTSPRAHFSVLETCFDKSQVPTIDQDYASAFEPMNEWERNLAEKEDNVKSYMDMQRRASPDLSTGYWKLSPKQYKIPCLEVDVNDIDVVGQDMLEILMTVFSASQRIELHLNHSRGFIESIRPALELSKASVTKCSISKLELSAAEQELLLTLPSLESLEVSGTIQSQDQIFPNLDKFLCLKELSVDLEGNINVFSVIPEEFPNFHHMEKLLIQISAEYDPSKLVKLIQNSPNLHVFHLKCNFFSDFGSLMTMLVSCKKLTEIKFSDSFFQAVPFVASLPNFISLKILNLEGQQFPDEETSEKFAYILGSLSNLEELILPTGDGIYRVAKLIIQQCQQLHCLRVLSFFKTLNDDSVVEIAKVAISGGFQKLENLKLSINHKITEEGYRNFFQALDNMPNLQELDISRHFTECIKAQATTVKSLSQCVLRLPRLIRLNMLSWLLDADDIALLNVMKERHPQSKYLTILQKWILPFSPIIQK

SEQ ID NO: 65 - protein, 266 aa, Homo sapiens
MVCLKLPGGSSLAALTVTLMVLSSRLAFAGDTRPRFLELRKSECHFFNGTERVRYLDRYFHNQEEFLRFDSDVGEYRAVTELGRPVAESWNSQKDLLEQKRGRVDNYCRHNYGVGESFTVQRRVHPQVTVYPAKTQPLQHHNLLVCSVSGFYPGSIEVRWFRNGQEEKAGVVSTGLIQNGDWTFQTLVMLETVPRSGEVYTCQVEHPSVTSALTVEWRARSESAQSKMLSGVGGFVLGLLFLGAGLFIYFRNQKGHSGLQPTGFLS

SEQ ID NO: 66 - protein, 397 aa, Homo sapiens
MRSPSAAWLLGAAILLAASLSCSGTIQGTNRSSKGRSLIGKVDGTSHVTGKGVTVETVFSVDEFSASVLTGKLTTVFLPIVYTIVFVVGLPSNGMALWVFLFRTKKKHPAVIYMANLALADLLSVIWFPLKIAYHIHGNNWIYGEALCNVLIGFFYGNMYCSILFMTCLSVQRYWVIVNPMGHSRKKANIAIGISLAIWLLILLVTIPLYVVKQTIFIPALNITTCHDVLPEQLLVGDMFNYFLSLAIGVFLFPAFLTASAYVLMIRMLRSSAMDENSEKKRKRAIKLIVTVLAMYLICFTPSNLLLVVHYFLIKSQGQSHVYALYIVALCLSTLNSCIDPFVYYFVSHDFRDHAKNALLCRSVRTVKQMQVSLTSKKHSRKSSSYSSSSTTVKTSY

SEQ ID NO: 67 - protein, 374 aa, Homo sapiens
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIRKELKRKKKWDLEISLDSGHEKKVISSLQEDRHLEEELKCQEQKEEQLQEGVHRKEPQGAT

SEQ ID NO: 68 - protein, 482 aa, Homo sapiens
MASFSAETNSTDLLSQPWNEPPVILSMVILSLTFLLGLPGNGLVLWVAGLKMQRTVNTIWFLHLTLADLLCCLSLPFSLAHLALQGQWPYGRFLCKLIPSIIVLNMFASVFLLTAISLDRCLVVFKPIWCQNHRNVGMACSICGCIWVVAFVMCIPVFVYREIFTTDNHNRCGYKFGLSSSLDYPDFYGDPLENRSLENIVQPPGEMNDRLDPSSFQTNDHPWTVPTVFQPQTFQRPSADSLPRGSARLTSQNLYSNVFKPADVVSPKIPSGFPIEDHETSPLDNSDAFLSTHLKLFPSASSNSFYESELPQGFQDYYNLGQFTDDDQVPTPLVAITITRLVVGFLLPSVIMIACYSFIVFRMQRGRFAKSQSKTFRVAVVVVAVFLVCWTPYHIFGVLSLLTDPETPLGKTLMSWDHVCIALASANSCFNPFLYALLGKDFRKKARQSIQGILEAAFSEELTRSTHCPSNNVISERNSTTV

SEQ ID NO: 69 - protein, 160 aa, Homo sapiens
MLPFLFFSTLFSSIFTEAQKQYWVCNSSDASISYTYCDKMQYPISINVNPCIELKGSKGLLHIFYIPRRDLKQLYFNLYITVNTMNLPKRKEVICRGSDDDYSFCRALKGETVNTTISFSFKGIKFSKGKYKCVVEAISGSPEEMLFCLEFVILHQPNSN

SEQ ID NO: 70 - protein, 470 aa, Homo sapiens
MLRGISQLPAVATMSWVLLPVLWLIVQTQAIAIKQTPELTLHEIVCPKKLHILHKREIKNNQTEKHGKEERYEPEVQYQMILNGEEIILSLQKTKHLLGPDYTETLYSPRGEEITTKPENMEHCYYKGNILNEKNSVASISTCDGLRGYFTHHHQRYQIKPLKSTDEKEHAVFTSNQEEQDPANHTCGVKSTDGKQGPIRISRSLKSPEKEDFLRAQKYIDLYLVLDNAFYKNYNENLTLIRSFVFDVMNLLNVIYNTIDVQVALVGMEIWSDGDKIKVVPSASTTFDNFLRWHSSNLGKKIHDHAQLLSGISFNNRRVGLAASNSLCSPSSVAVIEAKKKNNVALVGVMSHELGHVLGMPDVPFNTKCPSGSCVMNQYLSSKFPKDFSTSCRAHFERYLLSQKPKCLLQAPIPTNIMTTPVCGNHLLEVGEDCDCGSPKECTNLCCEALTCKLKPGTDCGGDAPNHTTE

SEQ ID NO: 71 - protein, 373 aa, Homo sapiens
MLKRKPSNVSEKEKHQKPKRSSSFGNFDRFRNNSLSKPDDSTEAHEGDPTNGSGEQSKTSNNGGGLGKKMRAISWTMKKKVGKKYIKALSEEKDEEDGENAHPYRNSDPVIGTHTEKVSLKASDSMDSLYSGQSSSSGITSCSDGTSNRDSFRLDDDGPYSGPFCGRARVHTDFTPSPYDTDSLKIKKGDIIDIICKTPMGMWTGMLNNKVGNFKFIYVDVISEEEAAPKKIKANRRSNSKKSKTLQEFLERIHLQEYTSTLLLNGYETLEDLKDIKESHLIELNIENPDDRRRLLSAAENFLEEEIIQEQENEPEPLSLSSDISLNKSQLDDCPRDSGCYISSGNSDNGKEDLESENLSDMVHKIIITEPSD

SEQ ID NO: 72 - protein, 241 aa, Homo sapiens
MGTASRSNIARHLQTNLILFCVGAVGACTLSVTQPWYLEVDYTHEAVTIKCTFSATGCPSEQPTCLWFRYGAHQPENLCLDGCKSEADKFTVREALKENQVSLTVNRVTSNDSAIYICGIAFPSVPEARAKQTGGGTTLVVREIKLLSKELRSFLTALVSLLSVYVTGVCVAFILLSKSKSNPLRKKEIKEDSQKKKSARRIFQEIAQELYHKRHVETNQQSEKDNNTYENRRVLSNYERP

SEQ ID NO: 73 - protein, 199 aa, Homo sapiens
MSSENCFVAENSSLHPESGQENDATSPHFSTRHEGSFQVPVLCAVMNVVFITILIIALIALSVGQYNCPGQYTFSMPSDSHVSSCSEDWVGYQRKCYFISTVKRSWTSAQNACSEHGATLAVIDSEKDMNFLKRYAGREEHWVGLKKEPGHPWKWSNGKEFNNWFNVTGSDKCVFLKNTEVSSMECEKNLYWICNKPYK

SEQ ID NO: 74 - protein, 1156 aa, Homo sapiens
MSKLRMVLLEDSGSADFRRHFVNLSPFTITVVLLLSACFVTSSLGGTDKELRLVDGENKCSGRVEVKVQEEWGTVCNNGWSMEAVSVICNQLGCPTAIKAPGWANSSAGSGRIWMDHVSCRGNESALWDCKHDGWGKHSNCTHQQDAGVTCSDGSNLEMRLTRGGNMCSGRIEIKFQGRWGTVCDDNFNIDHASVICRQLECGSAVSFSGSSNFGEGSGPIWFDDLICNGNESALWNCKHQGWGKHNCDHAEDAGVICSKGADLSLRLVDGVTECSGRLEVRFQGEWGTICDDGWDSYDAAVACKQLGCPTAVTAIGRVNASKGFGHIWLDSVSCQGHEPAVWQCKHHEWGKHYCNHNEDAGVTCSDGSDLELRLRGGGSRCAGTVEVEIQRLLGKVCDRGWGLKEADVVCRQLGCGSALKTSYQVYSKIQATNTWLFLSSCNGNETSLWDCKNWQWGGLTCDHYEEAKITCSAHREPRLVGGDIPCSGRVEVKHGDTWGSICDSDFSLEAASVLCRELQCGTVVSILGGAHFGEGNGQIWAEEFQCEGHESHLSLCPVAPRPEGTCSHSRDVGVVCSRYTEIRLVNGKTPCEGRVELKTLGAWGSLCNSHWDIEDAHVLCQQLKCGVALSTPGGARFGKGNGQIWRHMFHCTGTEQHMGDCPVTALGASLCPSEQVASVICSGNQSQTLSSCNSSSLGPTRPTIPEESAVACIESGQLRLVNGGGRCAGRVEIYHEGSWGTICDDSWDLSDAHVVCRQLGCGEAINATGSAHFGEGTGPIWLDEMKCNGKESRIWQCHSHGWGQQNCRHKEDAGVICSEFMSLRLTSEASREACAGRLEVFYNGAWGTVGKSSMSETTVGVVCRQLGCADKGKINPASLDKAMSIPMWVDNVQCPKGPDTLWQCPSSPWEKRLASPSEETWITCDNKIRLQEGPTSCSGRVEIWHGGSWGTVCDDSWDLDDAQVVCQQLGCGPALKAFKEAEFGQGTGPIWLNEVKCKGNESSLWDCPARRWGHSECGHKEDAAVNCTDISVQKTPQKATTGRSSRQSSFIAVGILGVVLLAIFVALFFLTKKRRQRQRLAVSSRGENLVHQIQYREMNSCLNADDLDLMNSSENSHESADFSAAELISVSKFLPISGMEKEAILSHTEKENGNL

SEQ ID NO: 75 - protein, 307 aa, Homo sapiens
MEPSSLELPADTVQRIAAELKCHPTDERVALHLDEEDKLRHFRECFYIPKIQDLPPVDLSLVNKDENAIYFLGNSLGLQPKMVKTYLEEELDKWAKIAAYGHEVGKRPWITGDESIVGLMKDIVGANEKEIALMNALTVNLHLLMLSFFKPTPKRYKILLEAKAFPSDHYAIESQLQLHGLNIEESMRMIKPREGEETLRIEDILEVIEKEGDSIAVILFSGVHFYTGQHFNIPAITKAGQAKGCYVGFDLAHAVGNVELYLHDWGVDFACWCSYKYLNAGAGGIAGAFIHEKHAHTIKPARSEFFN

SEQ ID NO: 76 - protein, 136 aa, Homo sapiens
MRTPGPLPVLLLLLAGAPAARPTPPTCYSRMRALSQEITRDFNLLQVSEPSEPCVRYLPRLYLDIHNYCVLDKLRDFVASPPCWKVAQVDSLKDKARKLYTIMNSFCRRDLVFLLDDCNALEYPIPVTTVLPDRQR

SEQ ID NO: 77 - protein, 125 aa, Homo sapiens
MKKSGVLFLLGIILLVLIGVQGTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 78 - protein, 486 aa, Homo sapiens
MDENNGLLLLELNPPNPWDLQPRSPEELAFGEVQITYLTHACMDLKLGDKRMVFDPWLIGPAFARGWWLLHEPPSDWLERLCQADLVYISHLHSDHLSYPTLKKLAGRRPDIPIYVGNTERPVFWNLNQSGVQLTNINVVPFGIWQQVDKNLRFMILMDGVHPEMDTCIIVEYKGHKILNTVDCTRPNGGRLPMKVALMMSDFAGGASGFPMTFSGGKFTEEWKAQFIKTERKKLLNYKAWLVKNLQPRIYCPFAGYFVESHPSDKYIKETNTKNDPNELNNLIKKNSDVITWTPRPGATLDLGRMLKDPTDSKGIIEPPEGTKIYKDSWDFEPYLEILNAALGDEIFLHSSWIKEYFTWAGFKDYNLVVRMIETDEDFNPFPGGYDYLVDFLDLSFPKERPQREHPYEEIHSRVDVIRHVVKNGLLWDELYIGFQTRLQRDPDIYHHLFWNHFQIKLPLTPPNWKSFLMCCEQNGPAILQECKTT

SEQ ID NO: 79 - protein, 337 aa, Homo sapiens
MNSTCIEEQHDLDHYLFPIVYIFVIIVSIPANIGSLCVSFLQAKKESELGIYLFSLSLSDLLYALTLPLWIDYTWNKDNWTFSPALCKGSAFLMYMNFYSSTAFLTCIAVDRYLAVVYPLKFFFLRTRRFALMVSLSIWILETIFNAVMLWEDETVVEYCDAEKSNFTLCYDKYPLEKWQINLNLFRTCTGYAIPLVTILICNRKVYQAVRHNKATENKEKKRIIKLLVSITVTFVLCFTPFHVMLLIRCILEHAVNFEDHSNSGKRTYTMYRITVALTSLNCVADPILYCFVTETGRYDMWNILKFCTGRCNTSQRQRKRILSVSTKDTMELEVLE

SEQ ID NO: 80 - protein, 381 aa, Homo sapiens
MHLLAILFCALWSAVLAENSDDYDLMYVNLDNEIDNGLHPTEDPTPCDCGQEHSEWDKLFIMLENSQMRERMLLQATDDVLRGELQRLREELGRLAESLARPCAPGAPAEARLTSALDELLQATRDAGRRLARMEGAEAQRPEEAGRALAAVLEELRQTRADLHAVQGWAARSWLPAGCETAILFPMRSKKIFGSVHPVRPMRLESFSACIWVKATDVLNKTILFSYGTKRNPYEIQLYLSYQSIVFVVGGEENKLVAEAMVSLGRWTHLCGTWNSEEGLTSLWVNGELAATTVEMATGHIVPEGGILQIGQEKNGCCVGGGFDETLAFSGRLTGFNIWDSVLSNEEIRETGGAESCHIRGNIVGWGVTEIQPHGGAQYVS

SEQ ID NO: 81 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 82 - protein, 285 aa, Homo sapiens
MVMRPLWSLLLWEALLPITVTGAQVLSKVGGSVLLVAARPPGFQVREAIWRSLWPSEELLATFFRGSLETLYHSRFLGRAQLHSNLSLELGPLESGDSGNFSVLMVDTRGQPWTQTLQLKVYDAVPRPVVQVFIAVERDAQPSKTCQVFLSCWAPNISEITYSWRRETTMDFGMEPHSLFTDGQVLSISLGPGDRDVAYSCIVSNPVSWDLATVTPWDSCHHEAAPGKASYKDVLLVVVPVSLLLMLVTLFSAWHWCPCSGKKKKDVHADRVGPETENPLVQDLP

SEQ ID NO: 83 - protein, 510 aa, Homo sapiens
MEDTKESNVKTFCSKNILAILGFSSIIAVIALLAVGLTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHSTYVFLMVLFSLVLFTVAIIGLLIFHKPSYFWKDMV

SEQ ID NO: 84 - protein, 355 aa, Homo sapiens
METPNTTEDYDTTTEFDYGDATPCQKVNERAFGAQLLPPLYSLVFVIGLVGNILVVLVLVQYKRLKNMTSIYLLNLAISDLLFLFTLPFWIDYKLKDDWVFGDAMCKILSGFYYTGLYSEIFFIILLTIDRYLAIVHAVFALRARTVTFGVITSIIIWALAILASMPGLYFSKTQWEFTHHTCSLHFPHESLREWKLFQALKLNLFGLVLPLLVMIICYTGIIKILLRRPNEKKSKAVRLIFVIMIIFFLFWTPYNLTILISVFQDFLFTHECEQSRHLDLAVQVTEVIAYTHCCVNPVIYAFVGERFRKYLRQLFHRRVAVHLVKWLPFLSVDRLERVSSTSPSTGEHELSAGF

SEQ ID NO: 85 - DNA, 63 bp, Artificial Sequence
  Description of Artificial Sequence Synthetic primer
ggccagtgaattgtaatacgactcactatagggaggcggtttttttttttttttttttttttt

(19) United States
(12) Patent Application Publication
(10) Pub. No.: US 2006/0194238 A1
(43) Pub. Date: Aug. 31, 2006

(54) Peripheral neuropathy diagnosis

(76) Inventors: Norman Latov, Irvington, NY (US); Susanne Renaud, Basel (DE)

Correspondence Address: VENABLE LLP, P.O. BOX 34385, WASHINGTON, DC, 20045-9998, US

(21) Appl. No.: 11/363,151

(22) Filed: Feb. 28, 2006

Related U.S. Application Data
(60) Provisional application No. 60/657,122, filed on Feb. 28, 2005.

(51) Int. Cl. C12Q 1/68; G01N 33/53

(52) U.S. Cl. 435/6; 435/7.1

(57) ABSTRACT

Genes whose expression is correlated with the presence of CIDP or vasculitic neuropathy are disclosed. Probes and sets of nucleic acids and proteins specific for these genes are described, as are molecular and immunological methods for aiding in the diagnosis of these disease conditions in a subject.

This application claims the benefit of the filing date of U.S. Provisional Application Ser. No. 60/657,122, filed Feb. 28, 2005, whose disclosure is entirely incorporated by reference herein. This application is related to co-pending U.S. application, attorney docket number 67366-228224, filed herewith.

### FIELD OF THE INVENTION

The present invention relates, e.g., to a composition comprising a plurality of nucleic acid probes for use in research and diagnostic applications.

### BACKGROUND INFORMATION

Chronic inflammatory demyelinating polyradiculoneuropathy (CIDP) is an autoimmune disease that targets myelin sheaths, specifically in the peripheral nerves, and causes progressive weakness and sensory loss. Vasculitis is caused by inflammation of the blood vessel walls. When the blood vessels in the nerves are affected, it is referred to as vasculitic neuropathy.

Both CIDP and vasculitic neuropathy cause peripheral neuropathy which is manifest by sensory loss, weakness, or pain, alone or in combination, in the arms, legs, or other parts of the body. Both can cause a symmetric or multifocal neuropathy and affect the proximal or distal muscles. There are many other causes of neuropathy besides CIDP and vasculitis, but in one quarter to one third of neuropathies, no cause can be found, and the neuropathy is called idiopathic. This is due, in part, to the lack of reliable tests for many causes of neuropathy.

CIDP is currently diagnosed based on the clinical presentation, evidence for demyelination on electrodiagnostic studies or pathological studies of biopsied nerves, and elimination of other known causes of neuropathy such as genetic defects, osteosclerotic myeloma, or IgM monoclonal gammopathy. There is currently no definitive test, and the diagnosis can be missed, especially in atypical cases or in sensory CIDP where the electrodiagnostic tests are less reliable. Such cases may be difficult to distinguish from vasculitic neuropathy. Nerve biopsy is done in cases where the diagnosis is uncertain, but its usefulness is limited by its relative insensitivity and the need for quantitative morphological analysis which is only available in a small number of academic centers. For further discussions about properties of, or current diagnostic methods for, CIDP, see, e.g., Dyck et al. (1975) Mayo Clin. Proc. 50, 621-637; Latov (2002) Neurology 59, S2-S6; Berger et al. (2003) J. Peripher. Nerv. Sys. 8, 282-284; Ad Hoc Subcommittee of the AAN (1991); Barohn et al. (1989) Arch. Neurol. 46, 878-884; Bouchard et al. (1999) Neurology 52, 498-503).

In vasculitic neuropathy, the diagnosis can be easily missed if the vasculitis selectively affects the peripheral nerves, and there is no involvement of other organs. In such cases, the diagnosis can currently only be made by nerve or nerve and muscle biopsy. For a further discussion of classification and treatment of vasculitic neuropathy, see Schaublin et al. (2005) Neurology 4, 853-65.

Both CIDP and vasculitic neuropathy are treatable conditions, and early intervention can prevent permanent damage and disability. Therefore, it would be desirable to develop improved methods for accurately diagnosing these conditions., e.g. in subjects with neuropathy of otherwise unknown etiology who are suspected of having CIDP or vasculitic neuropathy.

Parallel profiling of global gene expression levels based on microarray technologies has emerged as a powerful tool to identify markers associated with particular disease conditions. See, e.g., Duggin et al. (1999) Nat. Genet. 21 (1 Suppl;), 10-14 or Lockhart et al. (1996) Nat. Biotech. 14, 1675-1680. The present inventors have analyzed gene expression profiles of patients diagnosed with CIDP or vasculitic neuropathy, and have identified genes whose over-expression or under-expression is correlated with these disease conditions. Combinations comprising probes specific for these genes or their gene products can be used in, e.g., diagnostic and experimental methods.

### BRIEF DESCRIPTION OF THE DRAWINGS

FIG. 1 shows RT real-time PCR in the analysis of expression in nerves of CIDP patients. The up-regulation of IL7, TAC, SCD, CD69 and down regulation of DCXR gene expression genes in CIDP versus normal nerve biopsy samples (NN), which had been observed in studies with gene arrays, was confirmed here by RT real-time PCR. A good correlation between fold changes and relative quantities was observed for all genes analyzed.

FIG. 2 shows RT real-time PCR in the analysis of expression in nerves of patients suffering from vasculitic neuropathy. The up regulation of IL7, PTX3, CD69, HAMP and down regulation of CRYAB in vasculitic nerve (VAS) compared to NN, which had been observed in studies with gene arrays, was confirmed here by RT real-time PCR.

### DESCRIPTION OF THE INVENTION

The present invention relates, e.g., to the identification of genes and gene products (molecular markers, disease markers) whose expression (up-regulation or down-regulation), compared to a baseline value, is correlated with the presence of CIDP or vasculitic neuropathy. “Up-regulation” or “over-expression” of a gene, as used herein, can refer either to an increased expression of a gene (to generate an mRNA or protein gene product), e.g., in nerve tissue, or to an increased amount of expression brought about by the migration of inflammatory cells into the affected area.

As used herein, a “baseline value” includes, e.g., the expression in normal tissue (e.g. the same type of tissue as the tested tissue, such as normal nerve, or skin) from normal subjects. If desired, a pool of the same tissues from normal subjects may be used. The pooled values may be either commercially available or otherwise derived. Alternatively, the baseline value may be the expression in comparable tissues from patients exhibiting other disease conditions that do not affect the same tissue; in the Examples herein, the comparison is done to nerves from control patients with intact nerve suffering from myopathy, muscular dystrophy or dermatomyositis. Alternatively, the baseline may be the expression of one or more housekeeping genes (e.g., constitutively expressed genes) from the patient being studied, as internal controls. Suitable genes which can be used as such internal (endogenous) controls will be evident to a skilled worker; among the genes which can be used are: GAPDH (glyceraldehydes-3-phosphate dehydrogenase) and beta-actin. If desired, housekeeping genes from nerves may be used, e.g. S100 protein, which is specific for Schwann cells, or GFAP (glial fibriallary acidic protein). Any of these types of baseline values may be available in a database compiled from the values.

For CIDP, about 123 molecular markers are identified herein that are expressed in a significantly altered amount compared to a baseline value. About 101 genes are up-regulated, and about 22 are down-regulated (greater than twofold change and p<0.05). See, e.g., Table 3 (up-regulated) and Table 4 (down-regulated). Of course, other genes, as well, may be differentially expressed in the disease. The 15 most highly over-expressed genes are summarized in Table 5. Polynucleotides corresponding to these 15 genes are represented by SEQ ID NOs: 1-16; and the corresponding polypeptides are represented by SEQ ID NOs 17-32. The terms “polynucleotide” and “oligonucleotide” are used interchangeably herein, as are the terms “polypeptide” and “peptide.”

For vasculitic neuropathy, at least 244 genes are identified herein that are expressed in a significantly altered amount compared to a baseline value. About 163 genes are up-regulated and about 81 are down-regulated (greater than twofold change and p<0.05). Table 6 shows marker genes with putative functions in immunity; all except the last two genes in the Table (CXCR2 etc. and CD24A) are up-regulated. In general, the discussion herein with regard to Table 6 concerns the up-regulated genes. Of course, other genes, as well, may be differentially regulated in the disease. The 30 most highly over-expressed genes (with about a 5-fold or greater increase) are summarized in Table 7. Many of the genes in this Table are not involved in immune functions, and thus are not shown in Table 6. Although not listed in Table 7, TAC1 is also over-expressed, by about 5-fold. Polynucleotides corresponding to these 30 genes are represented by SEQ ID NOs: 4, 6, 7, 13, 14, or 33-58; and the corresponding polypeptides are represented by SEQ ID NOs 20, 22, 23, 29, 30, or 59-84.

Twenty four of the markers shown as being aberrantly expressed in CIDP (Tables 3 and 4) are also shown to be aberrantly expressed in vasculitic neuropathy (Table 6). Four of the markers indicated in Table 5 as being highly up-regulated in CIDP are also indicated in Table 7 as being highly up-regulated in vasculitic neuropathy (AIF1, MSR1, CLCA2 and PCSK1). Some of the markers indicated in Table 7 as being particularly highly expressed in vasculitic neuropathy are not shown in Table 6, as Table 6 only includes genes with putative functions in immunity, whereas Table 7 also contains up-regulated genes that have no known immune functions. Many of the up-regulated genes in Tables 6 and 7 reflect the presence of inflammatory cells which have invaded the affected area.

It is notable that three of the genes which are highly over-expressed in CIDP (SCD, NQ01 and NR1D1 ) are not over-expressed in vasculitic neuropathy. Therefore, expression of one or more of these three genes can be useful for distinguishing between the conditions. For example, a finding that one or more (e.g. two or more, or all three) of these genes is over-expressed in a sample from a patient (in addition to the over-expression of one or more additional genes, such as TAC1 or A1F1) indicates that the patient is likely to be suffering from (has an increased likelihood of suffering from) CIDP rather than from vasculitic neuropathy; and, conversely, the absence of over-expression of one or more of these three genes indicates that the subject likely does not suffer from CIDP. By using a suitable combination of genes that are over-expressed and/or under-expressed in CIDP and/or vasculitic neuropathy, one can determine if a subject is likely to be suffering from CIPD or vasculitic neuritis.

Some of the above-mentioned markers are identified in Renaud et al. (2005) Journal of Neuroimmunology 159, 203-214, which is incorporated by reference herein in its entirety.

The molecular markers identified herein can serve as the basis for a variety of assays to distinguish among the various types of peripheral neuropathy. For example, suitable combinations of nucleic acid probes corresponding to one or more of the genes, and/or antibodies specific for proteins encoded by the genes, can be used to analyze a sample from a subject suspected of having CIDP or vasculitic neuropathy, in order aid in the diagnosis of the disease condition; to follow the course of the disease; to evaluate the response to therapeutic agents; etc. Any suitable number of molecules (e.g. nucleic acid probes, antibodies, etc) corresponding to the identified genes, in any combination, can be used in compositions and methods of the invention. Generally, an analysis of the expression of a large number of genes provides a more accurate identification of a disease condition than does the expression of a subset of those genes. That is, as increasing numbers of markers for a given disease condition are shown to be over-expressed in a subject, the likelihood that the subject suffers from that disease increases; and the identification (diagnosis) of the disease condition becomes more certain. Although the term “diagnosis” is sometimes used herein, it is to be understood that an assay for expressed gene markers cannot, in itself, provide a definitive diagnosis, absent the consideration of other factors. The identification of markers for CIDP and vasculitic neuropathy can also aid in the identification of targets for therapeutic intervention, or of therapeutic agents for treating the disease conditions. Furthermore, the identification of genes whose expression is correlated with these conditions can also provide a basis for explaining the molecular or metabolic processes involved in pathogenesis, and thus can be used as research tools.

Advantages of assaying for specific markers in addition to, or instead of, conventional diagnostic methods include: (1) In cases where a nerve biopsy is obtained for making a diagnosis, current methods are based on morphological examination, which is relatively insensitive. Being able to measure molecular markers that are indicative of the disease allows for a more quantitative and sensitive test. (2) Having the ability to use sensitive molecular markers rather than morphological examination makes it possible to make a diagnosis more reliably and using a smaller amount of tissue. Currently, most biopsies use the sural nerve as it is sufficiently large for pathological studies, is purely sensory, and enervates only the lateral part of the foot, so that the functional loss is limited. Having the ability to use a smaller amount of tissue makes it possible to use a small piece of any nerve that is accessible, including skin which is known to contain myelinated nerve fibers. Methods of the invention are less cumbersome, time-consuming and expensive than are currently employed methods.

One aspect of the invention is a composition (combination) comprising one or a plurality of (e.g. at least about 5, 10, 15, 25, 50, 75, 100, 200, 300, 400 or more) isolated nucleic acids of at least about 8 contiguous nucleotides (e.g., at least about 12, 15, 25, 35, 50 or 75 contiguous nucleotides), selected from nucleic acids that correspond to different genes listed in Tables 3, 4, 5, 6 and/or 7. Any combination of those nucleic acids may be present in a composition of the invention. A composition of the invention preferably comprises no more than about 1×10⁶ (e.g., no more than about 500,000; 200,000; 100,000; 50,000; 25,000; 14,000; 13000; 12,000; 11,000; 10,000; 9,000; 8,000; 7,000; 6,000; 5,000, 4,000; 3,000; 2,000; 1,000; 500; 250; 150; 75 or 50) total isolated nucleic acids.

In embodiments of the invention, compositions can comprise nucleic acids that consist essentially of about 15-50 nucleotides (nt); comprise at least about 15 nt; comprise at least about 50 nt; and/or are cDNAs.

The composition may be used, e.g., to detect the expression of genes associated with CIDP or with vasculitis (e.g. vasculitic neuropathy).

As used herein, the term “isolated” nucleic acid (or polypeptide, or antibody) refers to a nucleic acid (or polypeptide, or antibody) that is in a form other than it occurs in nature, for example in a buffer, in a dry form-awaiting reconstitution, as part of an array, a kit or a pharmaceutical composition, etc. The term an “isolated” nucleic acid or protein does not include a cell extract (e.g. a crude or semi-purified cell extract).

As used herein, the term “about,” when referring to the size of a biological molecule, includes a size that is up to 20% larger or smaller than the size of the molecule. For example, a nucleic acid that is about 50 nt can range from 40 to 60 nts.

Nucleic acids or proteins that “correspond to” a gene include nucleic acids or proteins that are expressed by the gene, or active fragments or variants of the expressed nucleic acids or proteins, or complements of the nucleic acids or fragments, etc. Untranslated sequences of the genes are included. Only one strand of each nucleic acid or polynucletide is shown, but the complementary strand is understood to be included by any reference to the displayed strand. A “complement,” as used herein, is a complete (full-length) complementary strand (with no mismatches) of a single strand nucleic acid. More than one nucleic acid corresponding to a given gene can be present in a composition of the invention. For example, active fragments from two or more regions of a nucleic acid, all of which correspond to the gene, can be present.

The individual sequences of nucleic acids and proteins in the compositions and methods of the invention were publicly available at the time the invention was made. However, the relationship between the expression of these molecules and CIDP or vasculitic neuropathy had not previously been observed; and the particular combinations of molecules in the compositions of the invention had not been disclosed or suggested.

The GenBank accession numbers of the nucleic acids sequences (and proteins translated from them) which are identified herein as being markers for CIDP or vasculitic neuropathy are provided in Tables 3-7. Sequences corresponding to the most highly up-regulated genes, as presented in Tables 5 and 7, are provided in the Sequence Listing attached hereto. Sequences which are not provided in the Sequence Listing can be readily obtained by referring to the GenBank Accession Numbers.

Probes obtained from Affymetrix were used in the experiments described herein to identify the molecular markers of the invention. Some of those probes may represent full-length coding sequences, and others may be less than full-length. Full-length nucleic acid sequences (e.g., full-length coding sequences or genomic sequences) that correspond to the less than full-length probes can be readily obtained, using conventional methods to mine Genbank sequences.

One aspect of the invention is a composition comprising at least two isolated nucleic acids of at least about 15 contiguous nucleotides selected from nucleic acids that correspond to genes #1-15 from Table 5. The composition may contain nucleic acids corresponding to any combination of two or more of the genes in the Table.

In one embodiment, the nucleic acids correspond to (a) one or more (e.g., two or more, or all three) of the genes which are shown herein to be expressed highly in CIDP but not in vascular neuropathy—genes #2 (NR1D1), #3 (SCD), and #9 (NQ01)—and (b) one or more of the remaining genes listed in Table 5 (the “remaining” genes in this composition do not include the genes in (a)) and/or the remaining CIDP-specific genes listed in Tables 3 and/or 4. The number of remaining genes in Table 5 can be, e.g., five or ten. In one embodiment of the invention, the genes from set (b) are selected from gene #1 (TAC1), gene #4 (AIF1) and gene #12 (CLCA2), preferably from TAC2 and AIF1. In another embodiment, the genes in (b) are selected from gene #6 (MSR1) and gene #13 (PCKS1), or are selected from TAC1, AIF1, CLCA2, MSR1 and PCKS1. One embodiment of the invention is a composition that comprises nucleic acids which correspond to SCD, NQO1, NR1D1, TAC1, AIF1, MSR1, PCKS1 and CLCA2.

Another embodiment is a composition which comprises any combination of nucleic acids corresponding to genes listed in Table 5, as described above, which further comprises one or more nucleic acids corresponding to the remaining genes in Tables 6 and/or 7. The number of different genes in Table 7 can be, e.g., about 10, 20 or up to all of the remaining genes.

In cases in which a subject is suspected of having CIDP, and not vasculitic or any other type of neuropathy, a composition comprising nucleic acids corresponding to NQO1 and/or NRD1 and, optionally, SCD can be used to help confirm, or increase the likelihood, that the subject has CIDP.

Any composition of the invention may also contain one or more internal control nucleic acids, such as nucleic acids corresponding to constitutively expressed genes. Suitable controls will be evident to the skilled worker. They include, e.g., actin (e.g. beta-actin), GAPDH, S100 protein, GFAP, or the like.

Another aspect of the invention is a composition comprising two or more isolated nucleic acids of at least about 15 contiguous nucleotides selected from nucleic acids that correspond to genes #1-31 from Table 7. The combination may contain nucleic acids corresponding to any combination of two or more genes in the table.

One embodiment of the invention is such a composition, wherein the nucleic acids correspond to

(a) one, two, three, four or five of genes #1-5 in Table 7; and/or

(b) one, two, three, four or five of genes #6-10 in Table 7; and/or

(c) one, two, three, four or five of genes #11-15 in Table 7; and/or

(d) one, two, three, four or five of genes #16-20 in Table 7; and/or

(e) one, two, three, four or five of genes #21-25 in Table 7; and/or

(f) one, two, three, four or five of genes #25-30 in Table 7,

wherein if a nucleic acid that corresponds SCD is present, a nucleic acid corresponding to at least one other gene must also be present. (In compositions of the invention, if a nucleic acid that corresponds to CD86 is present, a nucleic acid corresponding to at least one other gene must also be present.) Preferably, the composition comprises nucleic acids corresponding to at least two (e.g., at least about 3, 5, 10, or up to all ) different genes.

Nucleic acids which correspond to the genes in Table 5 include:

(a) nucleic acids that comprise the sequences of SEQ ID NOs 1-16;

(b) nucleic acids that comprise sequences which are at least about 85% (e.g 90%, 95%, 98%) identical to the contiguous sequences in (a);

(c) nucleic acids that comprise sequences encoding polypeptides represented by SEQ ID NOs: 17-32;

(d) nucleic acids that comprise sequences of active fragments of the nucleic acids of (a), (b), and/or (c);

(e) nucleic acids that comprise complete complements of the sequences of any of (a), (b), (c), and/or (d); and/or

(f) nucleic acids that comprise sequences of active variants of the nucleic acids of (a), (b), (c), (d), and/or (e).

Each of the nucleic acids noted above (e.g. having the mentioned percent identity, fragments of the longer molecules, etc.) can hybridize under conditions of high stringency to nucleic acids represented by SEQ ID NO's 1-16, or to complete complements thereof.

Nucleic acids which correspond to the genes in Table 7 include

(a) nucleic acids that comprise the sequences of SEQ ID NOs: 4, 6, 7, 13, 14, or 33-58;

(b) nucleic acids that comprise sequences which are at least about 85% (e.g. 90%, 95%, 98%) identical to the contiguous sequences in (a);

(c) nucleic acids that comprise sequences encoding polypeptides represented by SEQ ID NOs: 20, 22, 23, 29, 30, or 59-84;

(d) nucleic acids that comprise sequences of active fragments of the nucleic acids of (a), (b), and/or (c);

(e) nucleic acids that comprise complete complements of the sequences of any of (a), (b), (c), and/or (d); and/or

(f) nucleic acids that comprise sequences of active variants of the nucleic acids of (a), (b), (c), (d), and/or (e).

Each of the nucleic acids noted above (e.g. having the mentioned percent identity, fragments of the longer molecules, etc.) can hybridize under conditions of high stringency to nucleic acids represented SEQ ID NO's SEQ ID NOs: 4, 6, 7, 13, 14, or 33-58, or to complete complements thereof.

In embodiments of the invention, the composition comprises nucleic acids which correspond to genes from Table 5 and/or from Table 7, wherein the nucleic acids are active fragments of about 15 to about 50 contiguous nucleotides from SEQ ID NOs: 1-16, or SEQ ID NOs: 4, 6, 7, 13, 14 or 33-58, respectively.

The nucleic acids discussed above, and derivatives thereof, can be used as probes to identify (e.g., by hybridization assays) polynucleotides whose expression is altered, compared to a baseline value, in CIDP or vasculitic neuropathy.

Compositions of the invention may comprise any combination of, e.g., at least about 1, 2, 5, 10, 15, 20, 25, 50, 75 or 100 or more of the mentioned nucleic acids and/or fragments. A nucleic acid composition of the invention may comprise, consist essentially of, or consist of, a total of, e.g., about 1, 2, 5, 10, 15, 20, 25, 50, 60, 70, 100, 150, 250, 500, 750, 1,000, 2,000, 3,000, 5,000, 7,000; 8,000; 9,000; 10,000, 11,000; 12,000; 13,000; 14,000; 15,000; 25,000, 50,000, 100,000, 200,000, 500,000, 1×10⁶, or more isolated nucleic acids. The term “consisting essentially of,” in this context, refers to a value intermediate between the specific number of the mentioned elements (here, nucleic acids) encompassed by the term “consisting of” and the large number encompassed by the term “comprising.” A nucleic acid composition of the invention preferably comprises no more than a total of, e.g., about 1×10⁶ (e.g., no more than about 500,000; 200,000; 100,000; 50,000; 25,000; 14,000; 13,000; 12,000; 11,000; 10,000; 9,000; 8,000; 7,000; 6,000; 5,000, 4,000; 3,000; 2,000; 1,000; 750; 500; 300; 200; 150; 100; 70; 60; 50; 25; 20; 15; 10; 5; 2; or 1) isolated nucleic acids.

The nucleic acid compositions of the invention may be in the form of an aqueous solution, or the nucleic acids in the composition may be immobilized on a substrate. In some compositions of the invention, the isolated nucleic acids are in an array, such as a microarray, e.g., they are hybridizable elements on an array, such as a microarray. A nucleic acid array may further comprise, bound (e.g., bound specifically) to one or more nucleic acids of the array, polynucleotides from a sample representing expressed genes. In general, as used herein, the term “nucleic acid” refers to a probe, whereas the term “polynucleotide” refers to an expression product of a gene, or a derivative of such an expression product (e.g. an amplified product). In one embodiment, the nucleic acids in an array and the polynucleotides from a sample representing expressed genes have been subjected to nucleic acid hybridization under high stringency conditions (such that nucleic acids of the array that are specific for particular polynucleotides from the sample are specifically hybridized to those polynucleotides). Another embodiment is a composition comprising one or a plurality of isolated nucleic acids, each of which hybridizes specifically under high stringency conditions to part or all of a coding sequence whose expression reflects (is indicative of, is correlated with) the presence or absence of CIDP or vasculitic neuropathy.

Sequences “corresponding to” a gene, or “specific for” a gene include sequences that are substantially similar to (e.g., hybridize under conditions of high stringency to) one of the strands of the double stranded form of that gene. By hybridizing “specifically” is meant herein that two components (e.g. an expressed gene or polynucleotide and a nucleic acid probe) bind selectively to each other and not generally to other components unintended for binding to the subject components. The parameters required to achieve specific interactions can be determined routinely, using conventional methods in the art.

In the present application, the term “nucleic acid” (e.g., with reference to probe molecules) refers both to DNA (including cDNA) and RNA, as well as DNA-like or RNA-like materials, such as branched DNAs, peptide nucleic acids (PNA) or locked nucleic acids (LNA). Nucleic acid probes for gene expression analysis include those comprising ribonucleotides, deoxyribonucleotides, both, and/or their analogues. Nucleic acids of the invention include double stranded and partially or completely single stranded molecules. In a preferred embodiment, probes for gene expression comprise single stranded nucleic acid molecules that are complementary to an mRNA target expressed by a gene of interest, or that are complementary to the opposite strand (e.g., complementary to a first strand cDNA generated from the mRNA).

Some of the polynucleotide sequences referred to herein may be partial cDNAs, gene fragments, or ESTs. For purposes of the analysis, it is not necessary that the full length sequence be known, as those of skill in the art will know how to obtain the full length sequence using the sequence of a given fragment or EST and known data mining, bioinformatic, and DNA sequencing methodologies without undue experimentation. If desired, the skilled artisan can subsequently select as a probe a nucleic acid that is longer than the initial gene fragment or EST, or a suitable fragment selected from that extended sequence. Since some of the probe sequences are identified solely based on expression levels, it is not essential to know a priori the function of a particular gene.

The present invention includes a variety of active variants of nucleic acids. For example, nucleic acid probes can be sequence variants of the sequences described herein (e.g., they can include nucleotide substitutions, small insertions or deletions, nucleotide analogues, etc.); or they can be chemical variants (e.g., they can contain chemical derivatives); or they can be length variants. An “active variant,” as used herein, is a variant that retains a measurable amount of an activity of the starting material. For example, an active variant of a nucleic acid probe retains an adequate ability to hybridize specifically to a complementary DNA strand (or mRNA) in a test sample, under suitable hybridization conditions. Preferably, an active variant of a nucleic acid probe also exhibits adequate resistance to nucleases and stability in the hybridization protocols employed. DNA or RNA may be made more resistant to nuclease degradation, e.g., by incorporating modified nucleosides (e.g., 2′-0-methylribose or 1′-α-anomers), or by modifying internucleoside linkages (e.g., methylphosphonates or phosphorothioates), as described below.

With regard to sequence variants, the invention includes nucleic acid probes which exhibit variations in sequence compared to the wild type sequence, provided the probe retains the ability to hybridize specifically to the polynucleotide to which it corresponds (e.g., to the nucleic acid from which it is derived, or a complement thereof). For example, small deletions, insertions, substitutions, rearrangements etc. are tolerated. The sequence changes may be introduced artificially, or they may be naturally occurring, e.g., changes reflecting degeneracy of the genetic code, allelic variants, species homologues, etc.

Nucleotide analogues can be incorporated into the nucleic acids by methods well known in the art. The only requirement is that the incorporated nucleotide analogues must serve to base pair with target polynucleotide sequences. For example, certain guanine nucleotides can be substituted with hypoxanthine which base pairs with cytosine residues. However, these base pairs are less stable than those between guanine and cytosine. Alternatively, adenine nucleotides can be substituted with 2,6-diaminopurine which can form stronger base pairs than those between adenine and-thymidine.

The invention also relates to nucleic acid probes that are at least about 70%, 75%, 80%, 85%, 90%, 95%, 98% or 99% identical in sequence over their entire length to a polynucleotide target of interest, or to a complement thereof. Conventional algorithms can be used to determine the percent identity or complementarity, e.g., as described by Lipman and Pearson (Proc. Natl Acad Sci 80:726-730,1983) or Martinez/Needleman-Wunsch (Nucl Acid Research 11:4629-4634, 1983).

The invention also relates to nucleic acid probes that hybridize specifically to corresponding target polynucleotides, e.g., under conditions of high stringency. Some nucleic acid probes may not hybridize effectively under hybridization conditions due to secondary structure. To optimize probe hybridization, the probe sequences may be examined using a computer algorithm to identify portions of genes without potential secondary structure. Such computer algorithms are well known in the art, such as OLIGO 4.06 Primer Analysis Software (National Biosciences, Plymouth, Minn.) or LASERGENE software (DNASTAR, Madison, Wis.); MACDASLS software (Hitachi Software Engineering Co, Std. South San Francisco, Calif.) and the like. These programs can search nucleotide sequences to identify stem loop structures and tandem repeats and to analyze G+C content of the sequence (those sequences with a G+C content greater than 60% are excluded). Alternatively, the probes can be optimized by trial and error. Experiments can be performed to determine whether probes and complementary target polynucleotides hybridize optimally under experimental conditions.

With regard to chemical variants, the nucleic acids can include nucleotides that have been derivatized chemically or enzymatically. Typical chemical modifications include derivatization with acyl, alkyl, aryl or amino groups. Suitable modified base moieties include, for example, 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-ω-thiouridine, 5-carboxymethyl-aminomethyl uracil, dihydrouracil, β-D-galactosylqueosine, inosine, N6-isopentenyladenine, 1-methylguanine, 3-methyl-cytosine, 5-methylcytosine, N6-adenine, 7-methylguanine, 5-methylaminomethyluracil, 5-methoxyamino-methyl-2-thiouracil, β-D-mannosylqueosine, 5-methoxy-carboxymethyluracil, 5-methoxyuracil-2-methylthio-N6-iso-pentenyladenine, uracil-5-oxyacetic acid, butoxosine, pseudouracil, queuosine, 2-thio-cytosine, 5-methyl-2-thiouracil, 2-thiouracil, 4-thiouracil, 5-methyluracil, uracil-5-oxyacetic acid methylester, uracil-t-oxyacetic acid, 5-methyl-2-thiouracil, 3(3-amino-3-N-2-carboxypropyl) uracil and 2,6-diaminopurine.

The nucleic acid may comprise at least one modified sugar moiety including, but not limited, to arabinose, 2-fluoroarabinose, xylulose, and hexose.

The nucleic acid may comprise a modified phosphate backbone synthesized from one or more nucleotides having, for example, one of the following structures: a phosphorothioate, a phosphoridothioate, a phosphoramidothioate, a phosphoramidate, a phosphordiimidate, a methylphosphonate, an alkyl phosphotriester, 3′-aminopropyl and a formacetal or analog thereof.

The nucleic acid may be an α-anomeric oligonucleotide which forms specific double-stranded hybrids with complementary RNA in which, contrary to the usual β-units, the strands run parallel to each other (Gautier et al. (1987), Nucl. Acids Res. 15:6625-6641).

The nucleic acid may be conjugated to another molecule, e.g., a peptide, a hybridization-triggered cross-linking agent, a hybridization-triggered cleavage agent, etc., all of which are well-known in the art.

With regard to length variants (active fragments), those skilled in the art will appreciate that a probe of choice for a particular gene can be the full length coding sequence or any fragment thereof having generally at least about 8 or at least about 15 nucleotides. When the full length sequence is known, the practitioner can select any appropriate fragment of that sequence, using conventional methods. In some embodiments, multiple probes, corresponding to different portions of a given SEQ ID (molecular marker) of the invention, are used. For example, probes representing about 10 non-overlapping 20-mers can be selected from a 200-mer sequence. Thus, for example, if each of the 15 molecular markers for CIDP listed in Table 5 is represented by 10 probes, the total number of the probes corresponding to the molecular markers in the composition (e.g., in a microarray) will be 150. A skilled worker can design a suitable selection of overlapping or non-overlapping probes corresponding to each expressed polynucleotide of interest, without undue experimentation.

A nucleic acid probe of the invention can be of any suitable length. The size of the DNA sequence of interest may vary, and is preferably from about 8 to about 10,000 nucleotides, e.g. from about 50 to about 3,500 nucleotides. In some embodiments, full-length coding sequences are preferred. In others, the nucleic acids range from about 15 to about 200 nucleotides, preferably from about 50 to about 80 nucleotides. All ranges provided herein include the end point values. Any nucleic acid that can uniquely identify a polynucleotide of the invention (e.g., that can hybridize to it specifically, under high stringency conditions) is included in the invention. In general, a nucleic acid comprising at least about 8, 10, 15, 20, 25 or 50 or more contiguous nucleotides contains sufficient information to specify uniquely a gene of a mammalian (e.g., human) genome. Practically, larger oligonucleotides are often used as probes.

Nucleic acid probes (e.g., oligonucleotides) of this invention may be synthesized, in whole or in part, by standard synthetic methods known in the art. See, e.g., Caruthers et al. (1980) Nucleic. Acids Symp. Ser. (2) 215-233; Stein et al. (1998), Nucl. Acids Res. 16, 3209; and Sarin et al. (1988), Proc. Natl. Acad. Sci. U.S.A 85, 7448-7451. An automated synthesizer (such as those commercially available from Biosearch, Applied Biosystems) may be used. cDNA probes can be cloned and isolated by conventional methods; can be isolated from pre-existing clones, such as those from Incyte as described herein; or can be prepared by a combination of conventional synthetic methods.

A composition comprising nucleic acids of the invention can take any of a variety of forms. For example, the nucleic acids can be free in a solution (e.g., an aqueous solution), and can, e.g., be subjected to hybridization in solution to polynucleotides from a sample of interest, or used as primers for PCR amplification. Alternatively, the nucleic acids can be in the form of an array. The term “array” as used herein means an ordered arrangement of addressable, accessible, spatially discrete or identifiable, molecules disposed on a surface. The molecules in the array can be hybridizable elements (e.g., nucleic acids) or reactive elements (e.g., antibodies). Arrays can comprise any number of sites that comprise probes, from about 5 to, in the case of a microarray, tens to hundreds of thousands or more.

Any of a variety of suitable, compatible surfaces can be used for arrays in conjunction with this invention. The surface (usually a solid, preferably a suitable rigid or semi-rigid support) can be any of a variety of organic or inorganic materials or combinations thereof, including, merely by way of example, plastics such as polypropylene or polystyrene; ceramic; silicon; (fused) silica, quartz or glass, which can have the thickness of, for example, a glass microscope slide or a glass cover slip; paper, such as filter paper; diazotized cellulose; nitrocellulose filters; nylon membrane; or polyacrylamide gel pad. Substrates that are transparent to light are useful when the method of performing an assay involves optical detection. Suitable surfaces include membranes, filters, chips, slides, wafers, fibers, magnetic or nonmagnetic beads, gels, tubing, plates, polymers, microparticles, capillaries, or the like. The substrate can have a variety of surface forms, such as wells, trenches, pins, channels and pores, to which the nucleic acid probes are bound. The shape of the surface is not critical. It can, for example, be a flat surface such as a square, rectangle, or circle; a curved surface; or a three dimensional surface such as a bead, particle, strand, precipitate, tube, sphere, etc. Microfluidic devises are also encompassed by the invention.

In a preferred embodiment, a Composition of nucleic acids is in the form of a microarray (sometimes referred to as a DNA “chip”). Microarrays allow for massively parallel gene expression analysis. See, e.g., Lockhart et al(2002), Nature 405, 827-836 and Phimister (1999), Nature Genetics 21 (supp), 1-60. In a microarray, the array elements are arranged so that there are preferably at least one or more different array elements, more preferably at least about 100 array elements, and most preferably at least about 1,000 array elements, on a 1 cm² substrate surface. The maximum number of array elements is unlimited, and can be at least 100,000 array elements. Furthermore, the hybridization signal from each of the array elements is individually distinguishable.

Methods of making DNA arrays, including microarrays are conventional. For example, the probes may be synthesized directly on the surface; or preformed molecules, such as oligonucleotides or cDNAs, may be introduced onto (e.g., bound to, or otherwise immobilized on) the surface. Among suitable fabrication methods are photolithography, pipetting, drop-touch, piezoelectric printing (ink-j et), or the like. For some typical methods, see Ekins et al. (1999), Trends in Biotech 17, 217-218; Healey et al. (1995) Science 269, 1078-80; WO95/251116; WO95/35505; and U.S. Pat. No. 5,605,662.

Furthermore, the probes do not have to be directly bound to the substrate, but rather can be bound to the substrate through a linker group. The linker groups are typically about 6 to 50 atoms long to provide exposure to the attached nucleic acid probe. Preferred linker groups include ethylene glycol oligomers, diamines, diacids and the like. Reactive groups on the substrate surface react with one of the terminal portions of the linker to bind the linker to the substrate. The other terminal portion of the linker is then functionalized for binding the nucleic acid probe.

A composition of the invention may comprise, optionally, nucleic acids (or polypeptides, or antibodies) that act as internal controls. The controls may be positive controls or negative controls, examples of which will be evident to the skilled worker.

Another aspect of the invention is a composition (combination) comprising at least two isolated polypeptides that are of a size and structure that can be recognized by, and/or bound by, an antibody. That is, the polypeptides are antigenic. The polypeptides can be selected from polypeptides that correspond to the genes noted above (e.g., genes 1-15 from Table 5, genes 1-30 from Table 7, or the additional genes listed in Tables 3, 4 or 6). The composition may contain polypeptides corresponding to any combination of two or more of the genes of the invention. In a composition of the invention, the total number of isolated polypeptides in the composition is generally no more than about 9,000 (e.g. no more than about 5,000; 1,000; 500; 150; 75; 50), although larger numbers can be used.

Specifically, the composition may comprise one or a plurality of isolated antigenic polypeptides selected from polypeptides that correspond to the combinations of genes noted above with respect to nucleic acid compositions. For example, the compositions may comprise polypeptides selected from:

(a) polypeptides comprising SEQ ID NOs: 17-32 and/or SEQ ID NOs: 59-84;

(b) polypeptides encoded by polynucleotides comprising SEQ ID NOs: 1-16 and/or 33-58;

(c) polypeptides whose sequences are at least about 85% (e.g., at least about 90%, 95%, or 98%) identical to SEQ ID NOs: 17-32 and/or SEQ ID NOs: 59-84;

(d) antigenic fragments of (a), (b) or (c); and/or

(e) active variants of (a), (b), (c) or (d);

wherein the polypeptides, active variants or antigenic fragments are of a size and structure that can be recognized, or bound by, an antibody.

An “active” variant or fragment of a polypeptide of the invention is one which is able to bind to, or to elicit, an antibody that is specific for the polypeptide. For example, polypeptides comprising small substitutions, additions, deletions, etc, are tolerated provided they retain the ability to elicit a desired antibody, as are suitable antigenic fragments of the polypeptides. Antigens that exhibit at least about 90% (e.g., at least about 95%, or at least about 98%) sequence identity to a polypeptide of the invention, or to a fragment thereof, are also tolerated. Methods for determining if a polypeptide exhibits a particular percent identity to a polypeptide of the invention are conventional; algorithms such as those discussed elsewhere herein in regard to nucleic acids can be used. A composition of the invention may contain more than one active polypeptide fragment corresponding to a gene of the invention.

One use of such compositions of polypeptides of the invention is as a source for generating antibodies that can be used to help diagnose CIDP or vasculitic neuropathy. One embodiment is a composition comprising one or a plurality of (e.g., at least about 5, 10 or 15) isolated, antigenic, polypeptides for use in generating antibodies for detecting the expression of genes associated with CIDP or vasculitic neuropathy.

A composition of polypeptides of the invention may comprise any combination of, e.g., at least about 1, 2, 5, 10, 15, 25, 50, 55, 60, 75, 100 or more of the mentioned isolated polypeptides, variants or fragments that correspond to genes from Tables 3-7. A polypeptide composition of the invention may comprise, consist essentially of, or consist of, e.g., at least about 1, 2, 5, 10, 15, 25, 50, 75, 100, 200, 500, 750, 1,000, 2,000, 3,000, 5,000, 10,000, 25,000, 50,000, 100,000, 200,000, 500,000, 1×10⁶, 5×10⁶ or more total isolated polypeptides.

Another aspect of the invention is a composition of antibodies which are specific for, and/or generated from, the polypeptides of the invention. As used herein, an antibody that is “specific for” a polypeptide includes an antibody that binds selectively to the polypeptide and not generally to other polypeptides unintended for binding to the antibody. The parameters required to achieve such specificity can be determined routinely, using conventional methods in the art. The antibodies may be specific for polypeptides comprising SEQ ID NOs: 17-32, 59-84, and/or sequences of the polypeptides listed in Tables 3, 4 and 6, or for active variants or fragments of these polypeptides.

One embodiment of the invention is a composition comprising selected numbers of such antibodies, which are in a form that permits their binding to the polypeptides for which they are specific. Specifically, the composition may comprise one or a plurality of isolated antibodies (preferably at least about 5, 10 or 15 isolated antibodies), which are selected from antibodies that are specific for polypeptides corresponding to the genes from Tables 3-7. The composition may contain antibodies which are specific for polypeptides corresponding to any combination of two or more genes of the invention. For example, the antibodies may be specific for polypeptides selected from:

(a) polypeptides comprising SEQ ID NOs: 17-32 and/or SEQ ID NOs: 59-84;

(b) polypeptides encoded by polynucleotides comprising SEQ ID NOs: 1-16 and/or 33-58;

(c) polypeptides whose sequences are at least about 85% (e.g., at least about 90%, 95%, or 98%) identical to SEQ ID NOs: 17-32 and/or SEQ ID NOs: 59-84;

(d) antigenic fragments of (a), (b) or (c); and/or

(e) active variants of (a), (b), (c) or (d);

wherein the polypeptides, active variants or antigenic fragments are of a size and structure that can be recognized, or bound by, an antibody.

Generally, the antigenic fragments comprise at least about 8 or at least about 12 contiguous amino acids of said polypeptide sequences.

The antibody compositions of the invention may be used, e.g., to detect the expression of genes associated with CIDP or vasculitic neuropathy.

The above compositions may comprise any combination of, e.g., at least about 1, 2, 5, 10, 15, 20, 25, 35, 45, 55, 65, 75, 100, 200, 300, 400, 500 or more of the mentioned isolated antibodies or antibody fragments specific for genes of the invention. An antibody composition of the invention may comprise, consist essentially of, or consist of a total of, e.g., at least about 1, 2, 5, 10, 15, 20, 25, 50, 60, 70, 100, 125, 150, 200, 250, 300, 400, 500, 750, 1,000, 2,000, 3,000, 5,000, 7,000; 8,000; 9,000; 10,000, 11,000; 12,000; 13,000; 14,000; 15,000; 25,000, 50,000, 100,000, 200,000, 500,000, 1×10⁶ or more isolated antibodies. In embodiments of the invention, the composition comprises no more than about 1,000 (e.g., no more than about 500,000; 200,000; 100,000; 50,000; 25,000; 14,000; 13,000; 12,000; 11,000; 10,000; 9,000; 8,000; 7,000; 6,000; 5,000, 4,000; 3,000; 2,000; 1,000; 750; 500; 400; 300; 250; 200; 150; 125; 100; 70; 60; 50; 25; 20; 15; 10; 5; 2; or 1) total isolated antibodies.

The isolated antibodies in any of the above compositions may be in the form of an aqueous solution (e.g., in a form suitable for radioimmunoassay), or the isolated antibodies may be immobilized on a substrate. In embodiments of the invention, the isolated antibodies are in an array, e.g., a microarray; they may be reactive elements on an array, such as a microarray. By “reactive” elements is meant that the antibodies can react, e.g., bind, in a specific manner, with antigens for which they are specific.

In one embodiment, antibodies of the invention are immobilized on a surface (e.g., are reactive elements on an array, such as a microarray, or are on another surface, such as used for surface plasmon resonance (SPR)-based technology, such as Biacore), and polypeptides in the sample are detected by virtue of their ability to bind specifically to the antibodies. Methods of preparing the surfaces and performing the analyses are conventional.

Any of a variety of antibodies can be used in methods of the invention. Such antibodies include, e.g., polyclonal, monoclonal (mAbs), recombinant, humanized or partially humanized, single chain, Fab, and fragments thereof. The antibodies can be of any isotype, e.g., IgM, various IgG isotypes such as IgG₁′ IgG₂ₐ, etc., and they can be from any animal species that produces antibodies, including goat, rabbit, mouse, chicken or the like. An antibody “specific for” a polypeptide means that the antibody recognizes a defined sequence of amino acids, or epitope, either present in the full length polypeptide or in a peptide fragment thereof.

Antibodies can be prepared according to conventional method, which are well known, e.g. Green et al., Production of Polyclonal Antisera, in Immunochemical Protocols (Manson, ed.), (Humana Press 1992); Coligan et al., in Current Protocols in Immunology, Sec. 2.4.1 (1992); Kohler & Milstein (1975), Nature 256, 495; Coligan et al., sections 2.5.1-2.6.7; and Harlow et al., Antibodies: A Laboratory Manual, page 726 (Cold Spring Harbor Laboratory Pub. 1988). Methods of preparing humanized or partially humanized antibodies, and antibody fragments, and methods of purifying antibodies, are conventional.

Another aspect of the invention is a method for detecting (e.g., measuring, or quantitating) the expression of genes associated with a peripheral neuropathy in a subject with neuropathy of otherwise unknown etiology, who is suspected of having CIDP or vasculitic neuropathy. The method comprises determining in a sample from the subject (which represents expressed genes (polynucleotides or polypeptides)), the level of expression, compared to a baseline value, of polynucleotides or polypeptides whose expression level is correlated with CIDP or vascultic neuropathy, as discussed above. Any of the compositions of the invention can be used.

In one embodiment, this method involves contacting a sample from the subject, which is a peripheral nerve or which contains peripheral nerve fibers, with a composition of nucleic acids or of antibodies of the invention, under conditions effective for specific binding of the nucleic acids to the polynucleotides in the sample (such as hybridization under conditions of high stringency, or hybridization under conditions effective for a PCR probe of the invention to bind to a target polynucleotide), or effective for specific binding of the antibodies to the polypeptides in the sample. The method may further comprise detecting (e.g., determining the amount of) the polynucleotides in the sample which have bound to the nucleic acids, or detecting (e.g., determining the amount of) the polypeptides in the sample which have bound to the antibodies. In general, amounts of the polynucleotides or polypeptides that are detected reflect the degree of expression (either up-regulation or down-regulation) of genes whose expression is correlated with CIDP or vasculitic neuropathy.

In one embodiment of this method, the expression level is determined, compared to a baseline value, of

(a) one or more of NQO1, NR1D1 and SCD, and

(b) one or more of TAC1 and AIF1.

A significant increase in the degree of expression of one or more of the genes in (a) and of one or more of the genes in (b) indicates that the subject is likely to be suffering from (has a high likelihood of suffering from) CIDP. The absence of a significant degree of over-expression of the gene(s) in (a), and the presence of a significant degree of over-expression of one or more of the genes in (b) indicates that the subject is likely to be suffering from (has a high likelihood of suffering from) vasculitic neuropathy. As the number of marker genes which are over-expressed increases, the likelihood that the subject is suffering from the condition increases.

A “significant” increase or decrease in the expression level, as used herein, means that the value obtained in the test sample is greater than 2 standard deviations above the mean obtained with a group of control samples (p<0.05). A significant decrease in the expression level, as used herein, means that the value in the test sample is less than 2 standard deviations below the mean obtained with controls (p<0.05).

In another embodiments, the set of genes in (b) further comprises one or more of MSR1, PCKS1 and CLCA2. A significant increase in the degree of over-expression of one or more of these three genes indicates a further increased likelihood that the subject is suffering from either CIPD or vasculitic neuropathy.

In another embodiment, the set of genes in (b) further comprises one or more additional genes from Table 5. A significant increase in the degree of expression of the further gene(s) from Table 5 indicates a further increased likelihood that the subject is suffering from CIPD.

In another embodiment, the set of genes in (b) further comprises one or more additional genes from Table 7. A significant increase in the degree of expression of the further gene(s) from Table 7 indicates a further increased likelihood that the subject is suffering from vasculitic neuropathy.

In another embodiment, the set of genes in addition to one or more of NQO1, NR1D1 and SCD further comprises one or more additional genes from Tables 3, 4, 5, 6 and/or 7. A significant increase in the degree of expression of the further gene(s) from Table 3 or Table 5, or a significant decrease in the degree of expression of the further gene(s) from Table 4, indicates a further increased likelihood that the subject is suffering from CIPD. A significant increase in the degree of expression of the further gene(s) from Table 6 or Table 7 (or a decrease with regard to the two final genes in Table 6) indicates a further increased likelihood that the subject is suffering from vasculitic neuropathy.

In assays described herein, a given polynucleotide or polypeptide may or may not be expressed in an increased or decreased amount, compared to a baseline value, in a sample from a given subject. In a general sense, this invention relates to methods to determine if a gene product is expressed in an increased or decreased amount, irrespective of whether such increased or decreased expression is detected.

The baseline value may be obtained, for example, by hybridizing a nucleic acid composition of the invention, under conditions of high stringency, to a control polynucleotide sample. Suitable constitutively expressed genes that can be used as controls are discussed elsewhere herein. In one embodiment, a baseline value is determined by obtaining a polynucleotide sample from normal tissue, as discussed elsewhere herein. Comparable baseline values can be obtained for polypeptide expression, using conventional methods.

In another embodiment of the invention, for determining if a subject has a likelihood of having vasculitic neuropathy, the amount of expression, compared to a baseline value, is determined for one or more of a set of genes comprising:

(a) one, two, three, four or five of genes #1-5 in Table 7; and/or

(b) one, two, three, four or five of genes #6-10 in Table 7; and/or

(c) one, two, three, four or five of genes #11-15 in Table 7; and/or

(d) one, two, three, four or five of genes #16-20 in Table 7; and/or

(e) one, two, three, four or five of genes #21-25 in Table 7; and/or

(f) one, two, three, four or five of genes #25-30 in Table 7.

A significant increase in the degree of expression of the gene(s) indicates an increased likelihood that the subject is suffering from vasculitic neuropathy.

An assay of the invention is generally carried out on a subject (patient) who exhibits symptoms of peripheral neuropathy, but for whom a variety of potential causes of peripheral neuropathy, such as diabetes, hereditary disease, nutritional deficiencies, drugs, toxins, infections, cancer, thyroid disease and renal failure, among others, have been ruled unlikely. That is, the subject has neuropathy of otherwise unknown etiology, but is suspected of having CIDP or vasculitic neuropathy. The subject is a generally a vertebrate, such as a mammal (e.g. agricultural or domestic animal, such as a dog); preferably, the subject is a human patient.

A variety of suitable sample sources can be used. In general, it is preferable to use a peripheral nerve (such as a sural nerve), or a tissue which contains peripheral nerve fibers, such as a skin sample (a punch biopsy). See Example IV for a further discussion of skin biopsies. Any nerve or tissue that is obviously affected by the neuropathy can be used for testing. This includes, e.g., a piece of nerve that innervates a weak muscle or a region in which there is altered, or loss of, sensation. As both vascultic neuropathy and CIDP are diffuse diseases, areas that appear uninvolved may also be subclinically affected. They might still manifest the changes that can be detected by differential gene expression. Thus, any nerve or tissue containing nerves (or nerve fibers) can be used to make a diagnosis.

Vasculitic neuropathy can also occur as part of a generalized or systemic vasculitis, sometimes in association with collagen vascular diseases or hepatitis C infection. Tests for these conditions can provide clues to the diagnosis, but the diagnosis can only be definitively made by pathological studies that show inflammation in the blood vessel walls. As the markers identified herein for vasculitic neuropathy are expected to occur in any tissue that is affected by vasculitis, even in cases where nerves are not affected, the markers identified for vasculitic neuropathy can be useful for the diagnosis of systemic vasculitis, even in the absence of neuropathy, or with subclinical neuropathy. Of course, samples other than nerve-containing samples must be assayed. For example, if other organs are affected, these can be biopsied instead of the nerves, to diagnose vasculitis. Some typical sample sources are discussed in Example V.

In order to conduct an analysis of expressed genes, a sample derived from a subject is manipulated so that it represents expressed genes, either polynucleotides or polypeptides translated from them. As used herein, “polynucleotide” refers to a target whose expression is analyzed, whereas “nucleic acid” refers to a composition (of probes) used to analyze the expression of the polynucleotides.

DNA or RNA can be isolated according to any of a number of methods well known to those of skill in the art. For example, methods of purification of nucleic acids are described in Laboratory Techniques in Biochemistry and Molecular Biology: Hybridization With Nucleic Acid Probes, Part I. Theory and Nucleic Acid Preparation, P. Tijssen, ed. Elsevier, New York, N.Y. (1993). In one case, total RNA is isolated using the TRIZOL total RNA isolation reagent (Life Technologies, Gaithersburg, Md.) and mRNA is isolated using oligo d(T) column chromatography or glass beads. Alternatively, when target polynucleotides are derived from an mRNA, the target polynucleotide can be a cDNA reverse transcribed from an mRNA, an RNA transcribed from that cDNA, a DNA amplified from that cDNA, an RNA transcribed from the amplified DNA, or the like. The Examples herein describe typical methods for amplifying the low levels of mRNA which may be obtained, e.g. from skin samples. Accordingly, a polynucleotide sample “representing expressed genes” can comprise, e.g., mRNA, cRNA, cDNA, PCR products, or the like.

In some embodiments of the invention, e.g. when samples are peripheral nerves, such as sural nerve, samples are amplified using non-specific primers, such as oligo dT/random primer combinations. In another embodiment, it may be desirable to specifically amplify markers of interest, in order to reduce the contribution of expressed genes which are not markers for the disease of interest (e.g. CIDP or vasculitic neuropathy). This may be beneficial, e.g., for the analysis of skin samples. In this embodiment, PCR primers are used which are specific for the genes of interest, e.g., for the genes in Table 5 or Table 7. Two or more genes of interest may be amplified simultaneously. Suitable PCR primers can be selected using routine, art-recognized methods.

Methods for designing PCR primers and for carrying out PCR reactions (e.g. real time PCR), including reaction conditions, such as the presence of salts, buffers, ATP, dNTPs, etc. and the times and temperature of incubation, are conventional and can be optimized readily by one of skill in the art. See, e.g., Innis et al., editors, PCR Protocols (Academic Press, New York, 1990); McPherson et al., editors, PCR: A Practical Approach, Volumes 1 and 2 (IRL Press, Oxford, 1991, 1995); Barany (1991) PCR Methods and Applications 1, 5-16; Diffenbach et al., editors, PCR Primers, A Laboratory Manual (Cold Spring Harbor Press); etc.

It is advantageous to include quantitation controls within the sample to assure that amplification and labeling procedures do not change the true distribution of target polynucleotides in a sample. For this purpose, a sample can be spiked with a known amount of a control target polynucleotide and the composition of nucleic acid probes can include reference nucleic acid probes which specifically hybridize with the control target polynucleotides. As used herein, the singular forms “a,” “an,” and “the” include plural referents unless the context clearly dictates otherwise. For example, “a” control target, as used above, includes two or more control targets. After hybridization and processing, the hybridization signals obtained should reflect accurately the amounts of control target polynucleotide added to the sample.

In one embodiment of the method, the amount (level of expression) of polynucleotides in a sample is determined by hybridizing polynucleotides in the sample to a nucleic acid composition of the invention, under conditions of high stringency, and comparing the amount of hybridization to a baseline value. In embodiments of this method, the nucleic acids are immobilized on a substrate, and/or are in an array, e.g. are hybridizable elements on an array, such as a microarray.

The amount of hybridization of a polynucleotide in the sample to a nucleic acid specific for it in the nucleic acid composition generally reflects the level of expression of the polynucleotide in the sample.

Hybridization causes a denatured nucleic acid probe and a denatured complementary target polynucleotide to form a stable duplex through base pairing. Hybridization methods are well known to those skilled in the art (See, for example, Laboratory Techniques in Biochemistry and Molecular Biology, Vol. 24: Hybridization With Nucleic Acid Probes, P. Tijssen, ed. Elsevier, New York, N.Y. (1993)). Conditions can be selected for hybridization where exactly complementary target and nucleic acid probe can hybridize, i.e., each base pair must interact with its complementary base pair. Alternatively, conditions can be selected where target and probes have mismatches but are still able to hybridize. Suitable conditions can be selected, for example, by varying the concentrations of salt or formamide in the prehybridization, hybridization and wash solutions, or by varying the hybridization and wash temperatures.

Hybridization can be performed at low stringency with buffers, such as 6×SSPE with 0.005% Triton X-100 at 37° C., which permits hybridization between target and polynucleotide probes that contain some mismatches to form target polynucleotide/probe complexes. Subsequent washes are performed at higher stringency with buffers, such as 0.5×SSPE with 0.005% Triton X-100 at 50° C., to retain hybridization of only those target/probe complexes that contain exactly complementary sequences. Alternatively, hybridization can be performed with buffers, such as 5×SSC/0.2% SDS at 60° C., and washes performed in 2×SSC/0.2% SDS and then in 0.1×SSC. Stringency can also be increased by adding agents such as formamide. Background signals can be reduced by the use of detergent, such as sodium dodecyl sulfate, Sarcosyl or Triton X-100, or a blocking agent, such as sperm DNA or bovine serum albumin (BSA).

In a preferred embodiment, nucleic acid probes of the invention hybridize specifically to target polynucleotides of interest under conditions of high stringency. As used herein, “conditions of high stringency” or “high stringent hybridization conditions” means any conditions in which hybridization will occur when there is at least about 95%, preferably about 97 to 100%, nucleotide complementarity (identity) between the nucleic acids (e.g., a polynucleotide of interest and a nucleic acid probe). Generally, high stringency conditions are selected to be about 5° C. to 20° C. lower than the thermal melting point (Tₘ) for the specific sequence at a defined ionic strength and pH. Appropriate high stringent hybridization conditions include, e.g., hybridization in a buffer such as, for example, 6×SSPE-T (0.9 M NaCl, 60 mM NaH₂ PO₄, 6 mM EDTA and 0.05% Triton X-100) for between about 10 minutes and about at least 3 hours (in a preferred embodiment, at least about 15 minutes) at a temperature ranging from about 4° C. to about 37° C.). In one embodiment, hybridization under high stringent conditions is carried out in 5×SSC, 50% deionized Formamide, 0.1% SDS at 42° C. overnight. used to help confirm, or increase the likelihood, that the subject has CIDP.

Hybridization specificity can be evaluated by comparing the hybridization of specificity-control nucleic acid probes to specificity-control target polynucleotides that are added to a sample in a known amount. The specificity-control target polynucleotides may have one or more sequence mismatches compared with the corresponding nucleic acid probes. In this manner, whether only complementary target polynucleotides are hybridizing to the nucleic acid probes or whether mismatched hybrid duplexes are forming is determined.

Hybridization reactions can be performed in absolute or differential hybridization formats. In the absolute hybridization format, target polynucleotides from one sample are hybridized to the probes in an array (e.g., in a microarray format) and signals detected after hybridization complex formation correlate to target polynucleotide levels in a sample. In the differential hybridization format, the differential expression of a set of genes in two biological samples is analyzed. For differential hybridization, target polynucleotides from both biological samples are prepared and labeled with different labeling moieties. A mixture of the two labeled target polynucleotides is added to an array (e.g., a microarray). The array is then examined under conditions in which the emissions from the two different labels are individually detectable. Probes in the array that are hybridized to substantially equal numbers of target polynucleotides derived from both biological samples give a distinct combined fluorescence (Shalon et al. PCT publication WO95/35505). In one embodiment, the labels are fluorescent labels with distinguishable emission spectra, such as a lissamine conjugated nucleotide analog and a fluorescein conjugated nucleotide-analog. In another embodiment Cy3/Cy5 fluorophores (Amersham Pharmacia Biotech) are employed.

After hybridization, the array (e.g., microarray) is washed to remove nonhybridized polynucleotides and complex formation between the hybridizable array elements and the target polynucleotides is detected. Methods for detecting complex formation are well known to those skilled in the art. In a preferred embodiment, the target polynucleotides are labeled with a fluorescent label and levels and patterns of fluorescence indicative of complex formation are measured. In one embodiment, the measurement is accomplished by fluorescence microscopy, e.g., confocal fluorescence microscopy. An argon ion laser excites the fluorescent label, emissions are directed to a photomultiplier and the amount of emitted light detected and quantitated. The detected signal should be proportional to the amount of probe/target polynucleotide complex at each position of the microarray. The fluorescence microscope can be associated with a computer-driven scanner device to generate a quantitative two-dimensional image of hybridization intensity. The scanned image is examined to determine the abundance/expression level of each hybridized target polynucleotide. In another embodiment, the measurement of levels and patterns of fluorescence is accomplished with a fluorescent imaging device, such as a microarray scanner (e.g., Axon scanner with GenePix Pro software). As with the previous measurement method, the measurements can be used to determine the abundance/expression level of each hybridized target polynucleotide.

In a differential hybridization experiment, target polynucleotides from two or more different biological samples are labeled with two or more different fluorescent labels with different emission wavelengths. Fluorescent signals are detected separately with different photomultipliers set to detect specific wavelengths. The relative abundances/expression levels of the target polynucleotides in two or more samples is obtained.

Typically, array fluorescence intensities can be normalized to take into account variations in hybridization intensities when more than one array is used under similar test conditions. In a preferred embodiment, individual probe/target complex hybridization intensities are normalized using the intensities derived from internal normalization controls contained on each microarray.

It may be desirable to fragment the target polynucleotides prior to hybridization. Fragmentation improves hybridization by minimizing secondary structure and cross-hybridization to other nucleic acid target polynucleotides in the sample or noncomplementary nucleic acid probes. Fragmentation can be performed by mechanical, enzymatic or chemical means.

The target polynucleotides may be labeled with one or more labeling moieties to allow for detection of hybridized probe/target polynucleotide complexes. The labeling moieties can include compositions that can be detected by spectroscopic, photochemical, biochemical, bioelectronic, immunochemical, electrical, optical or chemical means. The labeling moieties include radioisotopes, such as ³²P, ³³P or ³⁵S, chemiluminescent compounds, labeled binding proteins, heavy metal atoms, spectroscopic markers, such as fluorescent markers and dyes, magnetic labels, linked enzymes, mass spectrometry tags, spin labels, electron transfer donors and acceptors, and the like. In one embodiment, a fluorescent dye is incorporated directly by using a fluorochrome conjugated nucleotide triphosphate (e.g. Cy3-dUTP) or through a secondary coupling reaction by first incorporating an amino allyl conjugated nucleotide triphosphate (e.g. amino allyl-dUTP) followed by chemical coupling of the fluorochrome (e.g. NHS-Cy3).

Exemplary dyes include quinoline dyes, triarylmethane dyes, phthaleins, azo dyes, cyanine dyes and the like. Preferably, fluorescent markers absorb light above about 300 nm, preferably above 400 nm, and usually emit light at wavelengths at least greater than 10 nm above the wavelength of the light absorbed. Specific preferred fluorescent markers include fluorescein, phycoerythrin, rhodamine, lissamine, and Cy3 and Cy5 available from Amersham Pharmacia Biotech (Piscataway, N.J.).

Labeling can be carried out during an amplification reaction, such as polymerase chain and in vitro transcription reactions, or by nick translation or 5′ or 3′-end-labeling reactions. In one embodiment, labeled nucleotides are used in an in vitro transcription reaction. When the label is incorporated after or without an amplification step, the label is incorporated by using terminal transferase or by kinasing the 5′ end of the target polynucleotide and then incubating overnight with a labeled oligonucleotide in the presence of T4 RNA ligase.

Alternatively, the labeling moiety can be incorporated after hybridization once a probe/target complex has formed. In one case, biotin is first incorporated during an amplification step as described above. After the hybridization reaction, unbound polynucleotides are rinsed away so that the only biotin remaining bound to the substrate is that attached to target polynucleotides that are hybridized to the nucleic acid probes. Then, an avidin-conjugated fluorophore, such as avidin-phycoerythrin, that binds with high affinity to biotin is added. In another case, the labeling moiety is incorporated by intercalation into preformed target/polynucleotide probe complexes. In this case, an intercalating dye such as a psoralen-linked dye can be employed.

In another embodiment of this method, the determination of the amount (level of expression) of polynucleotides in a sample is performed by quantitatively amplifying polynucleotides in the sample with primers specific for those polynucleotides, and comparing the amount of amplified polynucleotide to a baseline value. For example, conventional methods of RT-PCR may be used. Methods for selecting suitable amplification primers, based on the sequences disclosed herein, for optimizing amplification conditions, and for detecting and quantitating the amplified product, are conventional. Some such procedures are discussed herein with reference to amplifying nucleic acid samples in preparation for hybridization assays. One method for quantitating the amount of expressed nucleic acid is real time RT-PCR. Methods for performing this assay are conventional. Generally, detectable labels are attached to reporter probes. Fluorophore-containing TacMan™ probes can be used. See, e.g., the “TaqMan PCR” (PE Applied Biosystems) manual; Livak et al. (1995) PCR Methods and Applications 4, 357-362, or the like. Also, see the Examples herein.

In another embodiment, the method comprises determining in a polypeptide sample from a subject the amount (level of expression), compared to the amount (level of expression) of a baseline value, of each of one or a plurality of protein products (polypeptides) whose expression is correlated with CIDP or vasculitic neuropathy. Polypeptides whose expression is measured include those comprising SEQ ID NOs: 17-32, 59-84, and the polypeptides in Tables 3, 4 and 6, whose sequences can be obtained from the GenBank reference numbers in those Tables. The presence or quantity of the protein product in a sample from the subject, is determined, and compared to a baseline value.

Methods of preparing samples (e.g., from patients) for polypeptide analysis are conventional and well-known in the art, and a variety of methods known to skilled workers can be used to determine the amount of these proteins. For example, enzymatic activities of the proteins can be measured, using conventional procedures. Alternatively, the proteins can be detected by immunological methods such as, e.g., immunoassays (EIA), radioimmunoassay (RIA), immunofluorescence microscopy, or immunohistochemistry, all of which assay methods are fully conventional. See, e.g., U.S. Pat. No. 6,602,661.

In one embodiment of this method, the determination is performed by:

contacting the polypeptide sample with an antibody composition containing one or a plurality of antibodies specific for polypeptides as described above, under conditions effective for at least one of said antibodies to bind specifically to the corresponding polypeptide (polypeptide for which it is specific), and

comparing the amount (degree) of specific binding of to a baseline value. The amount of binding of a polypeptide in the sample to an antibody specific for it in the antibody composition generally reflects the amount (level of expression) of the polypeptide in the sample. The baseline value may reflect the amount of the polypeptides expressed in normal tissue. For example, it may be obtained by contacting the antibody composition, under conditions as above, to a polypeptide sample obtained from normal tissue, as described above.

The antibody composition may be in the form of an aqueous solution; the antibodies may be immobilized on a substrate or surface (e.g., a surface suitable for surface plasmon resonance (SPR)-based technology); and/or the antibodies may be in an array, e.g. they may be reactive elements on an array, such as a microarray.

Other aspects of the invention are kits suitable for performing any of the methods of the invention.

One embodiment of the invention is a kit (e.g. for detecting the presence and/or amount of a polynucleotide in a sample from a subject having, or suspected of having, a peripheral neuropathy (e.g. CIPD or vasculitic neuropathy). The kit can comprise a composition of nucleic acids of the invention (e.g., in the form of an array, such as a microarray) and, optionally, one or more reagents that facilitate hybridization of the nucleic acids in the composition to a test polynucleotide of interest, and/or that facilitate detection of the hybridized polynucleotide(s), e.g., that facilitate detection of fluorescence. The kit may comprise a composition of nucleic acids of the invention (e.g., in the form of an array), means for carrying out hybridization of the nucleic acids in the array to a test polynucleotide(s) of interest, and/or means for reading hybridization results. Hybridization results may be units of fluorescence.

Another embodiment is a kit for detecting the presence and/or amount of a polypeptide in a sample from a subject having, or suspected of having, a peripheral neuropathy (e.g. CIPD or vasculitic neuropathy), comprising a composition of antibodies of the invention (e.g., in the form of an array) and, optionally, one or more reagents that facilitate binding of the antibodies in the composition with a test protein(s) of interest, or that facilitate detection of bound antibody. The kit may comprise a composition of antibodies of the invention (e.g., in the form of an array or a Biacore chip), means for carrying out binding of the antibodies in the array to a test polypeptide(s) of interest, and means for reading the binding results.

Kits of the invention may comprise instructions for performing a method, such as a diagnostic method. Other optional elements of a kit of the invention include suitable buffers, media components, or the like; a computer or computer-readable medium for storing and/or evaluating the assay results; containers; or packaging materials. Reagents for performing suitable controls may also be included. The reagents of the kit may be in containers in which the reagents are stable, e.g., in lyophilized form or stabilized liquids. The reagents may also be in single use form, e.g., in single reaction form for diagnostic use.

The present invention also relates to combinations of the invention in which the nucleic acid or protein sequences of the invention are represented, not by physical molecules, but by computer-implemented databases. For example, the present invention relates to electronic forms of polynucleotides, polypeptides, antibodies, etc., of the present invention, including a computer-readable medium (e.g., magnetic, optical, etc., stored in any suitable format, such as flat files or hierarchical files) which comprise such sequences, or fragments thereof, e-commerce-related means, etc. An investigator may, e.g., compare an expression profile exhibited by a sample from a subject to an electronic form of one of the expression profiles of the invention, and may thereby diagnose whether the subject is suffering from a particular form of peripheral neuropathy (e.g., CIPD or vasculitic neuropathy).

Having now generally described the invention, the same will be more readily understood through reference to the following examples which are provided by way of illustration, and are not intended to be limiting of the present invention, unless specified. In the foregoing and in the following examples, all temperatures are set forth uncorrected in degrees Celsius; and, unless otherwise indicated, all parts and percentages are by weight.

### EXAMPLES

### Example I

### Patients and Methods

A. Patients

Nerve biopsies from eight patients with CIDP were included in the study. The diagnosis was based on clinical, pathological and electrophysiological criteria (Berger et al. (2003), supra). The characteristics of the CIDP patients and nerve biopsies are listed in Table 1. In addition, nerve biopsies of three patients with vasculitis representing an inflammatory nondemyelinating pathology were included; patients were diagnosed using conventional procedures. As normal controls, biopsy specimens were obtained from three individuals who did not suffer from polyneuropathy but from myopathy, muscular dystrophy and dermatomyositis, respectively.

TABLE 1
CIDP patient data

| | Age | | Biopsy time | | | | | |
|---|---|---|---|---|---|---|---|---|
| Patient | (years) | Sex | after onset | M/S | Course | CSF | EMG | Pathology |
| 1 | 49 | F | 72 months | S > M | RR | n.a. | Sensorimotor | Segmental demyelination |
| | | | | | | | demyelinating | and remyelination no |
| | | | | | | | | infiltrates. Muscle: mild |
| | | | | | | | | neurogenic abnormalities |
| 2 | 54 | M | Several years | S > M | Progressive | n.d. | Absent SNAP, | Severe loss of large- |
| | | | | | | | normal motor | diameter myelinated |
| | | | | | | | nerve conduction | fibers, segmental |
| | | | | | | | and myography | remyelination, no |
| | | | | | | | | infiltrates. |
| | | | | | | | | Muscle: mild neurogenic |
| | | | | | | | | abnormalities |
| 3 | 20 | M | 20 months | S > M | Progressive | 2 cells, | Absent SNAPs, | Loss of mainly large |
| | | | | | | 85% lymphocytes | normal motor | myelinated fibers, |
| | | | | | | TP 30 mg/dl | nerve conduction | thinning of myelin |
| | | | | | | | | lamellae, perinodal |
| | | | | | | | | demyelination, no |
| | | | | | | | | infiltrates. |
| | | | | | | | | Muscle: reinnervation |
| 4 | 47 | M | 22 months | S > M | RR | TP 52 mg/dl | Sensorimotor, | Mild loss of myelinated |
| | | | | | | | mixed axonal and | fibers, signs of segmental |
| | | | | | | | demyelinating | remyelination no |
| | | | | | | | neuropathy | infiltrates |
| | | | | | | | | Muscle: mild neurogenic |
| | | | | | | | | abnormalities. |
| 5 | 70 | F | 48 months | S > M | RR | n.d. | Normal | Segmental Demyelination |
| | | | | | | | | Muscle: mild neurogenic |
| | | | | | | | | abnormalities. |
| 6 | 39 | F | 5 years | S = M | RR | TP elevated | Sensorimotor | Apparent myelin loss and |
| | | | | | | | demyelinating | interstitial fibrosis, mild |
| | | | | | | | | inflammation(on imuran) |
| 7 | 45 | M | 9 months | S = M | RR | TP 50 mg/dl | Sensorimotor | No pathology (on |
| | | | | | | | demyelinating | prednisone) |
| 8 | 33 | F | 2 years | S = M | RR | n.a. | Sensorimotor | No pathology (on |
| | | | | | | | demyelinating | prednisone) |
| | | | | | | | with partial | |
| | | | | | | | conduction block | |

B. RNA Sample Processing

Human sural nerve biopsies were immediately embedded in the embedding medium Tissue-Tek (Sakura Finetek, USA) and stored at −70° C. The embedded tissue, with each tissue sample weighing ca. 50 mg, was cut with a cryostat (Leitz, Cryostat) in 10 μm sections. Further tissue homogenization was obtained with an electric rotor stator tissue homogenizer (Polytron, Kinematica, Switzerland). For total RNA extraction we used TRIzol reagent (Invitrogen, Carlsbad, Calif.), according to the manufacturers protocol, followed by Rneasy clean-up (Qiagen, Chatsworth, Calif.), a procedure giving a yield of 1 μg per 100 mg of biopsy tissue. RNA yields were measured by UV absorbance and RNA quality was assessed by agarose gel electrophoresis with SYBR® Gold nucleic acid stain (Molecular Probes, Eugene, Oreg.), for visualization of ribosomal RNA band integrity.

C. cRNA Amplification

In general, the standard RNA processing and hybridization protocols as recommended by Affymetrix (Santa Clara, Calif.) were followed in this study; these protocols are available in the Genechip® Expression Analysis Technical Manual. Yields of total RNA for sural nerve biopsy samples were generally low and for the majority of patients it was not possible to use the standard amount of total RNA (>5 μg) as recommended in the standard protocol. Therefore a double linear amplification approach (Eberwine et al. (1992) Proc. Natl. Acad. Sci. USA 89, 3010-3014) was used in the generation of cRNA for hybridization. In these experiments, equal amounts of starting material were used from each patient. 100 ng of total RNA was converted into biotin-labeled cRNA (complementary RNA) using the Gene Chip Eukaryotic Small Sample Target Labeling Assay Version II (Technical Notes No. 701265 Rev. 2, Affymetrix, Santa Clara, Calif.). Double stranded cDNA was created by using the Super Script Double-Stranded cDNA Synthesis Kit (Invitrogen, Carlsbad, Calif.) using the T7-(dT)₂₄-primer [sequence 5′-GGCCAGTGAATTGTAATACGACTCACTATAGGGAGGCGG-(dT)₂₄-3′] (SEQ ID NO:85) (Affymetrix, Santa Clara, Calif.). The cDNA was purified by ethanol precipitation and then used for in vitro transcription using the Ambion MEGAscript T7 Kit (Ambion, Houston, Tex.). The cRNA was then cleaned using the Qiagen Rneasy Mini Kit (Qiagen, Chatsworth, Calif.). In a second cycle the cRNA obtained in the first cycle, was used as a template to create double stranded cDNA using random primers and the Super Script Double-Stranded cDNA Synthesis Kit (Invitrogen, Carlsbad, Calif.). This second round of cDNA synthesis was similar to the first round except that random hexamers were used in priming of first-strand synthesis, with T7-(dT)₂₄ oligomer priming the second strand. The cDNA was cleaned by ethanol precipitation and then used for in vitro transcription using the ENZO BioArray RNA transcript labeling kit (Affymetrix, Santa Clara, Calif.). Biotin-labeled cRNA was purified by Rneasy Kit (Qiagen, Chatsworth, Calif.) and chemically fragmented randomly to approximately 200 bp (200 mM Tris-acetate, pH 8.2, 500 mM KOAc, 150 mM MgOAc) according to the Affymetrix protocol.

D. Expression Profiling

Each fragmented cRNA sample was hybridized to Affymetrix human U133 microarray set for 16 hours at 60 rpm at 45° C. The microarray was washed and stained on the Affymetrix Fluidics Station using instructions and reagents provided by Affymetrix. This involved removal of nonhybridized material and then incubation with phycoerythrin-streptavidin to detect bound cRNA. The signal intensity was amplified by second staining with biotin-labeled antistreptavidin antibody followed by phycoerythrin-streptavidin staining. Fluorescent images were read using the Hewlett-Packard G2500A Gene Array Scanner. The microarrays were processed on the fluidics station under the control of the Microarray Suite software and read.

E. Data Analysis

Affymetrix GeneChip 5.0 was used as the image acquisition software for the U133 chips. The signal, which represents the intensity of each gene, was extracted from the image. The target intensity value from each chip was scaled to 250. Data normalization, log transformation, filtering of genes that were not detected in any of the samples, statistical analysis and pattern study were performed GeneSpring™ v 6.1 software (Silicon Genetics, Redwood City, Calif.).

Array data were globally normalized by using GeneSpring software. Firstly, all of the measurements on each chip were divided by the 50^{th} percentile value (per-chip normalization). Secondly, each gene was normalized to the median value of the samples (per-gene normalization).

Statistical comparison between the different disease types and normal controls was performed using Welch t-test with log transformed data. The cut-off for p-value was set at 0.05. A two-way hierarchical clustering by distance measure was used to group genes that were differentially expressed between the different disease groups and normal controls.

F. Real Time Quantitative Reverse Transcription Polymerase Chain Reaction (RT-PCR)

Real Time quantitative RT-PCR was used to verify the microarray results. Since the yield of total RNA was very low, we used the amplified biotinylated cRNA as starting material. cRNA samples (10 μg) were reverse transcribed to yield first strand cDNA using the Applied Biosystems Reverse Transcription Reagents protocol (Applied Biosystems, Foster City, Calif., USA). The reverse transcription reactions were then diluted 1:10 in distilled H₂O. Taqman assay PCR reactions (Perkin-Elmer-Applied Biosystems) were performed in 96-well optical plates and run in an ABI PRISM® 7700 Sequence Detection System machine. We used the Assay-on-Demand™ Gene Expression Products (Applied Biosystems). For individual reactions, 2.5 μl of each sample were combined with 12.5 μl of 2× Taqman Universal Master Mix, 1.25 μl of Target Assay Mix and 8.75 μl H₂O. Data were extracted and amplification plots generated with ABI SDS software. All amplifications were done in triplicate and threshold cycle (Cₜ) scores were averaged for subsequent calculations of relative expression values. The Cₜ scores represent the cycle number at which fluorescence signal crosses an arbitrary (user-defined) threshold. The Cₜ scores for genes of interest for each sample were normalized against Cₜ scores for the corresponding endogenous control gene, which was GAPDH. Relative expression for disease versus normal controls was determinded by the following calculation, as described in the Applied Biosystems users bulletin on relative Quantitation of Gene Expression and as published (Schmittgen et al. (2000) Anal. Biochem. 285, 194-204):
Relative Expression=2^{-ΔΔCt}
where ΔΔCₜ=(Cₜ disease-Cₜ GAPDH)−(Cₜ normal-Cₜ GAPDH).

For each disease group the mean of relative expression for each sample was calculated.

### Example II

### Results of Gene Profiling Studies

A. Sample and Chip Quality

The yield of RNA varied from 100 ng to 2.9 μg per sample. The integrity of the RNA as seen by SYBR-Gold® staining after gel electrophoresis was intact and the ratio A260/280 as measure of the RNA purity on UV absorbance ranged for most of the samples from 1.79 to 2.06. Only 2 out of 14 samples had a lower A260/280 ratio which is probably due to the older age of the biopsy samples. The Chip quality was good with present calls between 30.5% to 60%. We also looked at the probe sets of specific maintenance genes (GAPDH, beta-actin) that are designed to the 3′, middle, and 5′ regions of the transcript and compared the 3′ probe set signal intensity to the 5′ probe set signal intensity (3′/5′ ratio) as a measure for RNA degradation and efficiency of transcription reaction. The 3′/5′ ratio for beta-actin was in most samples below 20 and only in the same 2 out of the 14 samples higher with 29.03 and 28.02 respectively. We also calculated the 3′/Middle probe set ratio (3′/M) of the beta-actin gene because the M probes lie approximately 430-770 bases from the most 3′ end and may be a more realistic representation of reliability of the array data for those two samples. The resulting 3′/M ratios for the beta-actin gene were with 4.6 and 6.7 acceptable and therefore we decided to use those two samples (Table 2).

TABLE 2
Chip Quality

| | | | 3′5′ ratio | 3′5′ratio Beta |
|---|---|---|---|---|
| Patients | Disease | Present Calls | GAPDH | Actin (3′M′ ratio) |
| 1 | CIDP | 60% | 2.19 | 7.05 |
| 2 | CIDP | 56.2% | 2.26 | 7.43 |
| 3 | CIDP | 51.3% | 2.21 | 18.35 |
| 4 | CIDP | 50% | 3.95 | 19.63 |
| 5 | CIDP | 57.3% | 3.0 | 7.91 |
| 6 | CIDP | 55.5% | 1.84 | 13.13 |
| 7 | CIDP | 51.9% | 3.27 | 29.03 (4.6) |
| 8 | CIDP | 52.6% | 6.8 | 28.02 (6.7) |
| 9 | NN | 55.40% | 2.37 | 10.66 |
| 10 | NN | 42.3% | 3.28 | 3.24 |
| 11 | NN | 52.8% | 1.97 | 9.52 |
| 12 | VAS | 55.3 | 1.72 | 6.02 |
| 13 | VAS | 30.5% | 6.58 | 17.73 |
| 14 | VAS | 57.9 | 1.6 | 7.47 |

B. Quantitative RT-PCR Validation of Differentially Expressed Genes

A subset of 8 transcripts was chosen for validation by quantitative RT-PCR analysis. The genes IL7 (Interleukin 7), TAC1 (Tachykinin 1), Steaoryl CoA Desaturase (SCD), CD69, Dicarbonyl-L-Xylulose Reductase (DCXR,) Pentraxin 3 (PTXR), Hepcidine (HAMP) and Crystallin alpha B (CRYAB) were chosen based on potential functions of encoded proteins (i.e. remyelination in the case of Steaoryl CoA Desaturase or early B and T cell development in the case of Interleukin 7), or because of the extent of differential regulation between the different nerve biopsy groups.

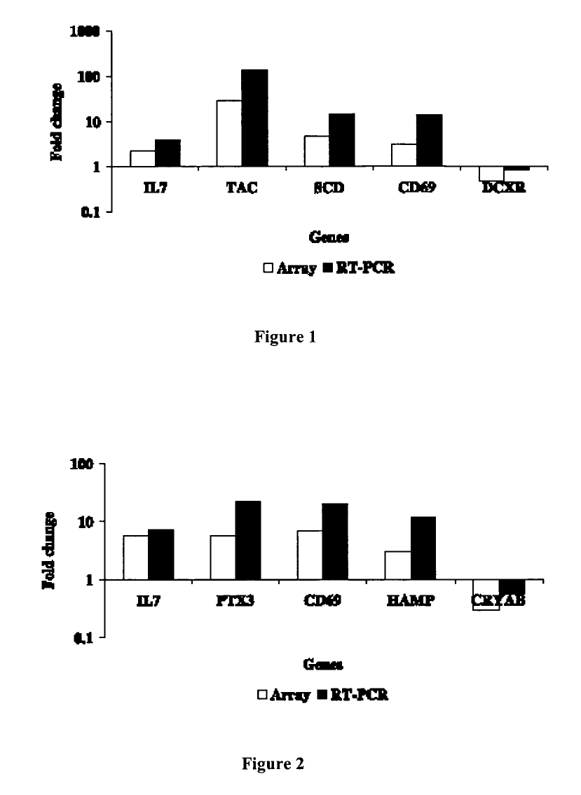

Taqman RT-PCR was used to validate the microarray expression profiling data. The qRT-PCR validation was performed with the amplified biotin labeled cRNA from 7 CIDP, 3NN and 3 VAS biopsies. Data for each gene was normalized to expression of a housekeeping gene, GAPDH. Comparison of RT-PCR and microarray data showed an excellent qualitative agreement (i.e. same trend of induction) (FIGS. 1 and 2).

C. Differentially Regulated Genes

1) CIDP Versus Normal Appearing Nerve

Hierarchical clustering analysis demonstrated distinct gene expression patterns distinguishing CIDP from NN, CIDP from VAS and VAS from NN. In the disease group CIDP versus normal controls, 123 genes were differentially regulated with 101 genes up-regulated and 22 genes down-regulated (greater than twofold change and p<0.05). When we considered only the genes that were present in at least 4 out of 8 CIDP samples for the up-regulated genes and in at least 2 out of 3 control samples for the down-regulated genes 87 genes were differentially regulated. We have listed in Tables 3 and 4 the differentially regulated genes according to their presumed gene ontology. A majority of the differentially expressed genes were involved in signal transduction, metabolism and immunity or inflammation.

TABLE 3
Up-regulated genes in CIDP compared to NN

| | | Fold | | |
|---|---|---|---|---|
| Gene Description | Common Name | change | Affymetrix | GenBank |
| Apoptosis | | | | |
| NCK-associated protein 1 | NAP1, KIAA0587 | 2.566 | 207738—s—at | NM—013436 |
| Cancer | | | | |
| Promyelocytic leukemia | MYL, TRIM19 | 4.615 | 211012_s_at | BC000080 |
| RAB2, member RAS oncogene family | RAB2 | 2.697 | 208733—at | NM—002865 |
| Yamaguchi sarcoma viral related oncogene homolog | LYN | 2.463 | 202625—at | AI356412 |
| V-yes-1 Yamaguchi sarcoma viral related oncogene homolog | JTK8 | 2.357 | 202626—s—at | NM—002350 |
| Cell communication | | | | |
| Placental growth factor, vascular endothelial growth factor-related | PLGF | 3.23 | 209652_s_at | BC001422 |
| protein | | | | |
| Lectin, galactoside-binding, soluble, 2 (galectin 2) | LGALS2 | 2.726 | 208450_at | NM_006498 |
| bone morphogenetic protein | BMP2 | 2.430 | 205289—at | AA583044 |
| Solute carrier family 16 (monocarboxylic acid transporters), | MCT2 | 2.416 | 207057—at | NM—004731 |
| member 7 | | | | |
| Hepatocyte growth factor (hepapoietin A; scatter factor) | HPTA | 2.160 | 210997—at | M77227 |
| Solute carrier family 21 (organic anion transporter), member 9 | OATPB, OATP-B | 2.132 | 203473—at | NM—007256 |
| Integrin, beta 2 (antigen CD18 (p95) | LAD, CD18 | 2.037 | 202803—s—at | NM—000211 |
| Cell cycle regulator | | | | |
| HSPC002 protein. S-phase 2 protein | HSPC002 | 2.130 | 219260—s—at | NM—015362 |
| Enzyme/Metabolism | | | | |
| Stearoyl-CoA desaturase (delta-9-desaturase) | SCD | 16.039 | 211162_x_at | AF116616 |
| Stearoyl-CoA desaturase (delta-9-desaturase) | SCD | 4.660 | 200831—s—at | AA678241 |
| NAD(P)H dehydrogenase, quinone 1 | NQO1 | 3.417 | 201468—s—at | NM—000903 |
| TATA box binding protein (TBP) - associated factor, RNA | TAF1C | 2.447 | 203937_s_at | AW015313 |
| polymerase I, C | | | | |
| Tissue factor pathway inhibitor | TFPI | 2.435 | 210665—at | AF021834 |
| type 1 tumor necrosis factro receptor shedding aminopeptidase | ARTS-1 | 2.428 | 214012—at | BE551138 |
| regulator | | | | |
| Pro-collagen-lysine, 2-oxoglutarate 5-deoxzgenase 2(lysine | PLOD2 | 2.327 | 202619—s—at | AI754404 |
| hydroxylase) | | | | |
| N-acylsphingosine amidohydrolase (acid ceramidase)-like | ASAHL | 2.270 | 214765—s—at | AK024677 |
| Protein tyrosine phosphatase, receptor type, C | PTPRC | 2.184 | 212588—at | AI809341 |
| Prostaglandin D2 synthase, hematopoietic | PGDS | 2.150 | 206726—at | NM—014485 |
| NAD(P)H dehydrogenase, quinone 1 | DIA4, NMOR1 | 2.116 | 210519—s—at | BC000906 |
| mannosidase alpha class 1A, member 1 | MAN1A1 | 2.048 | 221760—at | BG287153 |
| Myosin VA (heavy polypeptide 12, myoxin) | MYO5A | 2.040 | 204527—at | NM—000259 |
| Extracellular Cell Comp | | | | |
| Macrophage receptor with collagenous structure | MARCO | 13.879 | 205819_at | NM_006770 |
| Asporin (LRR class 1) | PLAP1, FLJ20129 | 2.370 | 219087—at | NM—017680 |
| pro-collagen-lysine, 2-oxoglutarate 5-deoxygenase 2 (lysine | PLOD2 | 2.327 | 202619_s_at | AI754404 |
| hydroxylase) | | | | |
| Collagen, type XI, alpha 1 | STL2, COLL6 | 2.236 | 204320—at | NM—001854 |
| Spondin 2, extracellular matrix protein | DIL1, DIL-1 | 2.056 | 218638—s—at | NM—012445 |
| Intracellular Cell Comp | | | | |
| Nuclear receptor subfamily 1, group D, member 1 | EAR1, hRev | 5.039 | 204760—s—at | NM—021724 |
| NAD(P)H dehydrogenase, quinone 1 | NQO1 | 3.417 | 201468—s—at | NM—000903 |
| Polyadenylate binding protein-interacting protein 1 | PAIP1 | 3.170 | 209064—x—at | AL136920 |
| SAM domain, SH3 domain and nuclear localisation signals, 1 | SAMSN1 | 2.647 | 220330—s—at | NM_022136 |
| NAD(P)H dehydrogenase, quinone 1 | DIA4, NMOR1, | 2.116 | 210519—s—at | BC000906 |
| | NMORI | | | |
| Myosin VA (heavy polypeptide 12, myoxin) | MYO5A | 2.040 | 204527—at | NM—000259 |
| Lymphocyte cytosolic protein 2 | LCP2 | 2.003 | 205269—at | AI123251 |
| Immunity | | | | |
| Interleukin 1 receptor, type II | IL1RB | 4.353 | 211372_s_at | U64094 |
| Allograft inflammatory factor 1 | IBA1, IRT-1 | 4.307 | 209901—x—at | U19713 |
| Proteoglycan 2, bone marrow (natural killer cell activator) | MBP, BMPG | 4.263 | 211743_s_at | BC005929 |
| FYN-binding protein (FYB-120/130) | FYB | 4.158 | 211794_at | AF198052 |
| Major histocompatibility complex, class II, DQ beta 1 | IDDM1, HLA-DQB | 4.038 | 209823—x—at | M17955 |
| HLA class II histocompatibility antigen, DQ (W1.1), beta chain | HLA-DQB1 | 3.955 | 212998—x—at | AI583173 |
| (human) | | | | |
| macrophage scavenger receptor 1 | MSR1 | 3.945 | 214770—at | AI299239 |
| Campath-1 antigen | CDW52 | 3.718 | 34210_at | N90866 |
| Allograft inflammatory factor 1 | AIF1 | 3.147 | 213095—x—at | AF299327 |
| CD69 antigen (p60, early T-cell activation antigen) | CD69 | 2.997 | 209795—at | L07555 |
| [symbol] CXCR4 gene encoding receptor CXCR4. | CXCR4 | 2.831 | 217028—at | AJ224869 |
| T cell receptor beta locus | TRB@ | 2.765 | 211796—s—at | AF043179 |
| CD44 antigen | CD44 | 2.638 | 212063—at | BE903880 |
| FYN-binding protein (FYB-120/130) | FYB | 2.545 | 211795—s—at | AF198052 |
| Interleukin 18 receptor accessory protein | ACPL | 2.489 | 207072_at | NM_003853 |
| Cytokine receptor-like factor 1 | CLF-1 | 2.461 | 206315—at | NM—004750 |
| Toll-like receptor 7 | TLR7 | 2.403 | 220146—at | NM—016562 |
| Coagulation factor III (thromboplastin, tissue factor) | TF, TFA, CD142 | 2.317 | 204363—at | NM—001993 |
| Major histocompatibility complex, class II, DR beta 5 | HLA-DRB5 | 2.284 | 215193—x—at | AJ297586 |
| Complement component 1, q subcomponent, alpha polypeptide | C1QA | 2.244 | 218232—at | NM—015991 |
| Lymphocyte antigen 75 | DEC-205, GP200- | 2.241 | 205668—at | NM—002349 |
| | MR6 | | | |
| Leukotriene b4 receptor (chemokine receptor-like 1) | BLTR, P2Y | 2.222 | 210128_s_at | U41070 |
| Fc fragment of IgG, high affinity Ia, receptor for (CD64) | FCGR1A | 2.111 | 216950—s—at | X14355 |
| T cell receptor delta locus | TRD, TCRD | 2.059 | 217143—s—at | X06557 |
| Integrin, beta 2 (antigen CD18 (p95) | LAD, CD18 | 2.037 | 202803—s—at | NM—000211 |
| Toll-like receptor 2 | TLR2 | 2.033 | 204924—at | NM—003264 |
| Epstein-Barr virus induced gene 2 | EBI2 | 2.004 | 205419—at | NM—004951 |
| Lymphocyte cytosolic protein 2 | LCP2 | 2.003 | 205269—at | AI123251 |
| Membrane | | | | |
| macrophage scavenger receptor 1 | MSR1 | 3.945 | 214770—at | AI299239 |
| Nucleic Acid Binding | | | | |
| Nuclear receptor subfamily 1, group D, member 1 | EAR1, hRe | 5.039 | 204760—s—at | NM—021724 |
| Polyadenylate binding protein-interacting protein 1 | PAIP1 | 3.170 | 209064—x—at | AL136920 |
| RE1-silencing transcription factor | REST | 2.903 | 204535_s_at | AI978576 |
| CCAAT/enhancer binding protein (C/EBP), alpha | CEBP | 2.608 | 204039—at | NM—004364 |
| Zinc finger protein 80 (pT17) | ZNF80 | 2.543 | 207272_at | NM_007136 |
| TATA box binding protein (TBP) - associated factor, RNA | TAF1C | 2.447 | 203937_s_at | AW015313 |
| polymerase I, C | | | | |
| RNA-binding protein gene with multiple splicing | HERMES | 2.401 | 207836—s—at | NM—006867 |
| High-mobility group (nonhistone chromosomal) protein isoforms I | HMGIY | 2.365 | 206074—s—at | NM—002131 |
| and Y | | | | |
| MADS box transcription enhancer factor 2, polypeptide A | RSRFC4, RSRFC9 | 2.238 | 208328—s—at | NM—005587 |
| Transcription factor AP-2 gamma | TFAP2C | 2.233 | 205286—at | U85658 |
| Poly(A)-binding protein, cytoplasmic 3 | PABPC3 | 2.205 | 208113—x—at | NM—030979 |
| Basic helix-loop-helix domain containing, class B, 3 | DEC2, SHARP1 | 2.145 | 221530—s—at | AB044088 |
| MADS box transcription enhancer factor 2, polypeptide A | MEF2A | 2.043 | 214684—at | X63381 |
| eukaryotic translation initiation factor 1A | EIF1A | 2.041 | 201017—at | BE542684 |
| Signal Transduction | | | | |
| Tachykinin, precursor 1 | NK2 | 27.839 | 206552—s—at | NM—003182 |
| LIM protein | LIM | 3.429 | 216804—s—at | AK027217 |
| Placental growth factor, vascular endothelial growth factor-related | PLGF | 3.237 | 209652_s_at | BC001422 |
| protein | | | | |
| CD69 antigen (p60, early T-cell activation antigen) | CD69 | 2.997 | 209795—at | L07555 |
| SAM domain, SH3 domain and nuclear localisation signals, 1 | SAMSN1 | 2.647 | 220330—s—at | NM—022136 |
| G protein-coupled receptor; Human CB1 cannabinoid receptor | CNR1 | 2.620 | 213436—at | U73304 |
| (CNR1) gene | | | | |
| Yamaguchi sarcoma viral related oncogene homolog | LYN | 2.463 | 202625—at | AI356412 |
| MAD (mothers against decapentaplegic, ) homolog 7 | MADH8, SMAD7 | 2.437 | 204790—at | NM—005904 |
| Tissue factor pathway inhibitor | TFPI | 2.435 | 210665—at | AF021834 |
| bone morphogenetic protein | BMP2 | 2.430 | 205289—at | AA583044 |
| V-yes-1 Yamaguchi sarcoma viral related oncogene homolog | JTK8 | 2.357 | 202626—s—at | NM—002350 |
| G-protein coupled receptor 56 | GPR56 | 2.346 | 212070—at | AL554008 |
| Coagulation factor III (thromboplastin, tissue factor) | TF, TFA, CD142 | 2.317 | 204363—at | NM—001993 |
| Prostaglandin E receptor 4 (subtype EP4) | EP4 | 2.291 | 204897—at | NM—000958 |
| ADP ribosylation factor 6 | ARF6 | 2.288 | 214182—at | AA243143 |
| Protein tyrosine phosphatase, receptor type, C | PTPRC | 2.184 | 212588—at | AI809341 |
| Hepatocyte growth factor (hepapoietin A; scatter factor) | HPTA | 2.160 | 210997—at | M77227 |
| docking protein 2 | DOK2 | 2.082 | 214054_at | AI828929 |
| CDC42-binding protein kinase beta (DMPK-like) | MRCKB, KIAA1124 | 2.079 | 217849—s—at | NM—006035 |
| Notch () homolog 3 | NOTCH3 | 2.039 | 203238—s—at | NM—000435 |
| Taste receptor, type 2, member 10 | TRB2, T2R10 | 2.039 | 221397_at | NM_023921 |
| Integrin, beta 2 (antigen CD18 (p95) | LAD, CD18 | 2.037 | 202803—s—at | NM—000211 |
| Milk fat globule-EGF factor 8 protein | MFGE8 | 2.016 | 210605—s—at | BC003610 |
| Epstein-Barr virus induced gene 2 | EBI2 | 2.004 | 205419—at | NM—004951 |
| Lymphocyte cytosolic protein 2 | LCP2 | 2.003 | 205269—at | AI123251 |
| Storage | | | | |
| Milk fat globule-EGF factor 8 protein | MFGE8 | 2.016 | 210605—s—at | BC003610 |
| Structural Protein | | | | |
| Macrophage receptor with collagenous structure | MARCO | 13.879 | 205819_at | NM_006770 |
| Asporin (LRR class 1) | PLAP1, FLJ20129 | 2.370 | 219087—at | NM—017680 |
| Neurofilament 3 (150 kD medium) | NFM, NEFM, NF-M | 2.306 | 205113_at | NM_005382 |
| Collagen, type XI, alpha 1 | STL2, COLL6 | 2.236 | 204320—at | NM—001854 |
| Spondin 2, extracellular matrix protein | DIL1, DIL-1 | 2.056 | 218638—s—at | NM—012445 |
| Transport | | | | |
| sortilin-related receptor, L(DLR class) A repeats-containing | SORL1 | 2.810 | 212560—at | AV728268 |
| chloride intracellular channel 2 | CLIC2 | 2.444 | 213415—at | AI768628 |
| Solute carrier family 16(monocarboxzlic acid tgransporters), | MCT2 | 2.416 | 207057—at | NM—004731 |
| member 7 | | | | |
| Solute carrier family 21 (organic anion transporter), member 9 | OATPB, OATP-B | 2.132 | 203473—at | NM—007256 |

Bold = present in 4 Out of 8 CIDP samples for up-regulated genes

TABLE 4
Down-regulated genes in CIDP compared to NN

| | | Fold | | |
|---|---|---|---|---|
| Gene Description | Common Name | change | Affymetrix | GenBank |
| Cancer | | | | |
| Chromogranin A (parathyroid secretory protein 1) | CGA, CgA | 0.497 | 204697_s_at | NM_001275 |
| Neurofibromin 2 (bilateral acoustic neuroma) | NF2 | 0.211 | 211092_s_at | AF122827 |
| Cell communication | | | | |
| Autocrine motility factor receptor | GP78 | 0.477 | 202203—s—at | NM—001144 |
| Chaperone | | | | |
| DnaJ (Hsp40) homolog, subfamily B, member 4 | HLJ1, DNAJW | 0.387 | 203811_s_at | NM_007034 |
| Enzyme/Metabolism | | | | |
| DKFZP586B1621 protein | DKFZP586B1621 | 0.483 | 218688_at | NM_015533 |
| dicarbonyl/L-xylulose reductase | DCXR | 0.468 | 217973—at | NM—016286 |
| SEE ALSO | PH5P, p193 | 0.440 | 216822_x_at | AL359763 |
| Beta-1,3-glucuronyltransferase 1 | HNK-1, GLCATP, | 0.370 | 219521_at | NM_018644 |
| (glucuronosyltransferase P) | GLCAT-P | | | |
| Calcium/calmodulin-dependent protein kinase (CaM | CAMKB | 0.285 | 211483_x_at | AF081924 |
| kinase) II beta | | | | |
| Cytochrome P450, subfamily IIJ (arachidonic acid | CPJ2 | 0.252 | 205073—at | NM—000775 |
| epoxygenase) polypeptide 2 | | | | |
| Intracellular Cell Comp | | | | |
| Cytokine-like nuclear factor n-pac | N-PAC | 0.496 | 222115—x—at | BC003693 |
| SEE ALSO | PH5P, p193 | 0.440 | 216822_x_at | AL359763 |
| Cytochrome P450, subfamily IIJ (arachidonic acid | CPJ2 | 0.252 | 205073—at | NM—000775 |
| epoxygenase) polypeptide 2 | | | | |
| Nucleic Acid Binding | | | | |
| Sirtuin (silent mating type information regulation 2, | SIR2L3 | 0.489 | 221562_s_at | AF083108 |
| S. cerevisiae, homolog) 3 | | | | |
| Hypothetical protein FLJ22347 | FLJ21850, FLJ22267 | 0.429 | 218965_s_at | NM_022830 |
| Signal Transduction | | | | |
| | SIR2L3 | 0.489 | 221562_s_at | AF083108 |
| Autocrine motility factor receptor | GP78 | 0.477 | 202203—s—at | NM—001144 |
| Mitogen activated protein kinase | MAPK4 | 0.451 | 204707_s_at | BF115223 |
| GABA(A) receptors associated protein like 3 | GABARAPL3 | 0.396 | 211457_at | AF180519 |
| Ganglioside-induced differentiation-associated protein | GDAPILP | 0.389 | 219668_at | NM_024034 |
| 1-like | | | | |
| Calcium/calmodulin-dependent protein kinase (CaM | CAMKB | 0.285 | 211483_x_at | AF081924 |
| kinase) II beta | | | | |
| Purinergic receptor P2Y, G-protein coupled, 2 | P2U, HP2U, P2Y2 | 0.155 | 206277_at | NM_002564 |

Bold = genes present in at least 2 out of 3 NN samples

The most strongly up-regulated genes in CIDP are summarized in Table 5

TABLE 5
up-regulated genes in CIDP compared to normal nerve

| | | | | | Fold | | |
|---|---|---|---|---|---|---|---|
| | OFFICAL NAME | | GeneID # | | increase | SEQ ID NO | |
| | (ALIAS) | DESCRIPTION | (NCBI) | GenBank # | (CIDP) | Polynucleotide | Polypeptide |
| 1 | TAC1 (NK2, NKNA, | Tachykinin, precursor 1 | 6863 | NM_003182 | 27 | 1 | 17 |
| | TAC2) | (substance K, substance P, | | | | | |
| | | neurokinin 1, neurokinin 2, | | | | | |
| | | neuromodulin L, neurokinin | | | | | |
| | | alpha, neuropeptide k, | | | | | |
| | | neuropeptide gamma) | | | | | |
| 2 | NRID1, (EAR1, hRev, | Nuclear receptor subfamily | 9572 | NM_021724 | 5 | 2 | 18 |
| | EAR-1) | 1, group D, member 1 | | | | | |
| 3 | SCD | Stearoyl-CoA desaturase | 6319 | NM_005063 | 16 | 3 | 19 |
| 4 | AIF1 (IRT-1, IBA1) | Allograft inflammatory | 199 | NM_001623 | 4.3 | 4 | 20 |
| | | factor | | | | | |
| 5 | HLA-DQB1 | Major histocompatibility | 3119 | NM_002123 | 4 | 5 | 21 |
| | (IDDM1, HLA-DQB) | complex, class II, DQ beta 1 | | | | | |
| 6 | MSR1 | Macrophage scavenger | 4481 | NM_002445 | 3.9 | 6 | 22 |
| | | receptor 1 | | NM_138715 | | 7 | 23 |
| 7 | XLKD1 | Extracellular link domain | 10894 | NM_006691 | 3 | 8 | 24 |
| | | containing 1 | | | | | |
| 8 | IL1R2 (IL1RB) | Interleukin I receptor, type | 7850 | NM_010555 | 4.3 | 9 | 25 |
| | | II | | | | | |
| 9 | NQO1 | NAD(P)H dehydrogenase, | 1728 | NM_000903 | 3.4 | 10 | 26 |
| | | quinone 1 | | | | | |
| 10 | MARCO | Macrophage receptor with | 8685 | NM_006770 | 14 | 11 | 27 |
| | | collagenous structure | | | | | |
| 11 | ADAMTSL2, (KIAA0605) | ADAMTS-like 2, | 9719 | NM_014694 | 6.5 | 12 | 28 |
| | | KIAA0605 gene product | | | | | |
| 12 | CLCA2 | Chloride channel, calcium | 9635 | NM_006536 | 5.8 | 13 | 29 |
| | | activated, family member 2 | | | | | |
| 13 | PCSK1 (PC1, PC3, NEC1, | Proprotein convertase | 5122 | NM_000439 | 5.6 | 14 | 30 |
| | PC-1) | subtilisin/kexin type 1 | | | | | |
| 14 | PRG2 (MBP, BMPG) | Proteoglygan 2, bone | 5553 | NM_002728 | 4.3 | 15 | 31 |
| | | marrow (naturall killer cell | | | | | |
| | | activator) | | | | | |
| 15 | FYB | FYN-binding protein (FYB- | 2533 | NM_001465 | 4.2 | 16 | 32 |
| | | 120/130 | | | | | |

2) Up-Regulated Genes in VAS Versus NN

In VAS versus NN, 244 genes were differentially regulated. 163 genes were up-regulated and 81 genes were down-regulated. Again, most genes were involved in signal transduction (26%) in immunity (22.9%) and 20% were enzymes. A list of the genes with putative function in the immune stem is given in Table 6.

TABLE 6
Differently regulated genes (DEGs) with putative functions in immunity in vasculitic nerve
(VAS) compared to normal nerve (NN)

| | | Fold | | |
|---|---|---|---|---|
| Gene Description | Common Name | change | Affymetrix | GenBank |
| Heparanase | HPA, HSE1 | 11.941 | 219403_s_at | NM_006665 |
| Allograft inflammatory factor 1 | IBA1, IRT-1 | 10.862 | 209901—x—at | U19713 |
| Campath-1 antigen | CDW52 | 8.957 | 34210—at | N90866 |
| Allograft inflammatory factor 1 | AIF1 | 8.445 | 215051—x—at | BF213829 |
| Allograft inflammatory factor 1 | AIF1 | 8.219 | 213095—x—at | AF299327 |
| major histocompatilbility complex, class II, | HLA-DRB3 | 8.197 | 221491—x—at | AA807056 |
| DR beta 3 | | | | |
| Fc fragment of IgG, high affinity Ia, | FCGR1A | 8.019 | 214511—x—at | L03419 |
| receptor for (CD64) | | | | |
| Complement component 3a receptor 1 | AZ3B, C3AR, HNFAG09 | 7.815 | 209906—at | U62027 |
| lymphocyte antigen 96 | LY96 | 7.512 | 206584—at | NM—015364 |
| Immunoglobulin superfamily, member 6 | DORA | 7.302 | 206420—at | NM—005849 |
| CD69 antigen (p60, early T-cell activation | CD69 | 7.029 | 209795—at | L07555 |
| antigen) | | | | |
| CD163 antigen | M130, MM130 | 6.909 | 215049—x—at | Z22969 |
| Cytokine-like protein C17 | C17 | 6.598 | 219837_s_at | NM_018659 |
| Monokine induced by gamma interferon | CMK, SCYB9 | 6.528 | 203915—at | NM—002416 |
| Fc fragment of IgG, high affinity Ia, | FCGR1A | 6.510 | 216950—s—at | X14355 |
| receptor for (CD64) | | | | |
| Pentaxin-related gene, rapidly induced by | PTX3 | 5.848 | 206157—at | NM—002852 |
| IL-1 beta | | | | |
| Interleukin 7 | IL7 | 5.613 | 206693—at | NM—000880 |
| B-lymphocyte activator macrophage | SBBI42, BLAME | 5.399 | 219386—s—at | NM—020125 |
| expressed | | | | |
| T cell receptor gamma locus | TRG@ | 5.246 | 209813_x_at | M16768 |
| Ectonucleoside triphosphate | CD39, NTPDase-1 | 5.023 | 209474—s—at | U87967 |
| diphosphohydrolase 1 | | | | |
| Cathepsin S | CTSS | 4.921 | 202901—x—at | BC002642 |
| chemokine (C—C motif) receptor 1 | CCR1 | 4.580 | 205098—at | AI421071 |
| Homo sapiens IgH VH gene for | IgH VH | 4.536 | 216510_x_at | AB035175 |
| immunoglobulin heavy chain, partial cds. | | | | |
| chemokine (C—C motif) ligand 3, Small | LD78ALPHA, MIP-1-alpha, CCL3 | 4.099 | 205114—s—at | NM—002983 |
| inducible cytokine A3 (homologous to | | | | |
| mouse Mip-1a) | | | | |
| CD53 antigen | CD53 | 3.741 | 203416—at | NM—000560 |
| Ectonucleoside triphosphate | CD39, NTPDase-1 | 3.706 | 207691—x—at | NM—001776 |
| diphosphohydrolase 1 | | | | |
| Lymphocyte cytosolic protein 2 | LCP2 | 3.589 | 205269—at | AI123251 |
| cytochrome b-245, beta polypeptide | CYBB | 3.570 | 203922—s—at | AI308863 |
| Neutrophil cytosolic factor 2 (65 kD, chronic | NCF2 | 3.555 | 209949—at | BC001606 |
| granulomatous disease, autosomal 2) | | | | |
| Lymphocyte antigen 86 | LY86 | 3.474 | 205859—at | NM—004271 |
| Fc fragment of IgE, high affinity I, receptor | FCER1G | 3.413 | 204232—at | NM—004106 |
| for; gamma polypeptide | | | | |
| arachidonate-5 lipooxygenase | ALOX5 | 3.282 | 214366—s—at | AA995910 |
| Proteoglycan 2, bone marrow (natural killer | MBP, BMPG | 3.101 | 211743_s_at* | BC005929 |
| cell activator, eosinophil granule major basic | | | | |
| protein) | | | | |
| CD86 antigen (CD28 antigen ligand 2, B7-2 | B70, B7-2, LAB72, CD28LG2 | 3.085 | 210895—s—at | L25259 |
| antigen) | | | | |
| Hepcidin antimicrobial peptide | HEPC, LEAP1, LEAP-1 | 3.013 | 220491—at | NM—021175 |
| CD2 antigen (p50), sheep red blood cell | SRBC | 2.978 | 205831—at | NM—001767 |
| receptor | | | | |
| CD84 antigen (leukocyte antigen) | CD84 | 2.972 | 205988—at | NM—003874 |
| sialoadhesin | SN | 2.882 | 44673—at | N53555 |
| Interleukin 8 | IL8 | 2.812 | 202859—x—at | NM—000584 |
| CD163 antigen | M130, MM130 | 2.801 | 216233—at | Z22970 |
| CD86 antigen (CD28 antigen ligand 2, B7-2 | CD86 | 2.767 | 205685—at | BG236280 |
| antigen) | | | | |
| Leukocyte immunoglobulin-like receptor, | ILT1, LIR7, LIR-7 | 2.724 | 211100_x_at | U82278 |
| subfamily A (with TM domain), member 2 | | | | |
| Chemokine (C—C motif) receptor-like 2 | HCR, CKRX, CRAM-A, CRAM-B | 2.652 | 211434—s—at | AF015524 |
| chemokine (C—C motif) ligand 4, Small | CCL4, ACT2, LAG1, Act-2, AT744.1, | 2.639 | 204103—at | NM—002984 |
| inducible cytokine A4 (homologous to | MIP-1-BETA | | | |
| mouse Mip-1b) | | | | |
| CD44 antigen | CD44 | 2.619 | 212063—at | BE903880 |
| Leukocyte immunoglobulin-like receptor, | HM18, ILT3, LIR5, LIR-5 | 2.483 | 210152—at | U82979 |
| subfamily B (with TM and ITIM domains), | | | | |
| member 4 | | | | |
| T cell receptor gamma constant 2 | TCRGC2, TRGC2(2X), TRGC2(3X) | 2.463 | 215806—x—at | M13231 |
| Syndecan 1 | SDC | 2.461 | 201287—s—at | NM—002997 |
| Pre-B-cell colony-enhancing factor | PBEF | 2.365 | 217739—s—at | NM—005746 |
| CD28 antigen (Tp44) | CD28 | 2.268 | 206545—at | NM—006139 |
| Major histocompatibility complex, class I- | MR1 | 2.150 | 207565—s—at | NM—001531 |
| like sequence | | | | |
| IL2-inducible T-cell kinase | EMT, LYK, PSCTK2 | 2.146 | 211339—s—at | D13720 |
| Lymphocyte antigen 75 | DEC-205, GP200-MR6 | 2.063 | 205668_at | NM_002349 |
| Squamous cell carcinoma antigen | SART-2 | 2.031 | 218854—at | NM—013352 |
| recognized by T cell | | | | |
| Interleukin 8 receptor, beta | CXCR2, IL8RA, CMKAR2 | 0.271 | 207008_at | NM_001557 |
| CD24 antigen (small cell lung carcinoma | CD24A | 0.255 | 208651_x_at | M58664 |
| cluster 4 antigen) | | | | |

bold: for up-regulated genes P in 2 out of 3 VAS samples, for down-regulated genes present in 2 out of 3 NAP samples

The 31 most strongly up-regulated genes in vasculitic nerve are summarized in Table 7.

TABLE 7
up-regulated genes in VAS compared to normal nerve

| | OFFICIAL NAMES | | FOLD | GeneID | GenBank | SEQ ID NO | |
|---|---|---|---|---|---|---|---|
| | (ALIASES) | DESCRIPTION | CHANGE | (NCBI) | Number | Polynucleotide | Polypeptide |
| 1 | RGS1 (IER1, BL34, | Regulator of G-protein | 15.5 | 5996 | NM_002922 | 33 | 59 |
| | IR20) | signaling 1 | | | | | |
| 2 | PCSK1 (PC1, PC3) | Proprotein convertase | 14.6 | 5122 | NM_000439 | 14 | 30 |
| | | subtillisin/kexin type 1 | | | | | |
| 3 | HPSE (HPA, HSE1) | Heparanase-1 | 11.9 | 10855 | NM_006665 | 34 | 60 |
| 4 | HTR2B | 5-Hydroxytryptamine | 11.7 | 3357 | NM_000867 | 35 | 61 |
| | | (serotonine) receptor | | | | | |
| | | 2B | | | | | |
| 5 | MSR1 | Macrophage scavenger | 11.0 | 4481 | NM_002445 | 6 | 22 |
| | | receptor 1 | | | NM_138715 | 7 | 23 |
| 6 | AIF1 (AIF-1, IRT-1, | Allograft | 10 | 199 | NM_001623 | 4 | 20 |
| | IBA1) | Inflammatory factor 1 | | | | | |
| 7 | LAMP3 (LAMP, | Lysosomal associated | 10 | 27074 | NM_014398 | 36 | 62 |
| | CDLAMP, TSC40) | membrane protein 3 | | | | | |
| 8 | CLCA2 | Chloride channel | 9.7 | 9635 | NM_006536 | 13 | 29 |
| | | calcium activated | | | | | |
| | | family member 2 | | | | | |
| 9 | CD52 (CDW52, | Campath-1 antigen | 8.9 | 1043 | NM_001803 | 37 | 63 |
| | CD52) | | | | | | |
| 10 | BIRC1 | Baculoviral IAP | 8.5 | 4671 | NM_004536 | 38 | 64 |
| | | repeat-containing 1, | | | | | |
| | | Strong similarity with | | | | | |
| | | neuronal apoptosis | | | | | |
| | | inhibitory protein | | | | | |
| 11 | HLA-DRB3 | Major | 8.2 | 3125 | NM_022555 | 39 | 65 |
| | | histocompatibility | | | | | |
| | | complex, class II, DR | | | | | |
| | | beta 3 | | | | | |
| 12 | F2RL1 | Coagulation factor II | 8.1 | 2150 | NM_005242 | 40 | 66 |
| | | (thrombin) receptor | | | | | |
| | | like | | | | | |
| 13 | FCGRIA, (CD64) | Fc fragment of IgG, | 8 | 2209 | NM_000566 | 41 | 67 |
| | | high affinity Ia, | | | | | |
| | | receptor for (CD64) | | | | | |
| 14 | C3AR1 (AZ3B, | Complement | 7.8 | 719 | NM_004054 | 42 | 68 |
| | C3AR) | component 3a receptor | | | | | |
| 15 | LY96 (MD-2) | Lymphocyte antigen | 7.5 | 23643 | NM_015364 | 43 | 69 |
| | | 96 | | | | | |
| 16 | ADAMDEC1 | Adam-Like, decysin 1 | 7.4 | 27299 | NM_014479 | 44 | 70 |
| 17 | SAMSN1 | SAM domain, SH3 | 7.4 | 64092 | NM_022136 | 45 | 71 |
| | | domain and nuclear | | | | | |
| | | localization signals | | | | | |
| 18 | IGSF6 (DORA,) | Immunoglobulin | 7.3 | 10261 | NM_005849 | 46 | 72 |
| | | superfamily member 6 | | | | | |
| 19 | CD69 | CD69 Antigen | 7 | 969 | NM_001781 | 47 | 73 |
| 20 | CD163 (M130, | CD163 antigen | 6.9 | 9332 | NM_004244 | 48 | 74 |
| | MM130) | | | | | | |
| 21 | KYNU | Kynureninase (L- | 6.6 | 8942 | NM_001032998 | 49 | 75 |
| | | kynurenine hydrolase) | | | | | |
| 22 | CYTL1 (C17) | Cytokine-like 1, | 6.6 | 54360 | NM_018659 | 50 | 76 |
| | | Cytokine-like protein | | | | | |
| | | C17 | | | | | |
| 23 | CXCL9(CMK, | Chemokine (C—X—C | 6.5 | 4283 | NM_002416 | 51 | 77 |
| | SCYB9) | motif) ligand 9, | | | | | |
| | | Monokine induced by | | | | | |
| | | gamma interferon | | | | | |
| 24 | CMAH (CSAH) | Cytidine | 6.2 | 8418 | Ref D86324 | 52 | 78 |
| | | monophosphate-N- | | | | | |
| | | acetylneuraminic acid | | | | | |
| | | hydroxylase | | | | | |
| 25 | GPR65 | G protein-coupled | 6 | 8477 | NM_003608 | 53 | 79 |
| | | receptor 65 | | | | | |
| 26 | PTX3 | Pentaxin-related gene, | 5.8 | 5806 | NM_002852 | 54 | 80 |
| | | rapidly induced by IL- | | | | | |
| | | 1 beta | | | | | |
| 27 | IL7 | Interleukin 7 | 5.6 | 3574 | NM_000880 | 55 | 81 |
| 28 | SLAMF8, (SBBI42, | Slam family member | 5.4 | 56833 | NM_020125 | 56 | 82 |
| | BLAME) | 8, B-lymphocyte | | | | | |
| | | activator macrophage | | | | | |
| | | expressed | | | | | |
| 29 | ENTPD1 (CD39, | Econucleoside | 5 | 953 | NM_001776 | 57 | 83 |
| | NTPDase) | triphosphate | | | | | |
| | | dephosphohydrolase 1 | | | | | |
| 30 | CCR1 | Chemokine receptor 1 | 4.5 | 1230 | NM_001295 | 58 | 84 |

3) Up-Regulated Genes in CIDP and VAS

24 genes were over expressed in both CIDP and VAS compared to NN, most of which appear to be involved in immunity and inflammation. These included the early T-cell activation gene CD69, the allograft inflammatory factor (AIF1) that is up-regulated in vascular damage and CD44, which has a postulated role in matrix adhesion and lymphocyte activation. Four of the most highly expressed genes in CIDP are also among the most highly expressed genes in vasculitic neuropathy. Compare Tables 5 and 7.

4) Up-Regulated Genes in CIDP Versus VAS and NN

3 genes, Stearoyl-CoA desaturase (SCD), NADPH dehydrogenase, quinone 1 (NQO1) and eukaryotic translation initiation factor 1A (EIF1A) were significantly up-regulated in CIDP in comparison to NN or VAS (Welch t-test with log transformed data; p=0.05, fold change 2.0, genes present in at least one sample).

TABLE 8
Genes that are significantly up-regulated in CIDP compared
to both vasculitis and normal nerve

| DESIGNATION | NAME | GeneID # |
|---|---|---|
| SCD | Stearoyl CoA | 6319 |
| | desaturase | |
| NQO1 | NAD(P)H | 1728 |
| | dehydrogenase, | |
| | quinone 1 | |
| NRIDI | Nuclear | 9572 |
| | receptor | |
| | subfamily 1 | |

### Example III

### Expression of Substance P is Increased in CIDP Nerve

As shown in Example II, a study of gene expression profiles of CIDP nerve biopsies in comparison to normal controls, tachykinin precursor I was the most upregulated gene in CIDP, with a 27.8 fold increase in CIDP. One of the polypeptides encoded by the tachykinin precursor 1 gene is substance P. Substance P is an 11 amino acid peptide that is widely present in the peripheral and central nervous systems and is involved in transmission of pain, as well as other functions. To investigate and characterize the expression of substance P in CIDP nerve in comparison to normal nerve, we performed staining of histological samples, using conventional methods.

Formaldehyde-fixed and paraffin-embedded sections of human sural nerve biopsies were deparaffinized and rehydrated by sequential incubation in xylene, ethanol, and PBS. Antigen retrieval was done by incubation in trypsin and endogenous peroxidase was quenched with H₂O₂ in methanol. After blocking non-specific binding with goat serum in PBS, sections were treated with dilutions of either rabbit anti-substance P antibodies or normal rabbit serum. After washing the sections, they were then treated HRP-conjugated goat anti-rabbit IgG in blocking solution. Colorimetric detection of antibody binding was carried out using the 9-ethylcarbazol-3-amine (AEC)/H₂O₂ chromogen/substrate reagent system.

Results: At an antibody dilution of 1:200, strong staining of CIDP nerve was observed, while normal nerve was not appreciably stained. No staining was observed with normal rabbit serum. The pattern of staining indicated increased expression of substance P in internodal regions of CIDP nerves.

### Example IV

### Determination of Increased Likelihood of Having CIDP or Vascsulitic Neuropathy Using Skin Biopsy

Patients who have been diagnosed as having CIDP or vascultic neuropathy are tested essentially as described in Examples I and II, except samples are taken from skin biopsies instead of from sural nerve.

A 3 mm skin sample containing myelinated nerve fibers is obtained using punch biopsy. Total RNA is extracted as previously described for biopsied nerve (Renaud et al, 2005, supra). As biopsied skin has less nerve tissue than biopsied whole nerve, RNAs that are preferentially expressed in nerve are less abundant in skin, and require amplification prior to differential gene expression. As such, expression of markers of interest, including SCD, NQO1, NR1D1, TAC1, MSR1, AIF1, and CLCA1, are quantified by real time RT-PCR, using primers specific for each of the corresponding RNAs, as previously described above for nerve in CIDP or vasculic neuropathy (Renaud et al. 2005, supra), or by Li et al. (2005) Brain 128, 1168-77 for myelin protein RNAs in skin biopsies of patients with Hereditary neuropathies. The results for the genes of interest are normalized against results obtained for endogenous control genes examined in parallel, including S-100, GFAP, actin, and/or GAPDH. In some cases, following amplification, the cRNAs are also quantified by hybridization to probes specific to the genes of interest, arranged in an array.

A statistically significant correlation is observed between expression of the markers and the presence of CIDP or vasculitic neruopathy.

### Example V

### Diagnosis of Generalized Vasculitis or Vasculitic Neuropathy

Patients who have been diagnosed with vasculitis in the absence of neuropathy are tested essentially as described in Example IV, using samples from skin or other affected tissue, such as muscle, lung or kidney.

In vasculitic neuropathy, the vasculitis can also affect blood vessels in tissues other than nerve. The same tissues can also be affected by vasculitis in the absence of neuropathy. The RNAs of interest in tissues affected by vasculitis are sufficiently abundant that differential gene expression analysis does not require pre-amplification of particular genes. The analysis using skin or other affected tissues is therefore the same as described in Examples I and II, in which samples from peripheral nerve were assayed. DNA microarray analysis as well as real time RT-PCR are used to test for increased expression of genes that are up-regulated in vasculitis, including MSR1, AIF1 and CLCA1, among the others described above.

A statistically significant correlation is observed between expression of the markers and the presence of generalized vasculitis.

From the foregoing description, one skilled in the art can easily ascertain the essential characteristics of this invention, and without departing from the spirit and scope thereof, can make changes and modifications of the invention to adapt it to various usage and conditions and to utilize the present invention to its fullest extent. The preceding preferred specific embodiments are to be construed as merely illustrative, and not limiting of the scope of the invention in any way whatsoever. The entire disclosure of all applications (including provisional U.S. patent application Ser. No. 60/657,122, filed Feb. 28, 2005), patents, publications (including reference manuals) and sequences submitted to GenBank, cited above and in the figures, are hereby incorporated in their entirety by reference.

We claim:

1. A composition comprising
at least two isolated nucleic acids of at least about 15 contiguous nucleotides, selected from nucleic acids that correspond to genes 1-15 from Table 5, and/or genes 1-30 from Table 7, and/or the additional genes in Tables 3, 4 and/or 6, and
wherein the total number of isolated nucleic acids in the composition is no more than about 9,000 (e.g. no more than about 5,000; 1,000; 500; 150; 75; 50).

2. The composition of claim 1, wherein the total number of isolated nucleic acids is no more than about 300.

3. The composition of claim 1, which comprises nucleic acids that correspond to
(a) one or more genes selected from NQO1, NR1D1 and SCD, and
(b) one or more genes selected from TAC1 and AIF1.

4. The composition of claim 3, which comprises nucleic acids that correspond to two or more of genes NQO1, NR1D1 and SCD.

5. The composition of claim 3, which comprises nucleic acids that correspond to all three of genes NQO1, NR1D1 and SCD.

6. The composition of claim 3, which further comprises nucleic acids that correspond to one or more of genes MSR1, PCKS1 and CLCA2.

7. The composition of claim 3, which comprises nucleic acids that correspond to SCD, NQO1, NR1D1, TAC1, AIF1, MSR1, PCKS1, CLCA2 and, optionally, one or more control constitutive genes (e.g., actin, GAPDH, S100 and/or GFAP).

8. The composition of claim 3, which further comprises nucleic acids that correspond to one or more additional genes indicated in Table 5.

9. The composition of claim 3, which further comprises nucleic acids that correspond to one or more additional genes indicated in Table 7.

10. The composition of claim 3, which further comprises nucleic acids that correspond to one or more additional genes indicated in Table 3, 4, 5, 6 or 7.

11. The composition of claim 3, which further comprises nucleic acids that correspond to five (or ten) different additional genes from Table 5.

12. The composition of claim 3, which further comprises nucleic acids that correspond to ten (or twenty) different additional genes from Table 7.

13. The composition of claim 1, which comprises nucleic acids of at least about 15 contiguous nucleotides that correspond to:
(a) one, two, three, four or five of genes #1-5 in Table 7; and/or
(b) one, two, three, four or five of genes #6-10 in Table 7; and/or
(c) one, two, three, four or five of genes #11-15 in Table 7; and/or
(d) one, two, three, four or five of genes #16-20 in Table 7; and/or
(e) one, two, three, four or five of genes #21-25 in Table 7; and/or
(f) one, two, three, four or five of genes #25-30 in Table 7,
wherein the composition comprises nucleic acids corresponding to at least 2 (e.g. at least about 3, 5, 10)) different genes, and
wherein if a nucleic acid that corresponds SCD is present, a nucleic acid corresponding to at least one other gene must be present.

14. The composition of claim 13, which comprises at least about ten (or twenty, fifty, one hundred) isolated nucleic acids of at least about 15 contiguous nucleotides corresponding to at least about ten (e.g., up to 30) different genes selected from genes 1-30 of Table 7.

15. The composition of claim 8, wherein the nucleic acids that correspond to genes from Table 5 are selected from
(a) nucleic acids that comprise the sequences of SEQ ID NOs 1-16;
(b) nucleic acids that comprise sequences which are at least about 85% (e.g. 90%, 95%, 98%) identical to the contiguous sequences in (a);
(c) nucleic acids that comprise sequences encoding polypeptides represented by SEQ ID NOs: 17-32;
(d) nucleic acids that comprise sequences of active fragments of the nucleic acids of (a), (b), and/or (c);
(e) nucleic acids that comprise complete complements of the sequences of any of (a), (b), (c), and/or (d); and/or
(f) nucleic acids that comprise sequences of active variants of the nucleic acids of (a), (b), (c), (d), and/or (e);
wherein the nucleic acids can hybridize under conditions of high stringency to nucleic acids represented by SEQ ID NO's 1-16, or to complete complements thereof.

16. The composition of claim 8, wherein the nucleic acids that correspond to genes from Table 5 are active fragments of about 15 to about 50 contiguous nucleotides of nucleic acids that comprise the sequences of SEQ ID NOs 1-16.

17. The composition of claim 9, wherein the nucleic acids that correspond to genes NQO1, NR1D1, SCD or the genes from Table 7 are active fragments of about 15 to about 50 contiguous nucleotides of nucleic acids that comprise the sequences of SEQ ID NOs: 10, 2, 3, or 33-58.

18. The composition of claim 1,
(a) wherein the nucleic acids are in an array (e.g. a microarray), or
(b) wherein the nucleic acids are primers for PCR.

19. A composition comprising
at least two isolated polypeptides selected from polypeptides that correspond to genes 1-15 from Table 5, and/or genes 1-30 from Table 7, and/or the additional genes in Tables 3, 4 and/or 6.

20. A composition comprising
at least two isolated antibodies which are specific for the polypeptides in the composition of claim 19.
